(19) United States
(12) Patent Application Publication
(10) Pub. No.: US 2023/0270842 A1
(43) Pub. Date: Aug. 31, 2023

(54) COMPOSITIONS AND DEVICES FOR VACCINE RELEASE AND USES THEREOF

(71) Applicant: VAXESS TECHNOLOGIES, INC., Cambridge, MA (US)

(72) Inventors: Michael A. Schrader, Watertown, MA (US); Kathryn M. Kosuda, Cambridge, MA (US); Jonathan A. Kluge, Cambridge, MA (US); Kimberly M. Cirelli, Brookline, MA (US); Emily L. Borkowski, Somerville, MA (US); Cassie L. Caudill, Watertown, MA (US); Matthew Dirckx, Medford, MA (US); Nickolas W. Hartman, Houston, TX (US); Livio Valenti, Cambridge, MA (US)

(73) Assignee: VAXESS TECHNOLOGIES, INC., Cambridge, MA (US)

(21) Appl. No.: 17/991,525

(22) Filed: Nov. 21, 2022

Related U.S. Application Data
(63) Continuation of application No. PCT/US2021/033776, filed on May 21, 2021.
(60) Provisional application No. 63/028,390, filed on May 21, 2020.

(51) Int. Cl. A61K 39/215; A61K 39/145; A61P 37/04; A61K 9/00

(52) U.S. Cl. CPC A61K 39/215; A61K 39/145; A61P 37/04; A61K 9/0021

(57) ABSTRACT

Microneedle and microneedle devices including implantable tips (e.g., silk-based tips) for sustained dermal delivery of a coronavirus and/or influenza vaccine, kits, as well as methods of manufacturing and using the same are described herein. In other embodiments, compositions and methods for controlled- or sustained-administration of a coronavirus vaccine and/or an influenza vaccine to provide improved immunogenicity and/or broad-spectrum immunity to a subject are also described.

### CROSS-REFERENCE TO RELATED APPLICATIONS

This application is a continuation application of International Application No. PCT/US2021/033776, filed on May 21, 2021, which claims the benefit of priority to U.S. Provisional Application No. 63/028,390, filed on May 21, 2020, the entire contents of each of which are expressly incorporated herein by reference.

### GOVERNMENT SUPPORT

This invention was made with support from the federal government under Grant Number 1R44AI142948-01A1 awarded by the National Institute of Allergy and Infectious Diseases (NIAID) as a Small Business Innovation Research Grant (SBIR) award, and under Contract Number 75A50120C00160 funded by the Biomedical Advanced Research and Development Authority (BARDA), a component of the Office of the Assistant Secretary for Preparedness and Response in the U.S. Department of Health and Human Services (HHS). The U.S. government has certain rights in the invention.

### FIELD OF THE INVENTION

The present invention generally relates to microneedles (e.g., silk fibroin-based microneedles) configured to release a vaccine, such as a coronavirus vaccine (e.g., a SARS-CoV-2 vaccine, a SARS-CoV vaccine, and/or a MERS-CoV vaccine) and/or an influenza vaccine, and methods of making and using the same.

### BACKGROUND

Sustained delivery of an antigen into the skin to mimic a natural infection can drive a potent immune response, however achieving this delivery is difficult due to the highly effective barrier properties of the skin's outermost layer, the stratum corneum. Microneedles using traditional materials have been investigated in the delivery of therapeutic agents including vaccines, but are commonly associated with various limitations that compromise their production and limit their performance (see, e.g., Donnelly et al. Drug Deliv. 17(4): 187-207, 2010). There remains a strong need for effective vaccine-delivery compositions, devices (e.g., silk-based microneedles), and methods capable of controlling and/or sustaining vaccine release to enhance an immune response in a subject, and improved approaches to the manufacture of such compositions and devices. Additionally, with the growing threat of infections caused by coronaviruses, including the recent coronavirus disease 2019 (COVID-19) pandemic, and the existing threat of influenza, there is a need for effective protection against both coronaviruses and influenza viruses.

### SUMMARY

The present disclosure features microneedles and microneedle devices comprising silk fibroin protein that can be configured to deliver an effective amount of a therapeutic agent, such as a vaccine, to a subject in need thereof.

In one aspect, the present disclosure features microneedles (e.g., silk fibroin-based microneedles), and microneedle devices (e.g., silk fibroin-based microneedle devices), configured to release (e.g., administer), an effective amount of a coronavirus vaccine, antigen, and/or immunogen and/or an influenza vaccine, antigen, and/or immunogen, to a subject (e.g., a human subject). In some embodiments, the microneedles and microneedle devices comprise a coronavirus vaccine, an influenza vaccine, or a combination thereof. The present disclosure is based, at least in part, on the discovery that modulating the kinetics of antigen presentation via, e.g., controlled- and/or sustained release compositions and devices (e.g., microneedles, e.g., silk-based microneedles, and microneedles devices) comprising a vaccine, antigen, and/or immunogen as described herein, can drive a more potent and/or lasting immune response (e.g., a more potent and/or lasting cellular immune response and/or humoral immune response) in a subject, e.g., as compared to the administration of single-dose or bolus administration of the vaccine. In some embodiments, controlled- or sustained-release of a vaccine, antigen, and/or immunogen as described herein can be used to achieve broad spectrum immunity in a subject, and/or protect against strain drift (e.g., by infection mimicry). Without being bound by theory, traditional vaccines administered by injection into non-barrier tissue are typically rapidly cleared from the body, for example, in less than two days. This is often not enough time for immune cells to mount an optimal, broad humoral or cellular immune response, similar to what occurs through sustained, natural exposure to infection. Accordingly, in some embodiments, the microneedles and microneedle devices described herein can be configured to enhance a subject's immune response to a virus via the sustained exposure of lymphoid tissues to a vaccine antigen to more closely mimic the natural exposure to infection.

In another aspect, the present disclosure features microneedles and microneedle devices that enable a single dose, a two dose, or a multi dose administration of a coronavirus vaccine and/or an influenza vaccine, to provide protection against infection, e.g., by a coronavirus and/or an influenza virus, in the subject. The coronavirus vaccine and/or influenza vaccine may be administered one or more times as a patch, e.g., a single patch, to achieve immunity. The patch may be administered by a health-care professional or may be self-administered. In some embodiments, the microneedles and microneedle devices described herein are shelf stable, e.g., the coronavirus vaccine and/or influenza vaccine within the microneedle or microneedle device may retain activity (e.g., immunogenicity) following a period of time in storage at various environmental conditions, e.g., for a period of at least 2 weeks at room temperature (about 25° C.). In some embodiments, the microneedles and microneedle devices described herein stabilize and maintain the activity of an encapsulated mRNA, e.g., for a period of at least 2 weeks at room temperature (about 25° C.).

In certain embodiments, the microneedles and microneedle devices described herein may be configured to deliver adjuvanted or unadjuvanted vaccine. In certain embodiments, the microneedles and microneedle devices described herein may be configured to deliver adjuvanted or unadjuvanted coronavirus vaccine, antigen, and/or immunogen. In certain embodiments, the microneedles and microneedle devices described herein may be configured to deliver adjuvanted or unadjuvanted influenza vaccine, antigen, and/or immunogen.

In a particular embodiment, the microneedles and microneedle devices contain a vaccine that is free of adjuvant. In a particular embodiment, the microneedles and microneedle devices contain a coronavirus vaccine, antigen, and/or immunogen that is free of adjuvant. In a particular embodiment, the microneedles and microneedle devices contain an influenza vaccine, antigen, and/or immunogen that is free of adjuvant.

In particular, the microneedles and microneedle devices described herein may be configured to deliver unadjuvanted recombinant protein-based vaccines to achieve an immune response that is substantially similar to or greater than the immune response which is achieved using traditional single-dose or bolus administration of the adjuvanted vaccine. In particular, the microneedles and microneedle devices described herein may be configured to deliver unadjuvanted recombinant protein-based coronavirus vaccines to achieve an immune response that is substantially similar to or greater than the immune response which is achieved using traditional single-dose or bolus administration of the adjuvanted coronavirus vaccine. In particular, the microneedles and microneedle devices described herein may be configured to deliver unadjuvanted recombinant protein-based influenza vaccines to achieve an immune response that is substantially similar to or greater than the immune response which is achieved using traditional single-dose or bolus administration of the adjuvanted influenza vaccine.

Without being bound by theory, unadjuvanted recombinant protein-based vaccines delivered using traditional single-dose or bolus administration are typically less effective at achieving an optimal, broad humoral or cellular immune response as compared to use of the adjuvanted vaccine. For example, many recombinant molecules or subunits of pathogens currently under investigation as vaccine antigens have shown little or no inherent immunostimulatory properties, and are therefore typically administered in combination with a potent immunologic adjuvant that can increase and direct vaccine-specific immunity.

Accordingly, in some embodiments, the microneedles and microneedle devices described herein obviate the need to use an adjuvant in combination with a recombinant protein-based vaccine to achieve an immune response.

In certain embodiments, the microneedles and microneedle devices described herein may comprise an mRNA, such as an mRNA vaccine. In particular embodiments, the microneedles and microneedle devices described herein can comprise an mRNA encoding a coronavirus antigen or immunogen and/or an influenza antigen or immunogen. Without being bound by theory, mRNA therapeutics are typically associated with instability and inefficient in vivo delivery, which limits their availability for use in treating human disease. In particular, mRNA vaccine formulations typically require ultra-cold storage at temperatures of −80° C. to ensure stability and prevent degradation during storage, transport, and administration, which can be challenging to maintain on a large-scale basis, particularly in times of a pandemic.

Accordingly, in some embodiments, the microneedles and microneedle devices described herein can improve the storage stability of mRNA therapeutics, including mRNA vaccines, by preventing and/or reducing degradation of an mRNA during prolonged storage. In particular embodiments, the microneedles and microneedle devices described herein can improve the storage stability of mRNA therapeutics, including mRNA vaccines, by preventing and/or reducing degradation of an mRNA during prolonged storage at a temperature of about 4° C., about 25° C., about 37° C., and/or about 45° C. In some embodiments, the microneedles and microneedle devices described herein can improve the storage stability of inherently unstable mRNA therapeutics, including mRNA vaccines, by preventing and/or reducing degradation of an mRNA by at least about 50% (e.g., about 50%, 60%, 70%, 80%, 90%, 92%, 94%, 96%, 98%, 99%, 99.5%, or more) during prolonged storage at a temperature of about 4° C., about 25° C., about 37° C., and/or about 45° C., e.g., as compared to the mRNA therapeutic stored in the absence of a microneedle or microneedle device described herein. In some embodiments, the microneedles and microneedle devices described herein can improve the storage stability of inherently unstable mRNA therapeutics, including mRNA vaccines, such that the mRNA retains at least about 50% (e.g., about 50%, 60%, 70%, 80%, 90%, 92%, 94%, 96%, 98%, 99%, 99.5%, or more) of its original bioactivity (e.g., ability to express an encoded amino acid sequence) after storage for a period of 2 or more weeks, e.g., at a temperature of about 4° C., about 25° C., about 37° C., and/or about 45° C. In some embodiments, the microneedles and microneedle devices described herein can improve the storage stability of mRNA coronavirus vaccines, e.g., by preventing and/or reducing degradation of an the mRNA coronavirus vaccine during prolonged storage at a temperature of about 4° C., about 25° C., about 37° C., and/or about 45° C. In some embodiments, the microneedles and microneedle devices described herein can improve the storage stability of mRNA influenza vaccines, e.g., by preventing and/or reducing degradation of an mRNA influenza vaccine during prolonged storage at a temperature of about 4° C., about 25° C., about 37° C., and/or about 45° C.

Those skilled in the art will recognize, or be able to ascertain using no more than routine experimentation, many equivalents to the specific embodiments of the invention described herein. Such equivalents are intended to be encompassed by the following embodiments (E).
E1. A microneedle device (e.g., a microneedle patch) comprising a plurality of microneedles, wherein the plurality of microneedles comprises:
   a first microneedle comprising a coronavirus antigen (e.g., one or more coronavirus antigens, e.g., a SARS-CoV-2 antigen), or a vaccine preparation thereof (“a coronavirus vaccine”); and
   optionally, a second microneedle comprising an influenza antigen (e.g., one or more influenza antigens, or a vaccine preparation thereof (“an influenza vaccine”));
   optionally, wherein the microneedle device is configured to deliver to a subject the coronavirus antigen or the coronavirus vaccine and, optionally, the influenza antigen or the influenza vaccine, e.g., in an amount sufficient to induce an immune response (e.g., a humoral and/or cellular immune response).
E2. The microneedle device of embodiment E1, wherein the first and/or second microneedle in the plurality of microneedles comprises:
   (i) a base (e.g., a dissolvable base);
   (ii) a tip (e.g., an implantable tip) applied to the base; and
   (iii) (optional) a backing applied to the base.
E3. A plurality of microneedles, comprising:
   a first microneedle comprising a coronavirus antigen (e.g., one or more coronavirus antigens, e.g., a SARS-CoV-2 antigen), or a vaccine preparation thereof (“a coronavirus vaccine”); and
   optionally, a second microneedle comprising an influenza antigen (e.g., one or more influenza antigens, or a vaccine preparation thereof (“an influenza vaccine”));
   optionally, wherein the first and second microneedles are configured to deliver to a subject the coronavirus antigen or the coronavirus vaccine and, optionally, the influenza antigen or the influenza vaccine, in an amount sufficient to sufficient to induce an immune response (e.g., a humoral and/or cellular immune response).
E4. The microneedle device, or the plurality of microneedles, of any one of the preceding embodiments, wherein the first and/or second microneedle comprises a silk fibroin, e.g., a regenerated silk fibroin and/or recombinant silk fibroin.
E5. The microneedle device, or the plurality of microneedles, of any one of the preceding embodiments, wherein the microneedle tip is a silk fibroin tip (e.g., an implantable silk fibroin tip).
E6. A microneedle (e.g., a first microneedle) comprising:
   (i) a base (e.g., a dissolvable base);
   (ii) a silk fibroin tip (e.g., an implantable silk fibroin tip) comprising silk fibroin applied to the base; and
   (iii) (optional) a backing applied to the base;
   wherein the silk fibroin tip comprises a silk fibroin, e.g., a regenerated silk fibroin and/or a recombinant silk fibroin; and
   wherein the microneedle comprises a coronavirus antigen, e.g., one or more coronavirus antigens (e.g., one or more of: a SARS-CoV-2 antigen, a SARS-CoV antigen, or a MERS-CoV antigen), or a vaccine preparation thereof (“a coronavirus vaccine”), and
   optionally the microneedle is configured to deliver to a subject the coronavirus antigen or the coronavirus vaccine, e.g., in an amount sufficient to induce an immune response (e.g., a humoral and/or cellular immune response).
E7. A microneedle device, or a plurality of microneedles, comprising the microneedle of embodiment E6, and optionally further comprising a second microneedle or plurality of microneedles comprising an influenza antigen (e.g., one or more influenza antigens, or a vaccine preparation thereof (“an influenza vaccine”)).
E8. A microneedle device (e.g., a microneedle patch) comprising a plurality of silk fibroin-based microneedles, wherein:
   the plurality of microneedles (e.g., the plurality of first microneedles) comprises a coronavirus antigen, e.g., one or more coronavirus antigens (e.g., one or more of: a SARS-CoV-2 antigen, a SARS-CoV antigen, or a MERS-CoV antigen), or a vaccine preparation thereof (“a coronavirus vaccine”); and
   optionally wherein the microneedle device is configured to deliver to a subject the coronavirus antigen or the coronavirus vaccine, e.g., in an amount sufficient to induce an immune response (e.g., a humoral and/or cellular immune response).
E9. The microneedle device of embodiment E8, wherein the microneedle comprises:
   (i) a base (e.g., a dissolvable base),
   (ii) a silk fibroin tip (e.g., an implantable silk fibroin tip) comprising a silk fibroin applied to the base; and
   (iii) optionally a backing applied to the base.
E10. The microneedle device of embodiment E8 or E9, wherein the microneedle device further comprises a second microneedle or plurality of microneedles comprising an influenza antigen, e.g., an influenza vaccine.
E11. The microneedle device, plurality of microneedles, or microneedle of any one of the preceding embodiments, wherein the microneedle tip comprises the coronavirus vaccine or the influenza vaccine.
E12. The microneedle device, or plurality of microneedles, of any one of embodiment E1-E5, E7, or E10, wherein the microneedle tip comprises the coronavirus vaccine and the influenza vaccine.
E13. The microneedle device, or plurality of microneedles, of any one of embodiment E1-E5, E7 or E10, wherein the microneedle base (e.g., dissolvable base) comprises the coronavirus vaccine and/or the influenza vaccine.
E14. The microneedle device, or plurality of microneedles, of any one of embodiments E1-E5, or E7-E13, wherein the first and/or second microneedle contains one antigen per microneedle, e.g., one vaccine per microneedle.
E15. The microneedle device, or plurality of microneedles, of any one of embodiments E1-E5 or E7-E13, wherein the first microneedle comprises a combination of antigens derived from the same coronavirus (e.g., a combination of SARS-CoV-2 antigens), or from different coronaviruses, e.g., different betacoronavirus.
E16. The microneedle device, or plurality of microneedles, of any one of embodiments E1-E5, E7, E10-E13, or E15, wherein the second microneedle comprises a combination of antigens derived from the same influenza virus, or from different influenza viruses.
E17. The microneedle device, or plurality of microneedles, of any one of embodiment E1-E5, E7, or E10-E16, wherein the device or plurality further comprises a plurality of additional microneedles (“additional plurality”), wherein one or more microneedles (e.g., each microneedle) in the plurality of additional microneedles comprises an influenza vaccine, e.g., one, two, three, four or more influenza vaccines.
E18. The microneedle device, or plurality of microneedles, of embodiment E17, wherein the additional plurality comprises one or more microneedles comprising the influenza vaccine in combination, e.g., co-formulated, with the coronavirus vaccine, in the same microneedle.
E19. The microneedle device, or plurality of microneedles, of embodiment E18, wherein each microneedle of the additional plurality comprises the coronavirus vaccine and the influenza vaccine, e.g., one, two, three, four or more influenza vaccines, in the same microneedle.
E20. The microneedle device, or plurality of microneedles, of embodiment E17, wherein the plurality of (e.g., each of) the additional microneedles comprises two, three, four or more of the additional influenza vaccines present in combination, e.g., co-formulated, in the same microneedle, e.g., separately from the coronavirus vaccine.
E21. The microneedle device, or plurality of microneedles, of embodiment E17, wherein each of the one or more influenza vaccines and the coronavirus vaccine are separately formulated into individual microneedles.
E22. The microneedle device, or plurality of microneedles, of any one of embodiments E1-E5 or E7-E21, wherein the plurality of the microneedles comprises at least two, three, four, five, six or more antigens (e.g., at least 5 antigens).
E23. The microneedle device, or plurality of microneedles, of embodiment E22, wherein a portion or all the microneedles in the plurality are the same.
E24. The microneedle device, or plurality of microneedles, of any one of embodiments E1-E5, E7, or E10-E23, wherein the plurality of the microneedles comprises the coronavirus antigen and the influenza antigen, e.g., at least one, two, three, four or five or more influenza antigens, e.g., at least 4 influenza antigens.
E25. The microneedle device, or plurality of microneedles, of embodiment E24, wherein a portion or each of the microneedles in the plurality comprises a combination of the coronavirus antigen and the influenza antigen, e.g., at least one, two, three, four or five or more influenza antigens, e.g., at least 4 influenza antigens.
E26. The microneedle device, or plurality of microneedles, of embodiment E25, wherein each of microneedles in the plurality comprises the combination, e.g., the coronavirus and the influenza antigens are co-formulated, e.g., all microneedles are the same.
E27. The microneedle device, or plurality of microneedles, of embodiment E24, wherein at least two, three, four or five or more of the antigens, e.g., each of the antigens, is individually formulated, e.g., one antigen per microneedle.
E28. The microneedle device, or plurality of microneedles, of embodiment E27, which comprises at least five different microneedles, each microneedle comprising at least one different antigen.
E29. The microneedle device, or plurality of microneedles, of any one of embodiments E1-E5, E7, E10-E28, wherein the plurality comprises at least one coronavirus antigen and at least two, three, four or five or more influenza antigens, e.g., at least 4 influenza antigens.
E30. The microneedle device, or plurality of microneedles, of embodiment E29, wherein each of the antigens is individually formulated.
E31. The microneedle device, or plurality of microneedles, of any one of embodiments E1-E5 or E7-E30, wherein the plurality of microneedles comprises at least 10%, 20%, 30%, 40%, 50% 60%, 70%, 80%, 90% or more (e.g., at least 20%) microneedles which comprise the coronavirus antigen, e.g., one or more coronavirus antigens.
E32. The microneedle device, or plurality of microneedles, of any one of embodiments E1-E5, E7, or E10-30, wherein the plurality of microneedles comprises at least 10%, 20%, 30%, 40%, 50% 60%, 70%, 80%, 90% or more (e.g., at least 80%) microneedles which comprise the influenza antigen, e.g., one, two, three or four influenza antigens.
E33. The microneedle device, or plurality of microneedles, of any one of embodiments E1-E5 or E7-32, wherein the plurality of microneedles comprises:
   (i) at least 10% microneedles each comprising the coronavirus antigen, e.g. one or more coronavirus antigens; and at least at least 90% of the microneedles comprising the influenza antigen, e.g., one, two, three or four influenza antigens;
   (ii) at least 20% microneedles each comprising the coronavirus antigen, e.g. one or more coronavirus antigens; and at least at least 80% of the microneedles comprising the influenza antigen, e.g., one, two, three or four influenza antigens; or
   (iii) at least 30% microneedles each comprising the coronavirus antigen, e.g. one or more coronavirus antigens; and at least at least 70% of the microneedles comprising the influenza antigen, e.g., one, two, three or four influenza antigens.
E34. The microneedle device, the plurality of microneedles, or the microneedle of any one of the embodiments, wherein the coronavirus vaccine comprises a SARS-CoV-2 antigen (e.g., SARS-CoV-2-S1 or a subunit thereof), MERS-CoV antigen (e.g., MERS-CoV-S1 or a subunit thereof), a SARS-CoV antigen (e.g., SARS-CoV-S1 or a subunit thereof), or a combination thereof.
E35. The microneedle device, the plurality of microneedles, or the microneedle of any one of the preceding embodiments, wherein the coronavirus vaccine comprises a SARS-CoV-2 vaccine, e.g., a SARS-CoV-2 gene product (e.g., a SARS-CoV-2 protein, or a nucleic acid (e.g., mRNA, DNA) encoding a SARS-CoV-2 protein), a virus or a viral particle (e.g., inactivated or attenuated SARS-CoV-2 virus), or a viral vector, or a combination thereof.
E36. The microneedle device, the plurality of microneedles, or the microneedle of any one of the preceding embodiments, wherein the SARS-CoV-2 vaccine is chosen from a SARS-CoV-2 spike protein or a subunit thereof, a SARS-CoV-2 nucleocapsid protein, an inactivated virus (e.g., inactivated SARS-CoV-2, e.g., UV-inactivated SARS-CoV-2), a viral vector (e.g., a non-replicating viral vector or a replicating viral vector), a virus-like particle, DNA (e.g., DNA plasmids), RNA (e.g., messenger RNA (mRNA), self-amplifying mRNA (saRNA), nucleoside-modified mRNA (modRNA), or uridine-containing mRNA (uRNA)), and/or an adenovirus vector (e.g., adenovirus type 5 vector, e.g., Ad5-nCoV), or a combination thereof.
E37. The microneedle device, the plurality of microneedles, or the microneedle of any one of the preceding embodiments, wherein the coronavirus vaccine comprises a live virus (e.g., a live modified virus, e.g., a live modified orthopoxvirus such as horsepox virus, comprising a coronavirus antigen).
E38. The microneedle device, the plurality of microneedles, or the microneedle of any one of embodiments E1-E36, wherein the coronavirus vaccine does not comprise a live virus.
E39. The microneedle device, the plurality of microneedles, or the microneedle of any one of embodiments E1-E36, wherein the coronavirus vaccine is selected from the group consisting of a coronavirus spike protein (e.g., a pre-fusion SARS-CoV-2 spike protein), an inactivated SARS-CoV-2 (e.g., UV inactivated SARS-CoV-2, or PiCoVacc); mRNA (e.g., mRNA encoding a coronavirus protein, e.g., mRNA encoding the SARS-CoV-2 spike protein, or a subunit thereof, e.g., mRNA-1273); an adenovirus vector (e.g., an adenovirus type 5 vector that expresses a coronavirus protein (e.g., the SARS-CoV-2 spike protein or a subunit thereof), e.g., Ad5-nCoV); a DNA plasmid (e.g., a DNA plasmid encoding the SARS-CoV-2 spike protein or a subunit thereof), e.g., INO-4800); a dendritic cell (e.g., a dendritic cell modified with a lentiviral vector to express a SARS-CoV-2 minigene), e.g., LV-SMENP-DC); and/or an artificial antigen-presenting cell (aAPC), e.g., an aAPC modified with a lentiviral vector to express a SARS-CoV-2 minigene), or a combination thereof.
E40. The microneedle device, the plurality of microneedles, or the microneedle of any one of the preceding embodiments, wherein the coronavirus vaccine comprises a spike protein, e.g., a pre-fusion SARS-CoV-2 spike protein; or a subunit thereof.
E41. The microneedle device, the plurality of microneedles, or the microneedle of any one of the preceding embodiments, wherein the coronavirus vaccine comprises:
   (i) a whole spike protein, a stabilized spike protein, a locked spike protein, a spike protein subunit, or a receptor-binding domain (RBD) from a spike protein; or
   (ii) a vector (e.g., adenovirus type-5 vector), RNA (e.g., mRNA, saRNA, modRNA, or uRNA) or DNA (e.g., DNA plasmids) that encodes a spike protein or subunit thereof, e.g., a whole spike protein, a stabilized spike protein, a locked spike protein, a spike protein subunit, or a receptor-binding domain (RBD) from a spike protein.
E42. The microneedle device, the plurality of microneedles, or the microneedle of any one of the preceding embodiments, wherein the coronavirus vaccine comprises a pre-fusion SARS-CoV-2 spike protein.
E43. The microneedle device, the plurality of microneedles, or the microneedle of any one of the preceding embodiments, wherein the coronavirus vaccine comprises an inactivated SARS-CoV-2, e.g., a UV inactivated SARS-CoV-2.
E44. The microneedle device, the plurality of microneedles, or the microneedle of any one of the preceding embodiments, wherein the coronavirus vaccine comprises a UV inactivated SARS-CoV-2.
E45. The microneedle device, the plurality of microneedles, or the microneedle of any one of the preceding embodiments, wherein the coronavirus vaccine comprises a recombinant protein, e.g. a recombinant SARS-CoV-2 spike protein, or a subunit thereof.
E46. The microneedle device, the plurality of microneedles, or the microneedle of any one of the preceding embodiments, wherein the coronavirus vaccine comprises a coronavirus-derived protein, e.g., a pre-fusion SARS-CoV-2 spike protein, that comprises a trimeric structure.
E47. The microneedle device, the plurality of microneedles, or the microneedle of any one of the preceding embodiments, wherein the coronavirus vaccine comprises a lipid nanoparticle (LNP) formulation, e.g., a coronavirus vaccine encapsulated by an LNP, e.g., an mRNA vaccine encapsulated by an LNP.
E48. The microneedle device, the plurality of microneedles, or the microneedle of any one of the preceding embodiments, wherein the coronavirus vaccine comprises mRNA-1273, BNT162, INO-4800, Ad26 SARS-CoV-2, TNX-1800, or PiCoVacc, or a combination thereof.
E49. The microneedle device, the plurality of microneedles, or the microneedle of any one of the preceding embodiments, wherein the coronavirus vaccine comprises SARS-CoV-2-S1, SARS-CoV-2-S1fRSO9, or a combination thereof.
E50. The microneedle device, the plurality of microneedles, or the microneedle of any one of the preceding embodiments, wherein the coronavirus vaccine comprises MERS-S1, MERS-S1f, MERS-S1fRSO9, or MERS-S1ffliC, or a combination thereof.
E51. The microneedle device, or plurality of microneedles, of any one of embodiments E1-E5, E7, or E10-E50, wherein the influenza vaccine comprises a univalent (e.g., monovalent) or multivalent influenza vaccine (e.g., a bivalent, trivalent, quadrivalent (or tetravalent), or pentavalent influenza vaccine).
E52. The microneedle device, or the plurality of microneedles, of any one of embodiments E1-E5, E7, or E10-E51, which is configured to deliver (e.g., release) two or more influenza antigens (e.g., three or more, or four or more antigens), e.g., to protect against two or more different influenza viruses.
E53. The microneedle device, or the plurality of microneedles, of any one of embodiments E1-E5, E7, or E10-E52, wherein said plurality of microneedles comprises a third microneedle comprising an influenza antigen that is different from the influenza antigen in the second microneedle.
E54. The microneedle device, or the plurality of microneedles, of embodiment E53, wherein said plurality of microneedles comprises a fourth microneedle comprising an influenza antigen that is different from the influenza antigen in the second and third microneedles.
E55. The microneedle device, or the plurality of microneedles, of embodiment E53 or E54, wherein said plurality of microneedles comprises a fifth microneedle comprising an influenza antigen that is different from the influenza antigen present in the second, third, and fourth microneedles.
E56. The microneedle device, or the plurality of microneedles, of any one of embodiments E53-E55, wherein:
   (i) the combination of influenza antigens in the second and third microneedle comprises a bivalent influenza vaccine;
   (ii) the combination of influenza antigens in the second, third, and fourth microneedle comprises a trivalent influenza vaccine; and/or
   (iii) the combination of influenza antigens in the second, third, fourth, and fifth microneedles comprises a quadrivalent influenza vaccine.
E57. The microneedle device, or the plurality of microneedles, of any one of embodiments E1-E5, E7, or E10-E56, wherein the influenza vaccine comprises an influenza A vaccine, an influenza B vaccine, an influenza C vaccine, and/or an influenza D vaccine.
E58. The microneedle device, or the plurality of microneedles, of any one of embodiments E1-E5, E7, or E10-E57, wherein the influenza vaccine comprises an influenza A vaccine, optionally wherein the influenza A vaccine:
   (i) is an H1N1 (e.g., A/Michigan and/or A/California) vaccine; and/or
   (ii) an H3N2 vaccine (e.g., A/Hong Kong and/or A/Switzerland).
E59. The microneedle device, or the plurality of microneedles, of any one of embodiments E1-E5, E7, or E10-E58, wherein the influenza vaccine comprises an influenza B vaccine, optionally wherein the influenza B vaccine:
   (i) is a B/Yamagata lineage (e.g., B/Phuket); and/or
   (ii) is a B/Victoria lineage (e.g., B/Brisbane) vaccine.
E60. The microneedle device, or the plurality of microneedles, of any one of embodiments E1-E5, E7, or E10-E59, wherein the influenza vaccine comprises an influenza A vaccine (e.g., a H1N1 vaccine and/or a H3N2 vaccine) and an influenza B vaccine (e.g., a B/Yamagata lineage and/or a B/Victoria lineage vaccine).
E61. The microneedle device, or the plurality of microneedles, of any one of embodiments E1-E5, E7, or E10-E60, wherein the plurality of microneedles comprises a 4:1 ratio of influenza vaccines to coronavirus vaccine.
E62. The microneedle device, or the plurality of microneedles, of any one of embodiments E1-E5, E7, or E10-E61, wherein the combination of the coronavirus vaccine and influenza vaccines comprises a pentavalent vaccine.
E63. The microneedle device, the plurality of microneedles, or the microneedle of any one of the preceding embodiments, configured to result in a single dose protection, e.g., immunity, to a coronavirus and/or influenza virus.
E64. The microneedle device, or plurality of microneedles, of any one of embodiments E1-E5, E7, or E10-63, wherein the microneedle device is configured to deliver to the subject the influenza vaccine in an amount sufficient to induce an immune response, e.g., a humoral and/or cellular immune response.
E65. The microneedle device, the plurality of microneedles, or the microneedle of any one of the preceding embodiments, wherein the microneedle is configured to pierce a biological barrier (e.g., skin).
E66. The microneedle device, the plurality of microneedles, or the microneedle of any one of the preceding embodiments, wherein microneedle is configured to implant the microneedle tip (e.g., silk fibroin tip) into a biological barrier (e.g., skin) of a subject.
E67. The microneedle device, the plurality of microneedles, or the microneedle of any one of the preceding embodiments, wherein the microneedle is configured to achieve a local and/or a systemic delivery (e.g., release) of the coronavirus vaccine and/or the influenza vaccine to the subject.
E68. The microneedle device, the plurality of microneedles, or the microneedle of any one of the preceding embodiments, wherein the microneedle is configured to deliver an effective amount of the coronavirus vaccine and/or the influenza vaccine to the subject.
E69. The microneedle device, the plurality of microneedles, or the microneedle of any one of the preceding embodiments, wherein the silk fibroin comprises a regenerated silk fibroin and/or a recombinant silk fibroin.
E70. The microneedle device, the plurality of microneedles, or the microneedle of any one of the preceding embodiments, wherein the device is configured for sustained release of the coronavirus vaccine and/or the influenza vaccine.
E71. The microneedle device, the plurality of microneedles, or the microneedle of embodiment E70, wherein the sustained release comprises a substantially continuous low dose administration of the coronavirus vaccine and/or the influenza vaccine, e.g., between about 1 ug to about 500 ug of the coronavirus vaccine and/or the influenza vaccine is released over a period of time comprising at least about 4 days (e.g., about 4, 5, 6, 7, 8, 9, 10, 11, 12, 13, 14, 15 or more days, e.g., between about 5 days and about 25 days, between about 10 days and about 20 days, between about 10 days and about 15 days, between about 12 days and about 16 days, or between about 14 days and about 15 days).
E72. The microneedle device, the plurality of microneedles, or the microneedle of embodiment E70 or E71, wherein the sustained release comprises a continuous administration of about a greater than 0% portion to about a 100% portion of a total amount of coronavirus vaccine and/or the influenza vaccine present in the microneedle tip.
E73. The microneedle device, the plurality of microneedles, or the microneedle of any one of the preceding embodiments, wherein the microneedle is configured to release the coronavirus vaccine and/or the influenza vaccine into the skin of a subject over a period of time comprising at least about 4 days (e.g., about 4, 5, 6, 7, 8, 9, 10, 11, 12, 13, 14, 15 or more days, e.g., between about 5 days and about 25 days, between about 10 days and about 20 days, between about 10 days and about 15 days, between about 12 days and about 16 days, or between about 14 days and about 15 days).
E74. The microneedle device, the plurality of microneedles, or the microneedle of any one of the preceding embodiments, wherein the microneedle is configured to release the coronavirus vaccine and/or the influenza vaccine into the skin of a subject over a period of time comprising about 1 week to about 2 weeks (e.g., about 7, 8, 9, 10, 11, 12, 13, or 14 days).
E75. The microneedle device, the plurality of microneedles, or the microneedle of any one of embodiments E1-E5, E7, or E10-E74, wherein the release of the coronavirus vaccine occurs at substantially the same rate (e.g., concurrently) with the release of the influenza vaccine.
E76. The microneedle device, the plurality of microneedles, or the microneedle of any one of embodiments E1-E5, E7, or E10-E74, wherein the release of the coronavirus vaccine occurs at a different rate than the release of the influenza vaccine, such that the coronavirus vaccine is released substantially before or substantially after the release of the influenza vaccine.
E77. The microneedle device, the plurality of microneedles, or the microneedle of any one of the preceding embodiments, wherein the coronavirus vaccine and/or the influenza vaccine is stabilized by the microneedle, e.g., retains at least 70% of its original bioactivity (e.g., immunogenicity) after storage for a period of 2 or more weeks, e.g., at room temperature (e.g., about 25° C.).
E78. The microneedle device, the plurality of microneedles, or the microneedle of any one of the preceding embodiments, wherein the coronavirus vaccine and/or the influenza vaccine retains at least 70%, 80%, or 90% of its original bioactivity (e.g., immunogenicity) after storage at about 25° C. for 2 weeks; at least 70%, 80%, or 90% of its original bioactivity (e.g., immunogenicity) after storage at about 25° C. for 4 weeks; at least 70%, 80%, or 90% of its original bioactivity (e.g., immunogenicity) after storage at about 25° C. for 8 weeks; and/or at least 70%, 80%, or 90% of its original bioactivity (e.g., immunogenicity) after storage at about 25° C. for 12 weeks.
E79. The microneedle device, the plurality of microneedles, or the microneedle of any one of the preceding embodiments, wherein the coronavirus vaccine and/or the influenza vaccine retains at least 60%, 70%, or 80% of its original bioactivity (e.g., immunogenicity) after storage at about 37° C. for 2 weeks; at least 50%, 60%, or 70% of its original bioactivity (e.g., immunogenicity) after storage at about 37° C. for 4 weeks; at least 50%, 60%, or 70% of its original bioactivity (e.g., immunogenicity) after storage at about 37° C. for 8 weeks; and/or at least 30%, 40%, or 50% of its original bioactivity (e.g., immunogenicity) after storage at about 37° C. for 12 weeks.
E80. The microneedle device, the plurality of microneedles, or the microneedle of any one of the preceding embodiments, wherein the coronavirus vaccine and/or the influenza vaccine retains at least 50%, 60%, or 70% of its original bioactivity (e.g., immunogenicity) after storage at about 45° C. for 2 weeks; at least 30%, 40%, or 50% of its original bioactivity (e.g., immunogenicity) after storage at about 45° C. for 4 weeks; at least 30%, 40%, or 50% of its original bioactivity (e.g., immunogenicity) after storage at about 45° C. for 8 weeks; and/or at least 30%, 40%, or 50% of its original bioactivity (e.g., immunogenicity) after storage at about 45° C. for 12 weeks.
E81. The microneedle device, the plurality of microneedles, or the microneedle of any one of the preceding embodiments, wherein the immune response is a humoral immune response comprising:
   (i) an elevated coronavirus-specific antibody titer (e.g., a SARS-CoV-2-specific antibody, e.g., an antibody specific to a SARS-CoV-2 spike protein, or a subunit thereof) detectable in the blood of the subject, e.g., detectable at least three, four, five, or six days, or at least 1, 2, 3, 4, 5, 6, 7, 8, 9, 10, 11, 12, 13, 14, 15, and/or 16-weeks post immunization; and/or
   (ii) an elevated anti-coronavirus IgG (e.g., anti-SARS-CoV-2 IgG) titer detectable in the blood of the subject, e.g., detectable at least 4, 5, or 6 days, or at least 1, 2, 3, or 4 weeks, or at least 1, 2, 3, 4, 5, and/or 6-months, post immunization.
E82. The microneedle device, the plurality of microneedles, or the microneedle of any one of the preceding embodiments, wherein an elevated coronavirus-specific antibody titer (e.g., a SARS-CoV-2-specific antibody, e.g., an antibody specific to a SARS-CoV-2 spike protein, or a subunit thereof) is detectable in the blood of the subject for the duration of a complete coronavirus season post-immunization.
E83. The microneedle device, the plurality of microneedles, or the microneedle of any one of the preceding embodiments, wherein the immune response is a cellular immune response comprising an increase in the level of coronavirus-specific antibody-secreting cells, e.g., SARS-CoV-2-specific antibody secreting cells, in the blood of the subject, e.g., at least 1, 2, 3, 4, 5, 6, 7, 8, 9, 10, 11, and/or 12-weeks post immunization.
E84. The microneedle device, the plurality of microneedles, or the microneedle of any one of the preceding embodiments, wherein immune response is a humoral immune response comprising:
   (i) an elevated hemagglutination inhibition (HAI) antibody titer detectable in the blood of the subject, e.g., detectable at least three, four, five, six days, or at least 1, 2, 3, 4, 5, 6, 7, 8, 9, 10, 11, 12, 13, 14, 15, and/or 16-weeks post immunization; and/or
   (ii) an elevated anti-influenza IgG titer detectable in the blood of the subject, e.g., detectable at least 4, 5, or 6 days, or at least 1, 2, 3, or 4 weeks, or at least 1, 2, 3, 4, 5, and/or 6-months, post immunization.
E85. The microneedle device, the plurality of microneedles, or the microneedle of any one of the preceding embodiments, wherein an elevated hemagglutination inhibition (HAI) antibody titer is detectable in the blood of the subject for the duration of a complete flu season post immunization.
E86. The microneedle device, the plurality of microneedles, or the microneedle of any one of the preceding embodiments, wherein immune response is a cellular immune response comprising an increase in the level of IFNγ secreting cell in the blood of the subject, e.g., at least 1, 2, 3, 4, 5, 6, 7, 8, 9, 10, 11, and/or 12-weeks post immunization.
E87. The microneedle device, the plurality of microneedles, or the microneedle of any one of the preceding embodiments, wherein immune response is a cellular immune response comprising an increase in the production of IFNγ per a preselected number of cells in the blood of the subject, e.g., at least 1, 2, 3, 4, 5, 6, 7, 8, 9, 10, 11, and/or 12-weeks post immunization.
E88. The microneedle device, the plurality of microneedles, or the microneedle of any one of the preceding embodiments, wherein the microneedles comprise a dose, e.g., a standard dose, of the coronavirus vaccine and/or the influenza vaccine.
E89. The microneedle device, the plurality of microneedles, or the microneedle of embodiment E88, wherein the dose of the coronavirus vaccine comprises between about 0.5 μg and about 500 μg (e.g., between about 1 and about 400 μg, between about 1 and about 300 μg, between about 1 and about 250 μg, between about 5 and about 200 μg, between about 10 and about 150 μg, between about 1 and about 50 μg, between about 1 and about 25 μg, between about 1 and about 15 μg, between about 10 and about 100 μg, between about 20 and about 80 μg, between about 40 and about 70 μg, between about 100 and about 150 μg, or between about 150 and about 200 μg).
E90. The microneedle device, the plurality of microneedles, or the microneedle of embodiment E88 or E89, wherein the dose of the coronavirus vaccine comprises at least about 0.5 μg (e.g., about 0.5, 1, 2, 3, 4, 5, 6, 7, 8, 9, 10, 12, 14, 16, 18, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 325, 350, 375, 400, 425, 450, 475, 500, 550, 600, 650, 700, 750, 800, 850, 900, 950, or 1000 μg).
E91. The microneedle device, the plurality of microneedles, or the microneedle of any one of the preceding embodiments, wherein the microneedles comprise at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25% or more of a dose, e.g., a standard dose, of the coronavirus vaccine.
E92. The microneedle device, the plurality of microneedles, or the microneedle of any one of the preceding embodiments, wherein each microneedle comprises between about 0.002 μg and about 5 μg of the coronavirus vaccine (e.g., at least about 0.003 μg, 0.004 μg, 0.005 μg, 0.01 μg, 0.02 μg, 0.03 μg, 0.04 μg, 0.05 μg, 0.06 μg, 0.07 μg, 0.08 μg, 0.09 μg, 0.1 μg, 0.12 μg, 0.14 μg, 0.16 μg, 0.18 μg, 0.2 μg, 0.25 μg, 0.3 μg, 0.35 μg, 0.4 μg, 0.45 μg, 0.5 μg, 0.6 μg, 0.7 μg, 0.8 μg, 0.9 μg, 1.0 μg, 1.2 μg, 1.4 μg, 1.6 μg, 1.8 μg, 2.0 μg, 2.5 μg, 3.0 μg, 3.5 μg, 4.0 μg, 4.5 μg, or 5 μg of the coronavirus vaccine).
E93. The microneedle device, the plurality of microneedles, or the microneedle of embodiment E88, wherein the standard dose of the influenza vaccine comprises between about 0.5 μg and about 65 μg per strain (e.g., about 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, or 65 μg per strain).
E94. The microneedle device, the plurality of microneedles, or the microneedle of embodiment E88, wherein the standard dose of the influenza vaccine comprises about 15 μg per strain.
E95. The microneedle device, the plurality of microneedles, or the microneedle of any one of the preceding embodiments, wherein the plurality of microneedles comprises at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25% or more of the standard dose of the coronavirus vaccine and/or the influenza vaccine.
E96. The microneedle device, the plurality of microneedles, or the microneedle of any one of embodiments E1-E5, E7, or E10-E95, wherein the plurality comprises about 0.1 μg to about 65 μg of influenza vaccine (e.g., about 0.1 μg, about 0.2 μg, about 0.3 μg, about 0.4 μg, about 0.5 μg, about 0.6 μg, about 0.7 μg, about 0.8 μg, about 0.9 μg, about 1 μg, about 1 μg to about 10 μg, about 10 μg to about 20 μg, about 20 μg to about 30 μg, about 30 μg to about 40 μg, about 40 μg to about 50 μg, about 50 μg to about 65 μg of influenza vaccine).
E97. The microneedle device, the plurality of microneedles, or the microneedle of any one of the preceding embodiments, wherein the microneedle comprises an implantable sustained-release tip applied to a dissolvable base.
E98. The microneedle device, the plurality of microneedles, or the microneedle of any one of the preceding embodiments, wherein the backing is chosen from a solid support, e.g., a paper-based material, a plastic material, a polymeric material, or a polyester-based material (e.g., a Whatman 903 paper, a polymeric tape, a plastic tape, an adhesive-backed polyester tape, or other medical tape).
E98a. The microneedle device, the plurality of microneedles, or the microneedle of any one of the preceding embodiments, wherein the backing comprises an adhesive, e.g., an adhesive comprising (e.g., impregnating) a solid support (e.g., a porous support matrix), or an adhesive without a solid support.
E98b. The microneedle device, the plurality of microneedles, or the microneedle of any one of the preceding embodiments, wherein the backing comprises an adhesive that is treated, e.g., by temperature, oxidation, and/or UV irradiation.
E98c. The microneedle device, the plurality of microneedles, or the microneedle of any one of the preceding embodiments, wherein the backing comprises an adhesive that provides a connection to a solid support (e.g., a porous support matrix).
E99. The microneedle device, the plurality of microneedles, or the microneedle of any one of the preceding embodiments, wherein the microneedle tip (e.g., silk fibroin tip) comprises silk fibroin at a concentration of about 1% w/v to about 10% w/v (e.g., about 1, 2, 3, 4, 5, 6, 7, 8, 9, or 10% w/v, or a silk fibroin having a molecular weight distribution according to FIG. 4, or, comprises silk fibroin in an amount between about 20 μg to about 245 μg, e.g., per 121 microneedle array).
E100. The microneedle device, the plurality of microneedles, or the microneedle of any one of the preceding embodiments, wherein the microneedle tip (e.g., silk fibroin tip) comprises about 1% w/v to about 10% w/v (e.g., about 1, 2, 3, 4, 5, 6, 7, 8, 9, or 10% w/v) of 10 MB silk fibroin solution, or a silk fibroin solution according to FIG. 4.
E101. The microneedle device, the plurality of microneedles, or the microneedle of any one of the preceding embodiments, wherein the microneedle tip (e.g., silk fibroin tip) comprises about 1% w/v to about 10% w/v (e.g., about 1, 2, 3, 4, 5, 6, 7, 8, 9, or 10% w/v) of 60 MB silk fibroin solution, or a silk fibroin solution according to FIG. 4, e.g., a 100 kDa to 200 kDa (e.g., about 153 kDa) silk fibroin solution.
E102. The microneedle device, the plurality of microneedles, or the microneedle of any one of the preceding embodiments, wherein the microneedle tip (e.g., silk fibroin tip) comprises about 1% w/v to about 10% w/v (e.g., about 1, 2, 3, 4, 5, 6, 7, 8, 9, or 10% w/v) of 120 MB silk fibroin solution, or a silk fibroin solution according to FIG. 4, e.g., a 70 kDa to 150 kDa (e.g., about 100 kDa) silk fibroin solution.
E103. The microneedle device, the plurality of microneedles, or the microneedle of any one of the preceding embodiments, wherein the microneedle tip (e.g., silk fibroin tip) comprises about 1% w/v to about 10% w/v (e.g., about 1, 2, 3, 4, 5, 6, 7, 8, 9, or 10% w/v) of 180 MB silk fibroin solution, or a silk fibroin solution according to FIG. 4, e.g., a 36 kDa to 100 kDa (e.g., about 71 kDa) silk fibroin solution.
E104. The microneedle device, the plurality of microneedles, or the microneedle of any one of the preceding embodiments, wherein the microneedle tip (e.g., silk fibroin tip) comprises about 1% w/v to about 10% w/v (e.g., about 1, 2, 3, 4, 5, 6, 7, 8, 9, or 10% w/v) of 480 MB silk fibroin solution, or a silk fibroin solution according to FIG. 4, e.g., a 1 kDa to 60 kDa (e.g., about 16 kDa) silk fibroin solution.
E105. The microneedle device, the plurality of microneedles, or the microneedle of any one of the preceding embodiments, wherein the microneedle tip (e.g., silk fibroin tip) is configured for sustained-release and comprises between about 1% to about 10% w/v 10 MB silk fibroin solution.
E106. The microneedle device, the plurality of microneedles, or the microneedle of any one of the preceding embodiments, wherein the microneedle tip (e.g., silk fibroin tip) is configured for sustained-release and comprises between about 1% to about 10% w/v 60 MB silk fibroin solution.
E107. The microneedle device, the plurality of microneedles, or the microneedle of any one of the preceding embodiments, wherein the microneedle tip (e.g., silk fibroin tip) is configured for sustained-release and comprises between about 1% to about 10% w/v 120 MB silk fibroin solution.
E108. The microneedle device, the plurality of microneedles, or the microneedle of any one of the preceding embodiments, wherein the microneedle tip (e.g., silk fibroin tip) is configured for sustained-release and comprises between about 1% to about 10% w/v 180 MB silk fibroin solution.
E109. The microneedle device, the plurality of microneedles, or the microneedle of any one of the preceding embodiments, wherein the microneedle tip (e.g., silk fibroin tip) is configured for sustained-release and comprises between about 1% to about 10% w/v 480 MB silk fibroin solution.
E110. The microneedle device, the plurality of microneedles, or the microneedle of any one of the preceding embodiments, wherein the base (e.g., dissolvable base) comprises two or more of:
   (i) a polysaccharide (e.g., dextran);
   (ii) a disaccharide (e.g., sucrose, maltose, and trehalose);
   (iii) a polymer (e.g., methyl cellulose, polyethylene glycol (PEG), carboxymethylcellulose (CMC), polyvinylpyrrolidone (PVP), polyvinyl alcohol (PVA), and hyaluronate);
   (iv) a protein (e.g., gelatin);
   (v) a plasticizer (e.g., glycerol, propanediol); and
   (vi) a surfactant (e.g., an octyl phenol ethoxylate (e.g., Triton-X), a polysorbate, a poloxamers, and/or a polyethoxylated alcohol).
E111. The microneedle device, the plurality of microneedles, or the microneedle of any one of the preceding embodiments, wherein the base comprises one or more of gelatin, dextran, glycerol, polyethylene glycol (PEG) (e.g., including low molecular weight PEG), sucrose, trehalose, maltose, carboxymethylcellulose (CMC), polyvinylpyrrolidone (PVP), polyvinyl alcohol (PVA), hyaluronate, methyl cellulose, and/or a surfactant (e.g., a octyl phenol ethoxylate (e.g., Triton-X), polysorbate, poloxamers, such as P188, and/or a polyethoxylated alcohol), optionally wherein the microneedle is configured for sustained release.
E112. The microneedle device, the plurality of microneedles, or the microneedle of any one of the preceding embodiments, wherein the base comprises dextran, sucrose, glycerol, and a surfactant, optionally configured for sustained release.
E113. The microneedle device, the plurality of microneedles, or the microneedle of any one of the preceding embodiments, which is configured for sustained release comprises:
   (i) between about 20% to about 40%, e.g., 30%, 70 kDa dextran;
   (ii) between about 5% to about 15%, e.g., about 10%, sucrose;
   (iii) between about 0.5% to about 2.5%, e.g., about 1%, glycerol; and
   (iv) between about 0.001% to about 1%, e.g., about 0.01%, Triton-X; (optionally wherein this is the solution used for casting and/or used for the composition of the dried, solidified base)
E114. The microneedle device, the plurality of microneedles, or the microneedle of any one of embodiments E110-E113, wherein the dextran has a molecular weight of between about 30 kD and about 600 kDa.
E115. The microneedle device, the plurality of microneedles, or the microneedle of any one of embodiments E110-E114, wherein the dextran is derived from Leuconostoc mesenteroides.
E116. The microneedle device, the plurality of microneedles, or the microneedle of any one of the preceding embodiments, wherein the base does not comprise poly(acrylic acid) (PAA).
E117. The microneedle device, the plurality of microneedles, or the microneedle of any one of the preceding embodiments, wherein the microneedle tip (e.g., silk fibroin tip) comprises:
   (i) a disaccharide (e.g., sucrose, maltose, and trehalose);
   (ii) a polymer (e.g., methyl cellulose, polyethylene glycol (PEG), carboxymethylcellulose (CMC), polyvinylpyrrolidone (PVP), polyvinyl alcohol (PVA), hyaluronate);
   (iii) an amino acid (e.g., threonine);
   (iv) a plasticizer (e.g., glycerol, propanediol);
   (v) a buffer (e.g., PBS);
   (vi) a surfactant (e.g., an octyl phenol ethoxylate (e.g., Triton-X), a polysorbate (e.g., Tween 20), a poloxamers, and/or a polyethoxylated alcohol); and/or
   (vii) an adjuvant.
E118. The microneedle device, the plurality of microneedles, or the microneedle of any one of the preceding embodiments, wherein the microneedle tip (e.g., silk fibroin tip) comprises an excipient (e.g., an excipient described herein).
E119. The microneedle device, the plurality of microneedles, or the microneedle of any one of the preceding embodiments, wherein the microneedle tip (e.g., silk fibroin tip) comprises an adjuvant (e.g., an adjuvant described herein).
E120. The microneedle device, the plurality of microneedles, or the microneedle of any one of the preceding embodiments, wherein the microneedle tip (e.g., silk fibroin tip) comprises one or more of carboxymethylcellulose (CMC), sucrose, and threonine.
E121. The microneedle device, the plurality of microneedles, or the microneedle of any one of the preceding embodiments, wherein the microneedle tip (e.g., silk fibroin tip) comprises a buffer, optionally phosphate buffered saline (PBS).
E122. The microneedle device, the plurality of microneedles, or the microneedle of any one of the preceding embodiments, wherein the microneedle (e.g., the microneedle tip and/or the base) does not comprise carboxymethyl cellulose, or if CMC is present, it is present at an amount of 35% w/w or less.
E123. The microneedle device, the plurality of microneedles, or the microneedle of any one of the preceding embodiments, wherein the microneedle (e.g., the microneedle tip) comprises less than 35% w/w of carboxymethyl cellulose (e.g., less than 30% w/w, 29% w/w, 28% w/w, 27% w/w, 26% w/w, 25% w/w, 24% w/w, 23% w/w, 22% w/w/, 21% w/w, 20% w/w, 19% w/w, 18% w/w, 17% w/w, 16% w/w, 15% w/w, 14% w/w, 13% w/w, 12% w/w, 11% w/w, 10% w/w, 9% w/w, 8% w/w, 7% w/w, 6% w/w, 5% w/w, 4% w/w, 3% w/w, 2% w/w, 1% w/w, 0.5% w/w, 0.4% w/w, 0.3% w/w, 0.2% w/w, or 0.1% w/w of carboxymethyl cellulose).
E124. The microneedle device, the plurality of microneedles, or the microneedle of any one of the preceding embodiments, which comprises substantially only dissolvable materials, e.g., substantially only polymeric-based and/or or sugar-based materials.
E125. The microneedle device, the plurality of microneedles, or the microneedle of any one of the preceding embodiments, which is configured to be applied to a biological barrier (e.g., skin) of a subject, and left in place to fully dissolve.
E126. The microneedle device, the plurality of microneedles, or the microneedle of any one of the preceding embodiments, wherein the microneedle is configured to implant a sustained-release tip into the skin of a subject, e.g., a human subject, at a depth (e.g., a max penetration depth of the distal part of tip) of between about 100 μm and about 1 mm (e.g., between about 100 μm and 600 μm).
E127. The microneedle device, the plurality of microneedles, or the microneedle of any one of the preceding embodiments, wherein the length of the microneedle is between about 350 μm to about 1500 μm.
E128. The microneedle device, the plurality of microneedles, or the microneedle of any one of the preceding embodiments, wherein the height of the microneedle tip (e.g., silk fibroin tip) may extend to approximately half of the full height of the microneedle.
E129. The microneedle device, the plurality of microneedles, or the microneedle of any one of the preceding embodiments, wherein the height of the microneedle tip (e.g., silk fibroin tip) is between about 75 μm to about 475 μm.
E130. The microneedle device, the plurality of microneedles, or the microneedle of any one of the preceding embodiments, wherein the microneedle tip (e.g., silk fibroin tip) comprises a tip radius between about 0.5 m to about 100 m.
E131. The microneedle device, the plurality of microneedles, or the microneedle of any one of the preceding embodiments, wherein the microneedle tip (e.g., silk fibroin tip) comprises a tip radius between about 5 m to about 10 m.
E132. The microneedle device, the plurality of microneedles, of the microneedle of any one of the preceding embodiments, wherein the microneedle tip (e.g., silk fibroin tip) comprises an angle between about 5 degrees and about 45 degrees.
E133. A method of providing immunity to a virus (e.g., SARS-CoV-2, SARS-CoV, MERS-CoV, and/or influenza), e.g., broad spectrum immunity, in a subject comprising contacting the skin of the subject with the microneedle device, the plurality of microneedles, or the microneedle of any one of the preceding embodiments.
E134. A method of providing a controlled- or sustained-release of an antigen, e.g., a coronavirus vaccine and/or an influenza vaccine (e.g., a SARS-CoV-2 antigen, a SARS-CoV antigen, a MERS-CoV antigen, and/or an influenza antigen, or a vaccine preparation thereof), in a subject comprising contacting the skin of the subject with the microneedle device, the plurality of microneedles, or the microneedle of any one of embodiments E1-132.
E135. A method of enhancing an immune response to a virus (e.g., a coronavirus (e.g., SARS-CoV-2, SARS-CoV, and/or MERS-CoV) and/or an influenza virus) in a subject comprising contacting the skin of the subject with the microneedle device, the plurality of microneedles, or the microneedle of any one of embodiments E1-132.
E136. The method of any one of embodiments E133-E135, wherein the immune response is a humoral and/or cellular immune response comprising:
   (i) an elevated coronavirus-specific antibody titer (e.g., a SARS-CoV-2-specific antibody, e.g., an antibody specific to a SARS-CoV-2 spike protein, or a subunit thereof) detectable in the blood of the subject, e.g., detectable at least three, four, five, six days, or at least 1, 2, 3, 4, 5, 6, 7, 8, 9, 10, 11, 12, 13, 14, 15, and/or 16-weeks post immunization;
   (ii) an elevated anti-coronavirus IgG (e.g., anti-SARS-CoV-2 IgG) titer detectable in the blood of the subject, e.g., detectable at least 4, 5, or 6 days, or at least 1, 2, 3, or 4 weeks, or at least 1, 2, 3, 4, 5, and/or 6-months, post immunization;
   (iii) an elevated coronavirus-specific antibody titer (e.g., a SARS-CoV-2-specific antibody, e.g., an antibody specific to a SARS-CoV-2 spike protein, or a subunit thereof) is detectable in the blood of the subject for the duration of a complete coronavirus season post immunization;
   (iv) an increase in the level of IFNγ secreting cell in the blood of the subject, e.g., at least 1, 2, 3, 4, 5, 6, 7, 8, 9, 10, 11, and/or 12-weeks post immunization; and/or
   (v) an increase in the production of IFNγ per a preselected number of cells in the blood of the subject, e.g., at least 1, 2, 3, 4, 5, 6, 7, 8, 9, 10, 11, and/or 12-weeks post immunization.
E137. The method of any one of embodiments E133-E136, wherein the immune response is a humoral and/or cellular immune response comprising:
   (i) an elevated hemagglutination inhibition (HAI) antibody titer detectable in the blood of the subject, e.g., detectable at least three, four, five, six days, or at least 1, 2, 3, 4, 5, 6, 7, 8, 9, 10, 11, 12, 13, 14, 15, and/or 16-weeks post immunization;
   (ii) an elevated anti-influenza IgG titer detectable in the blood of the subject, e.g., detectable at least 4, 5, or 6 days, or at least 1, 2, 3, or 4 weeks, or at least 1, 2, 3, 4, 5, and/or 6-months, post immunization.
   (iii) an elevated hemagglutination inhibition (HAI) antibody titer is detectable in the blood of the subject for the duration of a complete flu season post-immunization;
   (iv) an increase in the level of IFNγ secreting cell in the blood of the subject, e.g., at least 1, 2, 3, 4, 5, 6, 7, 8, 9, 10, 11, and/or 12-weeks post-immunization; and/or
   (v) an increase in the production of IFNγ per a preselected number of cells in the blood of the subject, e.g., at least 1, 2, 3, 4, 5, 6, 7, 8, 9, 10, 11, and/or 12-weeks post immunization.
E138. A method of providing broad-spectrum immunity to a coronavirus (e.g., SARS-CoV-2, SARS-CoV, and/or MERS-CoV) and/or an influenza virus, in a subject, said method comprising contacting the skin of the subject with the microneedle device, the plurality of microneedles, or the microneedle of any one of embodiments E1-E132, e.g., resulting in an immune response (e.g., a cellular immune response and/or a humoral immune response) to a drifted strain of the coronavirus and/or the influenza virus, in the subject.
E139. The method of any one of embodiments E133-E138, wherein the coronavirus vaccine and/or the influenza vaccine is administered in an amount (e.g., a dosage) and/or over a time period sufficient to result in one or more of:
   (i) exposure in the subject to one or more antigens in the coronavirus and/or influenza vaccine in an amount and/or period of time to result in broad spectrum immunity, e.g., to result in an immune response (e.g., a cellular immune response and/or a humoral immune response) to a drifted strain of the coronavirus and/or the influenza virus, in the subject; or
   (ii) a level of one or more antigens in the subject that is substantially steady, e.g., about 20%, 15%, 10%, 5%, or 1% to an amount, e.g., minimum amount, needed to result in an immune response (e.g., a cellular immune response and/or a humoral immune response) to the one or more antigens.
E140. The method of any one of embodiments E133-E139, wherein the coronavirus vaccine and influenza vaccine are administered in order to maintain a dosage of each vaccine (e.g., an antigen concentration) for a period of time sufficient to result in broad spectrum immunity, e.g., to result in an immune response (e.g., a cellular immune response and/or a humoral immune response) to a drifted strain of the coronavirus (e.g., a drifted SARS-CoV-2, SARS-CoV, and/or MERS-CoV virus) and/or a drifted strain of an influenza virus, in the subject (e.g., wherein the period of time is about 1 to 21 days, e.g., about 5 to 25 days or about 10 to 15 days, e.g., about 1, 2, 3, 4, 5, 6, 7, 8, 9, 10, 11, 12, 13, 14, 15, 16, 17, 18, 19, 20, 21, 22, 23, 24, or 25 days).
E141. The method of any one of embodiments E133-E140, wherein the microneedle device, the plurality of microneedles, or the microneedle maintains antigen release and/or level in the subject over a sustained period of time.
E142. The method of any one of embodiments E133-E141, wherein the microneedle device, the plurality of microneedles, or the microneedle maintains a continuous or non-continuous antigen release into the subject over a sustained period of time.
E143. The method of any one of embodiments E133-E142, wherein the coronavirus vaccine and/or influenza vaccine are administered, e.g., released by the microneedle device, the plurality of microneedles, or the microneedle, over a period of time comprising at least about one week, e.g., about 1-2 weeks, about 1-3 weeks, or about 1-4 weeks.
E144. The method of any one of embodiments E133-E143, wherein the coronavirus vaccine and/or influenza vaccine are administered, e.g., released by the microneedle device, the plurality of microneedles, or the microneedle, over a period of time comprising at least about 4 days (e.g., about 4, 5, 6, 7, 8, 9, 10, 11, 12, 13, 14, 15, 16, 17, 18, 19, 20, 21, 22, 23, 24, or 25 days, or more).
E145. The method of any one of embodiments E133-E144, wherein at least about 1% of the dosage of the coronavirus vaccine and/or the influenza vaccine (e.g., at least about 0.5% to about 10%, at least about 5% to about 15% at least about 10% to about 20% of the dosage), e.g., released by the microneedle device, the plurality of microneedles, or the microneedle, e.g., into the subject, is maintained over a period of time comprising at least about 4 days (e.g., about 4, 5, 6, 7, 8, 9, 10, 11, 12, 13, or 14 days, or more, e.g., between about 5 days and about 25 days, between about 10 days and 20 days, or between about 14 days and 15 days).
E146. The method of any one of embodiments E133-E145, wherein the coronavirus vaccine and/or influenza vaccine are administered, e.g., released by the microneedle device, the plurality of microneedles, or the microneedle, in a plurality of fractional doses of a total dose (e.g., a standard dose) over a time period, e.g., such that an immune response and/or broad-spectrum immunity is achieved, wherein the amount of the coronavirus vaccine and/or influenza vaccine administered in each of the fractional doses is no more than 1/X, wherein X is any number, e.g., wherein X is 0.1, 0.2, 0.3, 0.4, 0.5, 0.6, 0.7, 0.8, 0.9, 1, 2, 3, 4, 5, 6, 7, 8, 9, 10, 15, 20, 30, 40, 50, 60, 70, 80, 90, or 100 or more, of the total dose (e.g., a standard dose) of the vaccine.
E147. The method of any one of embodiments E133-E146, wherein the coronavirus vaccine and/or influenza vaccine is administered, e.g., released by the microneedle device, the plurality of microneedles, or the microneedle, e.g., into the skin of the subject, in a plurality of doses equivalent to a percentage of a total dose (e.g., a percentage of a standard dose) over a time period, e.g., such that broad-spectrum immunity is achieved, wherein the amount of the coronavirus vaccine and/or influenza vaccine administered in each of the plurality of doses is about X %, wherein X is any number, e.g., wherein X is 0.1, 0.2, 0.3, 0.4, 0.5, 0.6, 0.7, 0.8, 0.9, 1, 2, 3, 4, 5, 6, 7, 8, 9, 10, 15, 20, 30, 40, 50, 60, 70, 80, 90, 100, 125, 150, 175, 200, 300, 400, or 500 or more, of the total dose (e.g., a standard dose) of the vaccine.
E148. The method of any one of embodiments E133-E147, wherein the coronavirus vaccine and/or influenza vaccine is administered such that broad-spectrum immunity is achieved, e.g., such that an immune response, e.g., a cellular immune and/or humoral immune response to a drifted strain (e.g., a drifted coronavirus strain, e.g., a drifted SARS-CoV-2 strain, and/or a drifted influenza strain) is achieved.
E149. The method of any one of embodiments E133-E148, wherein the coronavirus vaccine and/or influenza vaccine is administered as two, three, four, five, six, seven, eight, nine, ten or more fractional doses.
E150. The method of any one of embodiments E133-E149, wherein the total dose (e.g., the standard dose) of the coronavirus vaccine and/or influenza vaccine is administered to achieve broad-spectrum immunity.
E151. The method of any one of embodiments E133-E149, wherein less than the total dose (e.g., the standard dose) of the coronavirus vaccine and/or influenza vaccine is administered to achieve broad-spectrum immunity.
E152. The method of any one of embodiments E133-E149, wherein more than the total dose (e.g., the standard dose) of the coronavirus vaccine and/or influenza vaccine is administered to achieve broad-spectrum immunity.
E153. The method of any one of embodiments E146-E149, wherein the amount of the coronavirus vaccine and/or influenza vaccine administered in each of the fractional doses is the same.
E154. The method of any one of embodiments E146-E149, wherein the amount of the coronavirus vaccine and/or influenza vaccine administered in each of the fractional doses is different.
E155. The method of any one of embodiments E146-E154, wherein the plurality of fractional doses is administered by intramuscular injection or intradermal injection, e.g., to achieve controlled- or sustained-release of the coronavirus vaccine and/or influenza vaccine.
E156. The method of any one of any one of embodiments E146-E155, wherein each dose of the plurality of fractional doses is administered at least once or twice a day, at least once every two days, at least once every three days, at least once every four days, at least once every five days, at least once every 6 days, at least one a week, or at least once a month for the duration of the time period.
E157. The method of any one of embodiments E133-E156, wherein:
   (i) the coronavirus vaccine comprises an antigen associated with a first coronavirus strain, and administration of a dose of the coronavirus vaccine to the subject results in broad-spectrum immunity to a second coronavirus strain (e.g., a drifted SARS-CoV-2 strain) not present in, or associated with, the composition or the vaccine;
   (ii) the influenza vaccine comprises a first influenza strain and administration of a dose of the first influenza strain to the subject results in broad-spectrum immunity to a second influenza strain (e.g., a drifted influenza strain) not present in the composition or the vaccine;
   (ii) the influenza vaccine comprises a first influenza A strain and administration of a dose of the first influenza A strain to the subject results in broad-spectrum immunity to a drifted influenza strain (e.g., a drifted influenza A, B, C, and/or D strain) not present in the composition or the vaccine;
   (iii) the influenza vaccine comprises a first influenza B strain and administration of a dose of the first influenza B strain to the subject results in broad-spectrum immunity to a drifted influenza strain (e.g., a drifted influenza A, B, C, and/or D strain) not present in the composition or the vaccine;
   (iv) the influenza vaccine comprises a first influenza C strain and administration of a dose of the first influenza C strain to the subject results in broad-spectrum immunity to a drifted influenza strain (e.g., a drifted influenza A, B, C, and/or D strain) not present in the composition or the vaccine; and/or
   (v) the influenza vaccine comprises a first influenza D strain and administration of a dose of the first influenza D strain to the subject results in broad-spectrum immunity to a drifted influenza strain (e.g., a drifted influenza A, B, C, and/or D strain) not present in the composition or the vaccine.
E158. The method of embodiment E157, wherein the first influenza A vaccine comprises:
   (i) an H1N1 (e.g., A/Michigan and/or A/California) vaccine; and/or
   (ii) an H3N2 (e.g., A/Hong Kong and/or A/Switzerland) vaccine.
E159. The method of embodiment E157 or E158, wherein the drifted influenza A strain comprises:
   (i) an H1N1 strain (e.g., A/Michigan and/or A/California); and/or
   (ii) an H3N2 strain (e.g., A/Hong Kong and/or A/Switzerland).
E160. The method of any one of embodiments E157-159, wherein:
   (i) the first influenza A vaccine comprises an H1N1 vaccine to A/Michigan and the drifted influenza A strain comprises A/California; and/or
   (ii) the first influenza A vaccine comprises an H3N2 vaccine to A/Hong Kong and the drifted influenza A strain is A/Switzerland.
E161. The method of any one of embodiments E157-160, wherein the first influenza B vaccine comprises:
   (i) a B/Yamagata lineage strain (e.g., B/Phuket); and/or
   (ii) a B/Victoria lineage strain (e.g., B/Brisbane).
E162. The method of any one of embodiments E157-161, wherein:
   (i) the drifted influenza B strain is a B/Yamagata lineage strain (e.g., B/Phuket); and/or
   (ii) the drifted influenza B strain is a B/Victoria lineage strain (e.g., B/Brisbane).
E163. The method of any one of embodiments E157-162, wherein the first influenza B vaccine is to the B/Victoria lineage strain B/Brisbane and the drifted influenza B strain is the B/Yamagata lineage strain B/Phuket.
E164. The method of any one of embodiments E133-163, wherein the immune response and/or broad-spectrum immunity comprises a cellular and/or humoral immune response comprising:
   (i) an elevated coronavirus-specific antibody titer (e.g., a SARS-CoV-2-specific antibody, e.g., an antibody specific to a SARS-CoV-2 spike protein, or a subunit thereof) detectable in the blood of the subject, e.g., detectable at least 4, 5, or 6 days, or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 51, and/or 52-weeks or more post immunization, optionally wherein the elevated coronavirus-specific antibody titer is to a drifted coronavirus strain (e.g., a drifted SARS-CoV-2 strain);
   (ii) an elevated anti-coronavirus IgG (e.g., anti-SARS-CoV-2 IgG) titer detectable in the blood of the subject, e.g., detectable at least 4, 5, or 6 days, or at least 1, 2, 3, or 4 weeks, or at least 1, 2, 3, 4, 5, 6, 7, 8, 9, 10, 11, and/or 12-months or more post immunization, optionally wherein the elevated anti-coronavirus IgG titer is to a drifted coronavirus strain (e.g., a drifted SARS-CoV-2 strain); and/or
   (iii) a level of antibody secreting plasma cells (ASC) against the coronavirus, e.g., the SARS-CoV-2 virus, e.g., the drifted SARS-CoV-2 strain, detectable in the bone marrow of the subject, e.g., detectable at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 51, and/or 52-weeks or more post-immunization.
E165. The method of any one of embodiments E133-164, wherein an elevated coronavirus-specific antibody titer (e.g., a SARS-CoV-2-specific antibody, e.g., an antibody specific to a SARS-CoV-2 spike protein, or a subunit thereof) is detectable in the blood of the subject for the duration of a complete coronavirus season post immunization, optionally wherein the elevated coronavirus-specific antibody titer is to a drifted coronavirus strain (e.g., a drifted SARS-CoV-2 strain).
E166. The method of any one of embodiments E133-165, wherein the percent seroconversion, e.g., based on the elevated coronavirus-specific antibody titer (e.g., a SARS-CoV-2-specific antibody, e.g., an antibody specific to a SARS-CoV-2 spike protein, or a subunit thereof) detectable in the blood of the subject, e.g., at 6-month post immunization is greater than about 20% (e.g., 20%, 25%, 30%, 35%, 40%, 45%, 50%, 55%, 60%, 65%, 70%, 75%, 80%, 85%, 90%, or 95% or more, e.g., 100%).
E167. The method of embodiment E165, wherein the elevated coronavirus-specific antibody titer (e.g., a SARS-CoV-2-specific antibody, e.g., an antibody specific to a SARS-CoV-2 spike protein, or a subunit thereof), the elevated anti-coronavirus IgG (e.g., anti-SARS-CoV-2 IgG) titer, and/or the level of antibody secreting plasma cells (ASC) against the coronavirus (e.g., SARS-CoV-2 virus), is greater (e.g., 1-fold, 2-fold, 3-fold, 4-fold, 5-fold, 6-fold, 7-fold, 8-fold, 9-fold, 10-fold, 11-fold, 12-fold, 13-fold, 14-fold, or 15-fold or more greater) as compared to the administration of a single-dose or a bolus administration of the coronavirus vaccine (e.g., SARS-CoV-2 vaccine).
E168. The method of any one of embodiments E133-167, wherein the immune response and/or broad-spectrum immunity comprises a cellular and/or humoral immune response comprising:
   (i) an elevated hemagglutination inhibition (HAI) antibody titer detectable in the blood of the subject, e.g., detectable at least three, four, five, or six days, or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 51, and/or 52-weeks or more post immunization, optionally wherein the elevated HAI antibody titer is to a drifted influenza A, B, C, and/or D strain;
   (ii) an elevated anti-influenza IgG titer detectable in the blood of the subject, e.g., detectable at least 4, 5, or 6 days, or at least 1, 2, 3, or 4 weeks, or at least 1, 2, 3, 4, 5, 6, 7, 8, 9, 10, 11, and/or 12-months or more post immunization, optionally wherein the elevated anti-influenza IgG titer is to a drifted influenza A, B, C, and/or D strain; and/or
   (iii) a level of antibody secreting plasma cells (ASC) against the influenza virus, e.g., the drifted influenza A, B, C, and/or D strain, detectable in the bone marrow of the subject, e.g., detectable at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 51, and/or 52-weeks or more post-immunization.
E169. The method of embodiment E168, wherein an elevated hemagglutination inhibition (HAI) antibody titer is detectable in the blood of the subject for the duration of a complete flu season post immunization, optionally wherein the elevated HAI antibody titer is to a drifted influenza A, B, C, and/or D strain.
E170. The method of any one of embodiments E133-E169, wherein the percent seroconversion, e.g., based on the elevated HAI antibody titer detectable in the blood of the subject, e.g., at 6-month post immunization is greater than about 20% (e.g., 20%, 25%, 30%, 35%, 40%, 45%, 50%, 55%, 60%, 65%, 70%, 75%, 80%, 85%, 90%, or 95% or more, e.g., 100%).
E171. The method of any one of embodiments E133-E170, wherein broad-spectrum immunity comprises a cellular immune response comprising an increase in the level of IFNγ secreting cell in the blood of the subject, e.g., at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 51, and/or 52-weeks or more post immunization.
E172. The method of any one of embodiments E133-E171, wherein broad-spectrum immunity comprises a cellular immune response comprising an increase in the production of IFNγ per a preselected number of cells in the blood of the subject, e.g., at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 51, and/or 52-weeks or more post immunization.
E173. The method of any one of embodiments E168-E172, wherein the elevated HAI antibody titer, the elevated anti-influenza IgG titer, the level of antibody secreting plasma cells (ASC) against the virus, the level of IFNγ secreting cells, and/or the production of IFNγ per a preselected number of cells, detectable in the subject is greater (e.g., 1-fold, 2-fold, 3-fold, 4-fold, 5-fold, 6-fold, 7-fold, 8-fold, 9-fold, 10-fold, 11-fold, 12-fold, 13-fold, 14-fold, or 15-fold or more greater) as compared to the administration of a single-dose or a bolus administration of the vaccine.
E174. A method for providing an immune response (e.g., a cellular immune response and/or a humoral immune response) and/or a broad spectrum immunity to a coronavirus (e.g., SARS-CoV-2, SARS-CoV, and/or MERS-CoV) and/or an influenza virus, in a subject, said method comprising contacting the skin of the subject with the microneedle device, the plurality of microneedles, or the microneedle of any one of embodiments E1-E132, wherein the coronavirus vaccine and/or influenza vaccine are administered in an amount (e.g., a dosage), and/or over a period of time comprising about 5-25 days (e.g., about 10-20 days, about 12-18 days, or about 14-15 days), e.g., about 5, 6, 7, 8, 9, 10, 11, 12, 13, 14, 15, 16, 17, 18, 19, 20, 21, 22, 23, 24, or 25 days, sufficient to elicit an immune response (e.g., a cellular immune response and/or a humoral immune response) to the coronavirus and/or the influenza virus, in the subject.
E175. The method of any one of embodiments E133-E174, wherein the subject (e.g., the human subject) is a pediatric subject.
E176. The method of any one of embodiments E133-E174, wherein the subject (e.g., the human subject) is an adult subject.
E177. The method of any one of embodiments E133-E174, wherein the subject (e.g., the human subject) is an elderly subject.
E178. The method of any one of embodiments E133-E177, wherein the coronavirus vaccine and/or the influenza vaccine is administered prophylactically.
E179. The method of any one of embodiments E133-E178, wherein a single dose administration of the coronavirus vaccine and/or influenza vaccine provides protection against infection, e.g., by coronavirus and/or influenza virus, in the subject.
E180. The method of any of embodiments E133-E179, wherein the coronavirus vaccine and/or influenza vaccine is administered as a patch, e.g., a single patch.
E181. The method of embodiment E180, wherein the patch is administered to the subject by a health care professional (e.g., a doctor or nurse).
E182. The method of embodiment E180, wherein the patch is self-administered.
E182a. The method of any one of embodiments E180-E182, wherein the patch is administrated using an applicator.
E183. The method of any one of embodiments E180-E182a, wherein the subject wears the patch for a period of time of less than 1 hour, e.g., about 1 minute to about 45 minutes, about 2 minutes to about 30 minutes, about 5 minutes to about 15 minutes, e.g., about 5 minutes.
E184. The method of any one of embodiments E133-E183, wherein the coronavirus vaccine and/or the influenza vaccine is administered seasonally, e.g., once per coronavirus season or influenza season (e.g., once per year).
E185. The method of any one of embodiments E133-E184, wherein the coronavirus vaccine and/or the influenza vaccine is administered on a regular booster schedule, e.g., yearly.
E186. The method of any one of embodiments E133-E185, wherein the coronavirus vaccine and/or the influenza vaccine provides protection (e.g., prevention) against coronavirus disease 2019 (COVID-19), Severe Acute Respiratory Syndrome (SARS), Middle East Respiratory Syndrome (MERS), influenza, or a combination thereof, e.g., for the duration of a coronavirus and/or influenza season.
E187. The method of any one of embodiments E133-E186, wherein the coronavirus vaccine and/or the influenza vaccine provides protection (e.g., prevention) against COVID-19 and/or influenza.
E188. A method of producing a microneedle device, the method comprising:
   providing a mold including a mold body with an array of needle cavities having a predefined shape, e.g., pyramid-shaped and/or conical-shaped needle cavities, formed therein;
   filling tips of the needle cavities with a composition (e.g., a composition comprising a silk fibroin), a coronavirus antigen, e.g., a coronavirus vaccine (e.g., one or more of: a SARS-CoV-2 antigen, a SARS-CoV antigen, or a MERS-CoV antigen, or a vaccine preparation thereof), and/or an influenza antigen, e.g., an influenza vaccine (e.g., one or more influenza antigens, or a vaccine preparation thereof);
   drying the filled tips of the needle cavities to create microneedle tips (e.g., silk fibroin tips), and optionally annealing the microneedle tips;
   filling the needle cavities of the mold with a base (e.g., dissolvable base) solution;
   drying the base solution to create base layers for the microneedle tips (e.g., silk fibroin tips); and
   optionally applying a backing to the base layers to create a microneedle device.
E189. The method of embodiment E188, further comprising removing the microneedle device from the mold, optionally before applying the backing.
E190. The method of embodiment E188 or E189, wherein the microneedle device is removed by bending the mold away from the microneedle device.
E191. The method of any one of embodiments E188-E190, further comprising packaging microneedle devices in a container with low moisture vapor transmission rate with a desiccant to maintain between about 0% and about 50% (e.g., between about 0% and 10%, between about 10% and about 20%, between about 20% and about 30%, between about 30% and about 40%, or between about 40% and 50%, e.g., about 25%) relative humidity inside the package.
E192. The method of any one of embodiments E188-E191, wherein the composition (e.g., the composition comprising silk fibroin), coronavirus vaccine, and/or influenza vaccine solution is dispensed into each needle cavity in the mold via nanoliter printing.
E193. The method of any one of embodiments E188-E192, wherein filling the tips of the needle cavities comprises dispensing a solution, e.g., a silk fibroin, a coronavirus vaccine, and/or an influenza vaccine solution, into each needle cavity.
E194. The method of any one of embodiments E188-E193, wherein filling the tips of the needle cavities comprises dispensing a coronavirus vaccine solution into a first portion of one or more needle cavities, and dispensing a first influenza vaccine solution into a second portion of one or more needle cavities.
E195. The method of embodiment E194, wherein filling the tips of the needle cavities further comprises dispensing a second influenza vaccine solution into a third portion of one or more needle cavities.
E196. The method of embodiment E195, wherein filling the tips of the needle cavities further comprises dispensing a third influenza vaccine solution into a fourth portion of one or more needle cavities.
E197. The method of embodiment E196, wherein filling the tips of the needle cavities further comprises dispensing a fourth influenza vaccine solution into a fifth portion of one or more needle cavities.
E198. The method of any one of embodiments E194-E197, wherein the first, second, third, and/or fourth influenza vaccines comprise an influenza A vaccine (e.g., a H1N1 vaccine and/or a H3N2 vaccine) and/or an influenza B vaccine (e.g., a B/Yamagata lineage and/or a B/Victoria lineage vaccine).
E199. The method of any one of embodiments E194-E198, wherein the combination of the first, second, third, and/or fourth influenza vaccines comprise a quadrivalent influenza vaccine, and/or wherein the combination of the coronavirus vaccine and the first, second, third, and fourth influenza vaccines comprise a pentavalent vaccine.
E200. The method of any one of embodiments E188-E199, wherein filling the tips of the needle cavities provides a microneedle device comprising a 4:1 ratio of influenza vaccines to coronavirus vaccine.
E201. The method of any one of embodiments E188-E200, wherein drying the filled tips of the needle cavities includes a primary drying step and a secondary drying step.
E202. The method of any one of embodiments E188-E201, wherein drying the base (e.g., dissolvable base) solution includes subjecting the mold to a centrifuge at 3900 rpm for 2 minutes and topping off the needle cavities with 50 μL of base solution.
E203. The method of any one of embodiments E188-E202, wherein the base filling occurs by nanoliter (nL) dispending.
E204. The method of any one of embodiments E188-E203, further comprising an annealing step (e.g., before filling the base) after the filling the tips of the needle cavities.
E205. The method of any one of embodiments E188-E204, further comprising a water annealing step (e.g., before filling the base) after the filling the tips of the needle cavities.
E206. The method of any one of embodiments E188-E205, wherein the backing layer comprises one of a paper backing layer and an adhesive plastic tape.
E207. The microneedle, microneedle device, or method of any one of embodiments E1-E206, wherein the coronavirus vaccine and/or influenza vaccine is adjuvanted.
E208. The microneedle, microneedle device, or method of any one embodiments E1-E206, wherein the coronavirus vaccine and/or influenza vaccine is unadjuvanted.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic drawing showing an exemplary microneedle fabrication process in accordance with an example of the invention.

FIG. 2 illustrates an exemplary microneedle device having an array of microneedles applied to a backing or “handle” layer.

FIG. 3 illustrates an exemplary microneedle device comprising a plurality of microneedles having sufficient mechanical properties (e.g., strength) and suitable geometry (e.g. tip sharpness, tip included angle, length, and inter-needle spacing) to pierce a biological barrier (e.g., skin) to achieve a delivery of a vaccine, antigen, and/or immunogen (e.g., a coronavirus vaccine, an influenza vaccine, or a combination thereof) to a subject.

FIG. 4 illustrates various molecular weight profiles of exemplary silk fibroin solutions useful in fabricating a microneedle or microneedle device described herein.

FIG. 5 is an image showing an enlarged view of a portion of an exemplary microneedle device fabricated to include different formulations in different microneedles.

FIG. 6 shows a comparison of spike-specific IgG titers obtained from blood samples collected from Balb/c mice (n=5/group) that were immunized with either 1 μg or 5 μg SARS-CoV-2 recombinant protein vaccine twice at 4-week intervals via bolus intramuscular injection (IM), bolus intradermal injection (ID), or equivalent daily fractional doses ID across 10 days to simulate sustained release kinetics (10d SR). Reported as geometric mean±95% confidence interval; One-way ANOVA with Tukey's post hoc analysis on log transformed values * p<0.05, ** p<0.01, **** p<0.0001 (only key comparisons plotted).

FIG. 7 shows a comparison of spike-specific IgG titers obtained from blood samples collected from Balb/c mice (n=5/group) that were immunized with 5 μg SARS-CoV-2 recombinant protein vaccine twice at 4-week intervals via bolus intramuscular injection (IM; neat or concentrated vaccine) or via microneedle devices (MIMIX; concentrated vaccine). Reported as geometric mean±95% confidence interval; Two-way repeated measures ANOVA with Tukey's post hoc analysis on log transformed values * p<0.05, ** p<0.01, *** p<0.001.

FIG. 8 shows a comparison of spike-specific IgG titers obtained from blood samples collected from Balb/c mice (n=5/group) that were immunized with 5 μg, 10 μg, or 15 μg SARS-CoV-2 recombinant protein vaccine twice at 4-week intervals via bolus intramuscular injection (IM) or once via microneedle devices (MIMIX). Reported as geometric mean±95% confidence interval; Two-way ANOVA with Šidák's post hoc analysis on log transformed values * p<0.05, ** p<0.01, *** p<0.001, **** p<0.0001.

FIG. 9 shows a comparison of spike-specific IgG titers obtained from blood samples collected from Balb/c mice (n=5/group) that were immunized with 5 μg SARS-CoV-2 recombinant protein vaccine via microneedle devices (MIMIX) using patches that were stored at room temperature at 10% RH for 8 weeks or freshly manufactured patches. Reported as geometric mean±95% confidence interval; unpaired t-test on log transformed values; p=0.7915.

### DETAILED DESCRIPTION

Provided herein are microneedles (e.g., silk fibroin-based microneedles), and microneedle devices (e.g., silk fibroin-based microneedle devices), including microneedle patches, configured to incorporate and subsequently release (e.g., administer) an effective amount of therapeutic agent, such as a vaccine, antigen, and/or immunogen (e.g., a coronavirus vaccine, an influenza vaccine, or a combination thereof) into a subject (e.g., into and/or across a biological barrier, such as the skin, of a subject). In certain embodiments, the microneedles and devices described herein are configured to encapsulate and release a nucleic acid molecule, such as an mRNA. In some embodiments, the mRNA encodes a vaccine antigen. Use of the microneedles and microneedle devices, including microneedle patches, described herein, can result in immunity (e.g., broad spectrum immunity) to a virus (e.g., a coronavirus and/or an influenza virus) in a subject.

The present disclosure is based, at least in part, on the discovery that modulating the kinetics of antigen presentation to mimic that of a natural infection (e.g., a viral infection) can drive a more potent immune response, such as a more potent cellular and/or humoral immune response (see, e.g., Tam et al. PNAS. 113:E6639-E6648, 2016; and Schipper at al. J. Control Release. 242:141-147, 2016). Without wishing to be bound by theory, the microneedles and microneedle devices described herein can mimic the natural process of antigen presentation (e.g., viral antigen presentation) by enabling the release, including the controlled- or sustained-release, of a virus-derived antigen, immunogen, and/or vaccine into a subject, for example, into the dermis skin layer of a subject. This sustained release of an antigen can provide numerous advantages over conventional vaccine delivery approaches, including, but not limited to enabling dose-sparing and/or providing increased antigen availability within the lymph nodes, such as during B-cell affinity maturation within germinal centers. The controlled- or sustained-release enabled by the formulations, compositions, articles, devices, preparations, microneedles, and microneedle devices described herein can induce greater immunogenicity, an enhanced immune response which may be characterized by a more potent cellular and/or humoral immune response, and/or broad-spectrum immunity in a subject, as compared to the administration of single-dose or bolus administration of a vaccine, such as a coronavirus vaccine and/or an influenza vaccine (e.g., administered intranasally or subcutaneously).

In some embodiments, the microneedles and microneedle devices described herein comprise an implantable controlled- or sustained-release microneedle tip (e.g., a silk-based microneedle tip) that encapsulates and/or stabilizes a therapeutic agent, such as a vaccine, antigen, and/or an immunogen (e.g., a coronavirus vaccine and/or an influenza vaccine); and a base layer (e.g., a dissolving base layer) that supports the distal microneedle tip. Upon application of the microneedle or microneedle device to a biological barrier of a subject, the base layer can dissolve and the microneedle tips (e.g., silk-based microneedle tips) can be implanted at a predetermined depth (e.g., a max penetration depth of the distal part of tip) within the biological barrier (e.g., the dermis layer of the skin, e.g., at a depth of between about 100 μm and about 800 μm). In some embodiments, the whole tip is embedded within, e.g., the dermis layer of the skin at a depth of between about 100 μm and about 800 μm. The implanted microneedle tip can then slowly release the therapeutic agent over a time period sufficiently long enough to enable immunity to a virus, to prevent infection by a virus, and/or treat a viral infection. In some embodiments, the time period and release kinetics of a therapeutic agent, such as a vaccine, may mimic that of a natural infection by a virus. In some embodiments, the implanted tip can release a therapeutic agent, such as a vaccine, over a time period of at least about 4 days (e.g., about 1, 2, 3, 4, 5, 6, 7, 8, 9, 10, 11, 12, 13, or 14 or more days, e.g., between about 5 days and about 25 days, between about 4 days and about 14 days, between about 10 days and about 20 days, between about 10 days and about 15 days, between about 14 days and about 16 days, between about 14 days and about 15 days, e.g., between about 1-2 weeks, about 1-3 weeks, or about 1-4 weeks, e.g., about one week, about two weeks, about three weeks, about four weeks, about five weeks, or about six weeks or more weeks).

In some embodiments, various properties of a silk fibroin matrix, e.g., of an implantable controlled- or sustained-release microneedle tip, including, for example, crystallinity, beta-sheet content, and molecular weight, can be modulated to tune (e.g., alter and/or modify) the release kinetics (e.g., rate of release) of a vaccine, an antigen, and/or an immunogen (e.g., a coronavirus vaccine and/or influenza vaccine) from the microneedle tip. In some embodiments, the implantable controlled- or sustained-release microneedle tip comprises a beta-sheet content of between about 10% and about 60% (e.g., about 10%, about 20%, about 30%, about 40%, about 50%, about 60%), e.g., as based on a “crystallinity index,” e.g., a “crystallinity index” known in the art.

In another aspect, the present disclosure features microneedles (e.g., silk fibroin-based microneedles) and microneedle devices (e.g., silk-fibroin-based microneedle devices) configured to release a vaccine, antigen, and/or immunogen (e.g., a coronavirus vaccine, an influenza vaccine, or a combination thereof) according to various release kinetics, such as burst release (e.g., immediate or quick dissolution of the microneedle upon application to a biological barrier, such as skin, usually occurring within minutes), and/or sustained release. Examples of sustained release include, but are not limited to, zero order release (e.g., the rate of release is independent of the vaccine, antigen, and/or immunogen concentration in the dosage form, e.g., the release rate is approximately constant over a period of time, e.g., a constant amount of vaccine, antigen, and/or immunogen is eliminated per unit time), first order release (e.g., the rate of release is a function of the amount of the vaccine, antigen, and/or immunogen remaining in the dosage form, e.g., a constant proportion, such as a percentage, of vaccine, antigen, and/or immunogen is eliminated per unit time), and second order release (e.g., where doubling the concentration of vaccine, antigen, and/or immunogen in the dosage for quadruples the release rate). In some embodiments, a portion of a microneedle is configured for a first type of release, e.g., burst release, and another portion of the microneedle is configured for a second type of release, e.g., sustained release. Further, the release (e.g., administration) of a vaccine, antigen, and/or immunogen agent (e.g., a coronavirus vaccine, an influenza vaccine, or a combination thereof) from a microneedle (e.g., a silk fibroin-based microneedle) described herein can be facilitated by the diffusion of the vaccine, antigen, and/or immunogen from the microneedle or a portion thereof; the degradation (e.g., protease mediated degradation) of the microneedle or a portion thereof; and/or the dissolution of the microneedle or a portion thereof. In some embodiments, the microneedles and microneedles devices described herein demonstrate controlled- or sustained-release of a vaccine (e.g., a coronavirus vaccine and/or an influenza vaccine) for at least about 1-3 weeks (e.g., for at least about 1, 2, 3, 4, 5, 6, 7, 8, 9, 10, 11, 12, 13, 14, 15, 16, 17, 18, 19, 20, or 21 days, e.g., between about 5 to about 25 days, e.g., between about 10 to about 15 days), which results in one or more of improved immunogenicity, an enhanced immune response, and/or broad-spectrum immunity. In some embodiments, the microneedles and microneedles devices described herein demonstrate controlled- or sustained-release of a coronavirus vaccine and/or an influenza vaccine for about 5 to about 25 days. In some embodiments, the microneedles and microneedles devices described herein demonstrate controlled- or sustained-release of a coronavirus vaccine and/or an influenza vaccine for about 10 to about 20 days. In some embodiments, the microneedles and microneedles devices described herein demonstrate controlled- or sustained-release of a coronavirus vaccine and/or an influenza vaccine for about 10 to about 15 days.

The microneedles (e.g., silk fibroin-based microneedles) described herein are configured to have sufficient mechanical properties (e.g., strength) and suitable geometry (e.g. tip sharpness, tip included angle, length, inter-needle spacing) to pierce a biological barrier such as skin, e.g., to achieve a local and/or a systemic delivery of a vaccine, antigen, and/or immunogen (e.g., coronavirus vaccine, an influenza vaccine, or a combination thereof) to a subject.

In some embodiments, the microneedles and microneedles devices, e.g., comprising a coronavirus vaccine and/or an influenza vaccine described herein, can be self-administered and are shelf stable. In some embodiments, the microneedles and microneedles devices can provide single-dose protection, e.g., against a coronavirus and/or an influenza virus, and can also protect against strain drift (e.g., by infection mimicry). In some embodiments, the microneedles and microneedles devices described herein can provide seasonal protection, e.g., against a coronavirus and/or influenza virus.

In other embodiments, methods, formulations, compositions, articles, devices, and/or preparations for administering a vaccine, antigen, and/or immunogen (e.g., a coronavirus vaccine and/or an influenza vaccine) that provide improved immunogenicity, an enhanced immune response, and/or a broad-spectrum immunity to a subject are also disclosed. Accordingly, disclosed herein are compositions, preparations, devices (e.g., microneedles and microneedles devices), kits for controlled- and/or sustained release of a vaccine, antigen, and/or immunogen, in a subject, as well as methods of making and using the same.

In some embodiments, the controlled- or sustained-release formulations, compositions, articles, devices, and preparations, comprise at least one vaccine, antigen, and/or immunogen described herein (e.g., a coronavirus vaccine and/or an influenza vaccine). In some embodiments, the formulations, compositions, articles, devices, and preparations for controlled- and/or sustained release described herein release a vaccine, antigen, and/or immunogen over a time period sufficiently long enough to provide immunity to a virus, e.g., a coronavirus and/or influenza virus (e.g., over a time period of at least about 1 to about 25 days (e.g., about 1, 2, 3, 4, 5, 6, 7, 8, 9, 10, 11, 12, 13, 14, or 15 or more days, e.g., between about 4 days and about 25 days, between about 5 days and about 25 days, between about 10 days and about 20 days, between about 10 days and about 15 days, e.g., between about 1-2 weeks, about 1-3 weeks, or about 1-4 weeks, e.g., about one week, about two weeks, about three weeks, about four weeks, about five weeks, or about six weeks or more weeks). Accordingly controlled- or sustained-release formulations, compositions, articles, devices, and preparations, microneedles, microneedle devices, kits, as well as methods of making and using the same are disclosed.

### Definitions

Unless defined otherwise, all technical and scientific terms used herein have the same meaning as commonly understood by one of ordinary skill in the art to which the invention pertains.

The articles “a” and “an” are used herein to refer to one or to more than one (i.e., to at least one) of the grammatical object of the article. By way of example, “an element” means one element or more than one element.

The term “about” when referring to a measurable value such as an amount, a temporal duration, and the like, is meant to encompass variations of ±20%, or in some instances ±10%, or in some instances ±5%, or in some instances ±1%, or in some instances ±0.1% from the specified value, as such variations are appropriate, e.g., to perform the disclosed methods.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein, an “adjuvant” refers to a substance that is able to favor or amplify the cascade of immunological events, ultimately leading to an increased immunological response, e.g., the integrated bodily response to an antigen, including cellular and/or humoral immune responses. Non-limiting examples of adjuvants include: aluminum (e.g., aluminum gels and/or aluminum salts, such as aluminum hydroxide, aluminum phosphate, and aluminum potassium sulfate), lipids (e.g., squalene, monophosphoryl lipid A (MPL)), AS03 (e.g., an adjuvant comprising D,L-alpha-tocopherol (vitamin E), squalene, and polysorbate 80), squalene-based adjuvants (e.g., MF59), cytosine phosphoguanine-based adjuvants (e.g., CpG 1018), adjuvants derived from delta inulin (e.g., Advax adjuvant), and AS04 (e.g., an adjuvant comprising a combination of aluminum hydroxide and MPL).

The term “administration” or “administering” includes routes of introducing a therapeutic agent to a subject to perform their intended function. In certain embodiments, the administration of the therapeutic agent, such as by a microneedle or microneedle device as described herein, may be repeated and the administrations may be separated by at least about 1 day, 2 days, 3 days, 5 days, 10 days, 15 days, 30 days, 45 days, 3 weeks, 4 weeks, 5 weeks, 6 weeks, 12 weeks, 2 months, 75 days, 3 months, or at least 6 months. In other embodiments, the administration of the therapeutic agent, such as by a microneedle or microneedle device as described herein, may be repeated annually. In other embodiments, the administration of the therapeutic agent, such as by a microneedle or microneedle device as described herein, may be repeated as often as necessary to achieve a therapeutic or prophylactic effect. Administration “in combination with” one or more further therapeutic agents includes simultaneous (concurrent) and consecutive administration in any order.

As used herein, the term “antigen” refers to refers to a molecule (e.g., a gene product (e.g., protein or peptide), pathogen fragment, whole pathogen, viral vector, or viral particle) capable of inducing a humoral immune response and/or cellular immune response, e.g., leading to the activation of B and/or T lymphocytes and/or innate immune cells and/or antigen presenting cells. Any macromolecule, including proteins or peptides, can be an antigen. Antigens can also be derived from genomic and/or recombinant DNA. For example, any DNA comprising a nucleotide sequence or a partial nucleotide sequence that encodes a protein capable of eliciting an immune response encodes an “antigen.” In some embodiments, an antigen does not need to be encoded solely by a full length nucleotide sequence of a gene, nor does an antigen need to be encoded by a gene at all. In some embodiments, an antigen can be synthesized or can be derived from a biological sample, e.g., a tissue sample, a tumor sample, a cell, or a fluid with other biological components. In some embodiments, an antigen can be derived from a virus, e.g., an inactivated virus, a viral like particle, or a viral vector. Antigens as used herein may also be mixtures of several individual antigens.

The term “antigen presenting cell” or “APC” refers to an immune system cell such as an accessory cell (e.g., a B-cell, a dendritic cell, and the like) that displays a foreign antigen complexed with major histocompatibility complexes (MHCs) on its surface. T-cells may recognize these complexes using their T-cell receptors (TCRs). APCs process antigens and present them to T-cells.

As used herein, the term “antigenic drift” refers to a mutation in the gene of a virus (e.g., a coronavirus or an influenza virus) that accumulates over time as the virus replicates. These mutations usually produce viruses that are closely related to one another (e.g., located close together on a phylogenetic tree), and referred to herein as “drifted strains.” In some embodiments, viruses that are closely related to each other share similar antigenic properties and an immune system exposed to a first virus and, subsequently, a drifted strain of the first virus will usually recognize the drifted strain and respond to it by mounting an immune response (e.g., a protective immune response), referred to as “cross-protection.” However, in some embodiments these small genetic changes can accumulate over time and result in viruses that are antigenically different (e.g., located further away on a phylogenetic tree), and when this happens, the body's immune system may not recognize those viruses (e.g., those drifted strains).

As used herein, the term “backing” refers to a material that is suitable for bonding to and/or adhering to a component of a microneedle. In some embodiments, a backing material is suitable for bonding to and/or adhering to the base (e.g., dissolvable base) of a microneedle described herein.

As used herein, the term “base” or “dissolvable base” refers to the layer that forms the base of the microneedles (e.g., functions as the support for the distal microneedle tips (e.g., silk fibroin tips) that are loaded with a vaccine, antigen, and/or immunogen (e.g., a coronavirus vaccine, an influenza vaccine, or a combination thereof)), and/or can also serve as a layer connecting adjacent microneedles to form a continuous microneedle array or microneedle patch. In some embodiments, at least 10%, 20%, 30%, 40%, 50%, 60%, 70%, 80%, 90% or more of the base is dissolved after application to a biological barrier, e.g., skin, mucous surface, or buccal cavity.

As used herein, the phrase “broad-spectrum immunity” refers to an immune response, e.g., a humoral and/or cellular response (e.g., immunity or protective immunity), against at least one (e.g., against at least two, at least three, at least four, at least five, against at least eight, or at least against more than eight) strains of a virus (e.g., a virus described herein), wherein the at least one strain (or antigen thereof) is not present in a vaccine administered to a subject, e.g., according to the methods, microneedles, and microneedle devices described herein. In some embodiments, the at least one strain (or antigen thereof) not present in the vaccine, and/or not specifically targeted by the vaccine, is a drifted strain of the virus. In some embodiments, the at least one strain belongs to a different type as the strain(s) present in the vaccine, and or specifically targeted by the vaccine.

As used herein, the term “dose” means the amount of a vaccine (e.g., a coronavirus vaccine and/or an influenza vaccine), antigen, and/or immunogen which is administered (e.g., in a vaccination) to elicit an immune response (e.g., a humoral and/or a cellular immune response) in an organism.

As used herein, a “standard dose” means the amount of antigen in a typical human dose of a vaccine, e.g., as approved for marketing by national or international regulatory authorities (e.g., U.S. FDA, EMEA).

The term “promote” or “enhance” in the context of an immune response refers to an increase in immune response, such as an increase in the ability of immune cells to target and/or kill pathogens and pathogen infected cells, and protective immunity following vaccination, among others. In some embodiments, protective immunity refers to the presence of sufficient immune response (such as antibody titers) to protect against subsequent infection by a pathogen comprising the same antigen. In some embodiments, the pathogen is a virus, such as a coronavirus and/or an influenza virus.

As used herein, a “fractional dose” refers to a dosage comprising a portioned amount of a total dose (e.g., a standard dose) of a vaccine (e.g., a coronavirus vaccine and/or an influenza vaccine), antigen, and/or immunogen which is administered (e.g., in a vaccination) to elicit an immune response (e.g., a humoral and/or a cellular immune response) in an organism. In some embodiments, the amount of the vaccine, antigen, and/or immunogen in the fractional dose is no more than 1/X, wherein X is any number, e.g., wherein X is 0.1, 0.2, 0.3, 0.4, 0.5, 0.6, 0.7, 0.8, 0.9, 1, 2, 3, 4, 5, 6, 7, 8, 9, 10, 15, 20, 30, 40, 50, 60, 70, 80, 90, or 100 or more, of the total dose (e.g., a standard dose) of the vaccine.

As used herein, the term “gelatin” refers to a water-soluble protein derived from collagen. In some embodiments, the term “gelatin” refers to a sterile nonpyrogenic protein preparation (e.g., fractions) produced by partial acid hydrolysis (type A gelatin) or by partial alkaline hydrolysis (type B gelatin) of animal collagen, most commonly derived from cattle, pig, and fish sources. Gelatin can be obtained in varying molecular weight ranges. Recombinant sources of gelatin may also be used.

As used herein, the term “immunity” or “protective immunity” refers to an immune response, e.g., a humoral and/or cellular response, elicited by a vaccine or immunization schedule (e.g., vaccination regimen) that when administered to a subject in need thereof (e.g., a subject described herein), that prevents, retards the development of, and/or reduces the severity of a viral infection that is caused by a virus described herein. In some embodiments, immunity or protective immunity diminishes or altogether eliminates the symptoms of the viral infection. In some embodiments, immunity or protective immunity is characterized by the presence of one or more of: circulating antibodies (e.g., humoral immunity), the presence of sensitized T lymphocytes (e.g., cellular immunity), the presence of secretory IgA on mucosal surfaces (e.g., mucosal immunity), or a combination thereof.

As used herein, the term “immunogen” refers to any substance (e.g., an antigen, combination of antigens, pathogen fragment, whole pathogen) capable of eliciting an immune response in an organism. An “immunogen” is capable of inducing an immunological response against itself after administration to a mammalian subject. The term “immunological” as used herein with respect to an immunological response, refers to the development of a humoral (antibody mediated) and/or a cellular (mediated by antigen-specific T cells or their secretion products) response directed against an immunogen in a recipient subject. Such a response can be an active response induced by administration of an immunogen or immunogenic peptide to a subject or a passive response induced by administration of antibody or primed T cells that are directed towards the immunogen. In some embodiments, an immunogen is a coronavirus antigen. In some embodiments, an immunogen is a coronavirus. In some embodiments, an immunogen is an influenza virus. In some embodiments, an immunogen is a viral vaccine (e.g., a monovalent (also called univalent) or a multivalent (also called polyvalent) vaccine, such as for coronavirus and/or influenza). In some embodiments, the vaccine (e.g., coronavirus vaccine and/or influenza vaccine) may be monovalent, bivalent, trivalent, quadrivalent (also called tetravalent), or pentavalent. In some embodiments, the immunogen is a replicating or non-replicating vaccine vector (e.g., comprises an adenovirus vector, an adeno-associated virus vector, an alpha virus vector, a herpesvirus vector, a measles virus vector, a poxvirus vector, or a vesicular stomatitis virus vector).

As used herein, the term “immunogenicity” refers to the ability of a substance, such as an antigen or epitope, to provoke humoral and/or cell-mediated immunological response in a subject. A skilled artisan can readily measure immunogenicity of a substance. The presence of a cell-mediated immunological response can be determined by any art-recognized methods, e.g., proliferation assays (CD4+ T cells), CTL (cytotoxic T lymphocyte) assays, or immunohistochemistry with tissue section of a subject to determine the presence of activated cells such as monocytes and macrophages after the administration of an immunogen. One of skill in the art can readily determine the presence of humoral-mediated immunological response in a subject by any well-established methods. For example, the level of antibodies produced in a biological sample such as blood can be measured by western blot, ELISA or other methods known for antibody detection.

As used interchangeably herein, the terms “sustained-release tip,” “implantable sustained-release tip,” “implantable microneedle tip,” or “releasable tip” refers to the distal end, e.g., tip, of a microneedle capable of piercing a biological barrier, e.g., the skin, mucous surface, or buccal cavity, of a subject and being deposited within the biological barrier, a skin layer (e.g., the dermis). In embodiments, the tip comprises a silk fibroin protein in an amount sufficient to sustain the release of a vaccine, e.g., a coronavirus vaccine (e.g., a SARS-CoV-2 vaccine) and/or an influenza vaccine for a prolonged period of time, e.g., for at least about 1 day (e.g., about 1, 2, 3, 4, 5, 6, 7, 8, 9, 10, 11, 12, 13, or 14 or more days, e.g., between about 4 days and about 30 days, 5 days about 25 days, between about 10 days and about 20 days, between about 10 days and about 15 days, between about 4 days and about 14 days, between about 14 days and about 15 days, e.g., between about 1-2 weeks, about 1-3 weeks, or about 1-4 weeks, e.g., about 2-12 months). In some embodiments, the implantable sustained-release tip comprises a coronavirus vaccine, antigen, and/or immunogen. In some embodiments, the implantable sustained-release tip comprises an influenza vaccine, antigen, and/or immunogen.

As used herein, the term “microneedle” refers to a structure having at least two, more typically, three components, e.g., layers, for transport or delivery of a vaccine, an antigen, and/or an immunogen, across a biological barrier, such as the skin, tissue, or cell membrane. In some embodiments, a microneedle comprises a base (e.g., a dissolvable base as described herein), a tip (e.g., an implantable tip as described herein), and optionally, a backing material. In embodiments, a microneedle has dimension of between about 350 μm to about 1500 μm in height (e.g., between about 350 μm to about 1500 μm, e.g., about 350 μm, about 400 μm, about 450 μm, about 500 μm, about 550 μm, about 600 μm, about 650 μm, about 700 μm, about 750 μm, about 800 μm, about 850 μm, about 900 μm, about 950 μm, about 1000 μm, about 1050 μm, about 1100 μm, about 1150 μm, about 1200 μm, about 1250 μm, about 1300 μm, about 1350 μm, about 1400 μm, about 1450 μm, about 1500 μm)). In some embodiments, the microneedle is fabricated to have any dimension and/or geometry to enable the deployment of a microneedle tip (e.g., a silk fibroin tip), e.g., an implantable sustained-release tip, at a depth between about 100 μm and about 900 μm (e.g., at a depth of about 800 μm) into the dermis layer of the skin for release, e.g., controlled- or sustained-release of a vaccine (e.g., a coronavirus vaccine and/or an influenza vaccine).

As used herein, the term “microneedle patch” and “microneedle array” refers to a device comprising a plurality of microneedles, e.g., silk fibroin-based microneedles, e.g., arranged in a random or predefined pattern, such as an array.

As used herein, the term “polyethylene glycol (PEG)” refers to an oligomer or polymer of ethylene oxide. PEG is also known as polyethylene oxide (PEO) or polyoxyethylene (POE). The structure of PEG is commonly expressed as H—(O—CH₂—CH₂)ₙ—OH.

As used herein, the term “silk fibroin” includes silkworm fibroin and insect or spider silk protein. Any type of silk fibroin can be used according to various aspects described herein. Silk fibroin produced by silkworms, such as Bombyx mori, is the most common and represents an earth-friendly, renewable resource. For instance, silk fibroin used in a microneedle (e.g., a silk fibroin tip, e.g., an implantable controlled- or sustained-release tip of a microneedle) may be obtained by removing sericin from the cocoons of B. mori. In some embodiments, the silk fibroin is a regenerated silk fibroin, e.g., a silk fibroin obtained after extraction of sericin from the cocoons of B. mori, and an additional processing e.g. via a boiling step. Organic silkworm cocoons are also commercially available. There are many different silks, however, including spider silk (e.g., obtained from Nephila clavipes), transgenic silks, recombinant and/or genetically engineered silks, such as silks from bacteria, yeast, mammalian cells, transgenic animals, or transgenic plants (see, e.g., WO 97/08315; U.S. Pat. No. 5,245,012), and variants thereof, that can be used.

As used herein, the term “release” and “controlled- or sustained-release” refers to the release of a vaccine, an antigen, and/or an immunogen (e.g., from a microneedle, microneedle device, formulation, composition, article, device, and preparation described herein, e.g., from a silk fibroin-based microneedle tip as described herein), such as a coronavirus vaccine, an influenza vaccine, or a combination thereof, over a period of time, e.g., for at least about 1 to about 28 days (e.g., about 1, 2, 3, 4, 5, 6, 7, 8, 9, 10, 11, 12, 13, 14, 15, 16, 17, 18, 19, 20, 21, 22, 23, 24, 25, 26, 27, or 28 or more days, e.g., between about 4 days and about 25 days, between about 10 and about 20 days, between about 10 and about 15 days, between about 12 and about 16 days, e.g., between about 1-2 weeks, about 1-3 weeks, or about 1-4 weeks, e.g., between about 1 month to about 3 months). In some embodiments, the controlled- or sustained-release of a vaccine, such as a coronavirus vaccine and/or an influenza vaccine, over a time period of about 1 to about 14 days, e.g., about 1, 2, 3, 4, 5, 6, 7, 8, 9, 10, 11, 12, 13, or 14 days, by a microneedle, microneedle device, formulation, composition, article, device, or preparation as described herein can result, e.g., in broad-spectrum immunity in a subject. In some embodiments, the vaccine formulations and preparations comprising silk fibroin have controlled- or sustained-release properties (e.g., are formulated and/or configured to release a vaccine, e.g., into the skin of the subject, over a period of, or at least 1, 5, 10, 15, 30, 45 minutes; a period of, or at least, 1, 2, 3, 4, 5, 10, 24 hours; a period of, or at least, 1, 2, 3, 4, 5, 6, 7, 8, 9, 10, 11, 12, 13, 14 days; a period of, or at least, 1, 2, 3, 4, 5, 6, 7, 8 weeks; a period of, or at least 1, 2, 3, 4, 5, 6, 7, 8, 9, 10, 11 months; a period of, or at least, 1, 2, 3, 4, 5 years, or longer.

As used herein, a “subject” refers to a human or animal. Usually the animal is a vertebrate such as a primate, rodent, domestic animal, or game animal. Primates include chimpanzees, cynomolgus monkeys, spider monkeys, and macaques (e.g., Rhesus). Rodents include mice, rats, woodchucks, ferrets, rabbits and hamsters. Domestic and game animals include cows, horses, pigs, deer, bison, buffalo, feline species (e.g., domestic cat), canine species (e.g., dog, fox, wolf), avian species (e.g., chicken, emu, ostrich), and fish (e.g., trout, catfish and salmon). In certain embodiments of the aspects described herein, the subject is a mammal (e.g., a primate, e.g., a human). A subject can be male or female. In certain embodiments, the subject is a mammal. The mammal can be a human, non-human primate, mouse, rat, dog, cat, horse, or cow, but are not limited to these examples. In addition, the methods and formulations described herein can be used to treat domesticated animals and/or pets. In some embodiments, the term “subject” is intended to include living organisms in which an immune response can be elicited (for example, mammals, for example, human).

In a particular embodiment, the subject is a human. A subject may be of any age. In an embodiment, the subject is an elderly human subject, e.g., 65 years of age or older. In an embodiment, a subject is a human subject who is not an elderly, e.g., less than 65 years of age. In an embodiment, a subject is a human pediatric subject, e.g., 18 years of age or less. In an embodiment, a subject is an adult subject, e.g., older than 18 years of age.

As used herein, the term “therapeutic agent” and “active agent” are art-recognized terms and refers to any chemical moiety that is a biologically, physiologically, or pharmacologically active substance that acts locally or systemically in a subject. Various forms of a therapeutic agent may be used which are capable of being released from the microneedles described herein into adjacent tissues or fluids upon administration to a subject. Examples of therapeutic agents, also referred to as “drugs”, are described in well-known literature references such as the Merck Index, the Physicians Desk Reference, and The Pharmacological Basis of Therapeutics, and they include, without limitation, medicaments; vitamins; mineral supplements; substances used for the treatment, prevention, diagnosis, cure or mitigation of a disease or illness, such as a viral infection; substances which affect the structure or function of the body; or pro-drugs, which become biologically active or more active after they have been placed in a physiological environment.

In certain embodiments, a therapeutic agent comprises, without limitation, a vaccine, an antigen, and/or an immunogen. In certain embodiments, a therapeutic agent comprises a coronavirus vaccine, antigen, and/or immunogen. In certain embodiments, a therapeutic agent comprises an influenza vaccine, antigen, and/or immunogen.

In certain embodiments, a therapeutic agent comprises, without limitation, an amino acid molecule, such as a peptide and/or a protein. In certain embodiments, a therapeutic agent comprises a recombinant protein vaccine.

In certain embodiments, a therapeutic agent comprises, without limitation, a nucleic acid molecule, such as a deoxyribonucleic acid (DNA) molecule and/or a ribonucleic acid (RNA) molecule. In particular embodiments, a therapeutic agent comprises an mRNA. In some embodiments, a therapeutic agent comprises a nucleic acid based vaccine, such as a DNA-based vaccine and/or a RNA-based vaccine. In some embodiments, a therapeutic agent comprises an mRNA-based vaccine.

As used herein, the term “vaccine” refers to any composition that will elicit a protective immune response in a subject that has been exposed to the composition. An immune response may include induction of antibodies and/or induction of a T-cell response. Usually, an “immune response” includes but is not limited to one or more of the following effects: the production or activation of antibodies, B cells, helper T cells, suppressor T cells, and/or cytotoxic T cells, directed specifically to an antigen or antigens included in or derived from the composition or vaccine of interest. Preferably, the subject will display either a therapeutic or a protective immunological (memory) response such that resistance to new infection will be enhanced and/or the clinical severity of the disease reduced. Such protection will be demonstrated by either a reduction in number or severity of, or lack of one or more of the clinical signs associated with the infection of the pathogen, in the delay of onset of viremia, in a reduced viral persistence, in a reduction of the overall viral load and/or in a reduction of viral excretion. In some embodiments, a “vaccine” refers to any preparation of an antigen or an immunogen (including subunit antigens, toxoid antigens, conjugate antigens, or other types of antigenic molecules, or nucleic acid molecules encoding the same) or a killed or live attenuated microorganism that, when introduced into a subject's body, affects the immune response to the specific antigen or microorganism by causing activation of the immune system against the specific antigen or microorganism (e.g., inducing antibody formation, T-cell responses, and/or B-cell responses). Generally, vaccines against microorganisms are directed toward at least part of a virus, bacteria, parasite, mycoplasma, or other infectious agent.

The term “therapeutically effective amount” refers to an amount of the composition as defined herein that is effective for preventing, ameliorating and/or treating a condition resulting from a disease as described herein, such as a viral infection.

The term “treatment” refers to therapeutic treatment as well as prophylactic or preventative measures to cure or halt or at least retard disease progress. Those in need of treatment include those already inflicted with a condition resulting from infection with a virus as described herein as well as those in which infection with a virus is to be prevented. Subjects partially or totally recovered form infection with a virus as described herein might also be in need of treatment. Prevention encompasses inhibiting or reducing the spread of a virus or inhibiting or reducing the onset, development or progression of one or more of the symptoms associated with infection with a virus described herein.

The term “viral titer” or “viral load” interchangeably refers to a measure of the severity of an active viral infection, and can be determined by methods known to the person skilled in the art. The determination can be based on the detection of viral proteins such as by antibody binding to the viral proteins and further detection or, alternatively, by detection of viral DNA and/or viral RNA by amplification methods such as PCR and RT-PCR. Monitoring of virion associated viral RNA in plasma by nucleic acid amplification methods is a widely used parameter to assess the status and progression of viral disease, and to evaluate the effectiveness of prophylactic and therapeutic interventions. In certain embodiments, the virus load or virus titer can be calculated by estimating the live amount of virus in a sample of body fluid such as a number of RNA copies per milliliter of a blood sample.

As used herein, the term “viruses” refers to an infectious agent composed of a nucleic acid encapsidated in a protein. Such infectious agents are incapable of autonomous replication (i.e., replication requires the use of the host cell's machinery). Viral genomes can be single-stranded (ss) or double-stranded (ds), RNA or DNA, and can or cannot use reverse transcriptase (RT). Additionally, ssRNA viruses can be either sense (+) or antisense (−). Exemplary viruses include, but are not limited to, dsDNA viruses (e.g., Adenoviruses, Herpesviruses, Poxviruses), ssDNA viruses (e.g., Parvoviruses), dsRNA viruses (e.g., Reo viruses), (+)ssRNA viruses (e.g., Picornaviruses, Toga viruses, coronaviruses), (−)ssRNA viruses (e.g., Orthomyxoviruses, Rhabdoviruses), ssRNA-RT viruses, i.e., (+)sense RNA with DNA intermediate in life-cycle (e.g., Retroviruses), and dsDNA-RT viruses (e.g., Hepadnaviruses). In some embodiments, viruses can also include wild-type (natural) viruses, killed viruses, live attenuated viruses, modified viruses, recombinant viruses or any combinations thereof. Exemplary retroviruses include human immunodeficiency virus (HIV). Other examples of viruses include, but are not limited to, enveloped viruses, respiratory syncytial viruses, non-enveloped viruses (e.g., human papillomavirus (HPV)), bacteriophages, recombinant viruses, and viral vectors. The term “bacteriophages” as used herein refers to viruses that infect bacteria.

As used herein, the term “coronavirus” refers to a positive-sense ssRNA virus within the Coronaviridae family. A coronavirus may be an alphacoronavirus, a betacoronavirus, a gammacoronavirus, or a deltacoronavirus. A coronavirus can be a live wild-type virus, a live attenuated virus, an inactivated virus (e.g., a UV-inactivated virus), a chimeric virus, or a recombinant virus. Coronaviruses are known to infect humans and other animals (e.g., birds and mammals). Examples of coronaviruses include severe acute respiratory syndrome coronavirus (SARS-CoV), severe acute respiratory syndrome virus 2 (SARS-CoV-2), Middle East respiratory syndrome coronavirus (MERS-CoV), human coronavirus 229E (HCoV-229E), human coronavirus NL63 (HCoV-NL63), human coronavirus OC43 (HCoV-OC43), and human coronavirus HKU1 (HCoV-HKU1).

As used herein, the term “influenza virus” refers to a negative-sense ssRNA virus within the Orthomyxoviridae family. An influenza virus can be a live wild-type virus, a live attenuated virus, an inactivated virus, a chimeric virus, or a recombinant virus. Examples of influenza viruses include influenza A, influenza B, influenza C, and influenza D.

Ranges: throughout this disclosure, various embodiments can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98%, or 99% identity, and includes subranges such as 96-99%, 96-98%, 96-97%, 97-99%, 97-98%, and 98-99% identity. This applies regardless of the breadth of the range.

Various embodiments of the compositions and methods herein are described in further detail below. Additional definitions are set out throughout the specification.

### Microneedle Devices

Provided herein are various microneedles and microneedle devices comprising silk fibroin protein that are configured to encapsulate and release an effective amount of a therapeutic agent, such as a vaccine, to a subject. In certain embodiments, provided herein are microneedles, e.g., silk fibroin-based microneedles, pluralities of microneedles, and microneedle devices (e.g., microneedle patches), e.g., silk fibroin-based microneedle devices, configured to incorporate, and subsequently release (e.g., administer), an effective amount of a vaccine, an antigen, and/or an immunogen (e.g., a coronavirus vaccine (e.g., a SARS-CoV-2 vaccine), an influenza vaccine, or a combination thereof) into a subject (e.g., into and/or across a biological barrier, such as the skin, of a subject). Use of the microneedles, pluralities of microneedles, and microneedle devices can result in an immunity (e.g., a prolonged, broad spectrum immunity) to a virus and/or a virus-associated antigen in a subject, such as a coronavirus or coronavirus-associated antigen, and/or an influenza virus or influenza-virus associated antigen.

The microneedles described herein can be in any shape and/or geometry suitable for use in piercing a biological barrier, e.g., a layer of the skin, e.g., a shape or geometry described herein. The microneedles may comprise two or more layers, such as a backing material, a base layer (e.g., a dissolvable base layer), and a microneedle tip (e.g., an implantable microneedle tip) which are described in more detail herein.

The microneedles described herein can include silk fibroin protein in any suitable amount. For example, the microneedles or a portion thereof, such as a microneedle tip, may comprise between about 0.1% and about 20% v/v silk fibroin protein, or between about 0.1% and about 10% v/v silk fibroin protein, optionally, wherein the percentage (%) is based on the silk fibroin solution (“print solution”) used during fabrication. For example, microneedles or a portion thereof, such as a microneedle tip, may comprise at least about 0.1%, about 0.25%, about 0.5%, about 0.75%, about 1%, about 1.25%, about 1.5%, about 1.75%, about 2%, about 2.25%, about 2.5%, about 2.75%, about 3%, about 3.25%, about 3.5%, about 3.75%, about 4%, about 4.25%, about 4.5%, about 4.75%, about 5%, about 5.25%, about 5.5%, about 5.75%, about 6%, about 6.25%, about 6.5%, about 6.75%, about 7%, about 7.25%, about 7.5%, about 7.75%, about 8%, about 8.25%, about 8.5%, about 8.75%, about 9%, about 9.25%, about 9.5%, about 9.75%, about 10%, about 10.5%, about 11%, about 11.5%, about 12%, about 12.5%, about 13%, about 13.5%, about 14%, about 14.5%, about 15%, about 15.5%, about 16%, about 16.5%, about 17%, about 17.5%, about 18%, about 18.5%, about 19%, about 19.5%, or about 20% v/v silk fibroin protein, optionally, wherein the percentage (%) is based on the silk fibroin solution (“print solution”) used during fabrication.

In some embodiments, the silk fibroin protein is from a silk worm (Bombyx Mori). In some embodiments, the silk fibroin protein comprises spider silk. In some embodiments, the silk fibroin protein does not comprises spider silk. In some embodiments, the silk fibroin protein is in the form of a fiber or a particle. In some embodiments, the silk fibroin protein does not comprise a fiber or a particle.

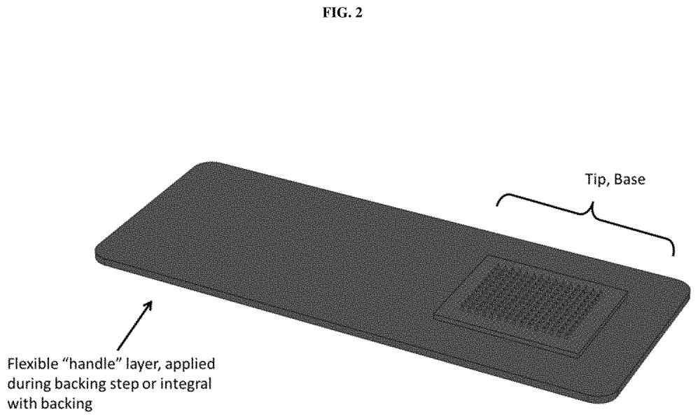

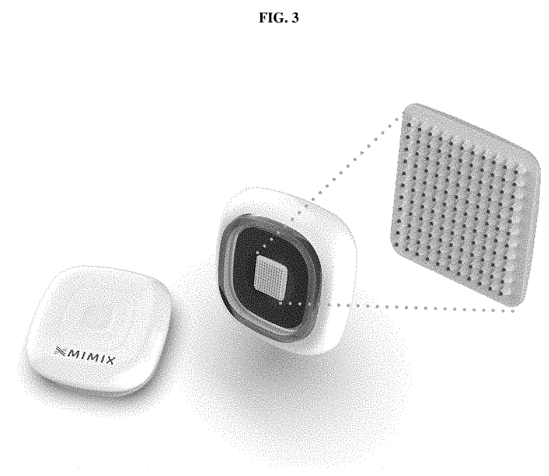

In some embodiments, a plurality of microneedles can be arranged in a random or predefined pattern to form a microneedle array and/or patch, as described herein (see, e.g., FIG. 3). The patch may comprise a carrier, backing, or “handle” layer adhered to the back of the base (see, e.g., FIG. 2). This layer can provide structural support and an area by which the patch can be handled and manipulated without disturbing the needle array.

The microneedle device (e.g., microneedle patch) may comprise at least about 100 or more microneedles, e.g., between about 100 to about 500 microneedles, e.g., between about 100 to about 400 microneedles, between about 100 to about 300 microneedles, or between about 100 to about 200 microneedles, e.g., about 121 microneedles. The microneedles may be arranged in a grid, e.g., a square grid. In some embodiments, the microneedle device comprises about 121 needles, e.g., in an 11×11 square grid. In some embodiments, the microneedle device comprises about 121 needles in an 11×11 square grid with approximately 0.75 mm pitch.

In some embodiments, individual microneedles needles are cones approximately 0.65 mm long with base diameter approximately 0.35 mm and included angle of approximately 30°. In some embodiments, the tip of the needle is sufficiently sharp to penetrate a biological barrier, e.g., the skin. In some embodiments, the radius of curvature of the microneedle tip is no more than 0.01 mm.

In one aspect, the present disclosure features solid pyramidal microneedle arrays fabricated with silk fibroin protein tips encapsulating a therapeutic agent, such as a vaccine, supported on a dissolving polymer base. Upon brief skin application, vaccine-loaded silk tips can be implanted into the epidermis/upper dermis of a subject where they release vaccine over a time period determined by various tunable properties of the silk matrix, including, for example, the crystallinity of the silk matrix.

In another aspect, the present disclosure features solid pyramidal microneedle arrays fabricated with silk fibroin protein tips encapsulating a therapeutic agent, such as an mRNA, supported on a dissolving polymer base. Upon brief skin application, mRNA-loaded silk tips can be implanted into the epidermis/upper dermis of a subject where they release mRNA over a time period determined by various tunable properties of the silk matrix, including, for example, the crystallinity of the silk matrix.

### Silk Fibroin-Based Microneedles

In some embodiments, the present disclosure provides silk fibroin-based microneedles and microneedle devices (e.g., microneedle patches) for the administration of an effective amount of a therapeutic agent, such as a vaccine, to a subject in need thereof.

In some embodiments, the present disclosure provides silk fibroin-based microneedles and microneedle devices (e.g., microneedle patches) for the transport and release of an effective amount of a vaccine, an antigen, and/or an immunogen, such as a coronavirus vaccine, an influenza vaccine, or a combination thereof, into and/or across a biological barrier (e.g., skin, mucosa, tissue, such as organ tissue and muscle tissue, buccal cavity, oral cavity, or a cell membrane).

Accordingly, the silk fibroin-based microneedles and microneedle devices disclosed herein can be configured to have various mechanical properties (e.g., strength), designs and geometries (e.g., needle shape and sharpness), and release kinetics (e.g., sustained release) to enable the administration of an effective amount of a vaccine, an antigen, and/or an immunogen, such as a coronavirus vaccine, an influenza vaccine, or a combination thereof, to a subject, e.g., to protect against an infection (e.g., an infection by a coronavirus and/or an influenza virus) in the subject.

In another aspect, the present disclosure features microneedles (e.g., silk-fibroin based microneedles) that can stabilize a vaccine, antigen, and/or immunogen (e.g., a coronavirus vaccine and/or an influenza vaccine) disposed in the microneedle, e.g., due in part to the thermostabilization properties of the microneedle composition (e.g., the thermostabilization properties of the silk fibroin composition). In some embodiments, a coronavirus vaccine (e.g., SARS-CoV-2 vaccine, SARS-CoV vaccine, and/or MERS-CoV vaccine) and/or an influenza vaccine is stabilized by a microneedle or microneedle device described herein.

In another aspect, the present disclosure features microneedles that can stabilize an mRNA disposed in the microneedle.

In some embodiments, the coronavirus vaccine and/or influenza vaccine retains at least 50% (e.g., about 50%, 60%, 70%, 80%, 90%, 92%, 94%, 96%, 98%, 99%, 99.5%, or more) of its original bioactivity (e.g., immunogenicity) after storage for a period of 2 or more weeks, e.g., at room temperature (e.g., about 25° C.). In some embodiments, the coronavirus vaccine and/or influenza vaccine retains at least 50% (e.g., about 50%, 60%, 70%, 80%, 90%, 92%, 94%, 96%, 98%, 99%, 99.5%, or more) of its original bioactivity (e.g., immunogenicity) after storage for a period of 8 or more weeks, e.g., at room temperature (e.g., about 25° C.).

In some embodiments, the coronavirus vaccine and/or the influenza vaccine retains at least 70%, 80%, or 90% of its original bioactivity (e.g., immunogenicity) after storage at about 25° C., for at least about 2 weeks (e.g., for about 2 weeks, about 3 weeks, about 4 weeks, about 5 weeks, about 6 weeks, about 7 weeks, about 8 weeks, about 9 weeks, about 10 weeks, about 11 weeks, or about 12 weeks). In some embodiments, the coronavirus vaccine and/or the influenza vaccine retains at least 70%, 80%, or 90% of its original bioactivity (e.g., immunogenicity) after storage at about 25° C., for at least about 8 weeks (e.g., for about 8 weeks, about 9 weeks, about 10 weeks, about 11 weeks, or about 12 weeks).

In some embodiments, the coronavirus vaccine and/or the influenza vaccine retains at least 60%, 70%, or 80% of its original bioactivity (e.g., immunogenicity) after storage at about 37° C., for at least about 2 weeks (for about 2 weeks, about 3 weeks, about 4 weeks, about 5 weeks, about 6 weeks, about 7 weeks, about 8 weeks, about 9 weeks, about 10 weeks, about 11 weeks, or about 12 weeks).

In some embodiments, the coronavirus vaccine and/or the influenza vaccine retains at least 50%, 60%, or 70% of its original bioactivity (e.g., immunogenicity) after storage at about 45° C., for at least about 2 weeks (for about 2 weeks, about 3 weeks, about 4 weeks, about 5 weeks, about 6 weeks, about 7 weeks, about 8 weeks, about 9 weeks, about 10 weeks, about 11 weeks, or about 12 weeks).

### Mechanical Properties

Microneedles, including the silk fibroin-based microneedles disclosed herein, can be designed for insertion into the skin without breaking. In some embodiments, microneedle insertion is achieved by using needles with sharp tips and with sufficient length to overcome the deflection of a biological barrier's (e.g., the skin's) surface that occurs before insertion. In some embodiments, microneedle integrity during insertion can be achieved by minimizing the required insertion force, e.g., by using sharp-tipped needles, by maximizing the mechanical strength, and/or by optimizing the needle diameter (See, e.g., Park et al. J. Korean Phys. Soc. 56(4): 1223-1227, 2010). Accordingly, in some embodiments, the mechanical properties of the silk fibroin-based microneedles are optimized (e.g., by adjusting the concentration of various formulation components, including silk fibroin crystallinity, disclosed herein) to avoid sudden failure of a microneedle by buckling, and to enable successful penetration and insertion of the microneedle into the biological barrier (e.g., skin). In some embodiments, the microneedles disclosed herein are configured to have geometries below a 4:1 aspect ratio of length-to-equivalent diameter and/or a to have mechanical strength characterized by Young's modulus greater than 500 MPa and failure stress greater than 10 MPa. In some embodiment, the microneedles have a 15 degree included angle and/or about a 4:1 aspect ratio. In some embodiments, the base formula has a Flex modulus of about 1000 to about 1500 MPa and a failure stress of about 15 to about 30 MPa.

### Microneedle Designs

In some embodiments, the present disclosure provides microneedles, such as silk fibroin-based microneedles, and devices comprising the same, that have various design configurations. The microneedles (e.g., silk fibroin-based microneedles) disclosed herein can be in any shape and/or geometry suitable for use in piercing a biological barrier (e.g., skin) to enable release, e.g., sustained-release, of a vaccine, an antigen, and/or an immunogen (e.g., a coronavirus vaccine and/or an influenza vaccine) within a subject. Non-limiting examples of the shape and/or geometry of the microneedles include: a cylindrical shape, a wedge-shape, a cone-shape, a pyramid-shape, a diamond-shape, and/or an irregular-shape, or any combinations thereof. In some embodiments, the shape and/or geometry of the microneedles does not comprise a diamond shape. In some embodiments, the shape and/or geometry of the microneedles comprises a pyramid-shape.

The microneedles disclosed herein can be fabricated in any suitable format. Non-limiting examples of the format of the microneedles include: solid microneedles, hollow microneedles, coated microneedles, dissolving microneedles, implantable microneedles, hydrogel microneedles, or any combinations thereof. In some embodiments, the microneedles (e.g., silk fibroin-based microneedles) comprise a solid supporting shaft. In some embodiments, the microneedles (e.g., silk fibroin-based microneedles) do not comprise a solid supporting shaft.

In some embodiments, the microneedles (e.g., silk fibroin-based microneedles) are comprised of dissolving and/or degradable microneedles. In some embodiments, a dissolvable and/or degradable (e.g., resorbable) microneedle of the present disclosure encapsulates the vaccine, antigen, and/or immunogen (e.g., coronavirus vaccine and/or an influenza vaccine) in a formulation, such as a silk fibroin-based formulation, which dissolves and/or degrades once inside the subject (e.g., skin). In some embodiments, the release of a vaccine, an antigen, and/or an immunogen (e.g., a coronavirus vaccine and/or an influenza vaccine) from a degradable microneedle is by protease mediated degradation. In some embodiments, only a portion (e.g., tip, e.g., silk fibroin tip) of the microneedle is configured to be dissolvable and/or degradable. In some embodiments, substantially all of the microneedle is dissolvable and/or degradable. In some embodiments, the release of a vaccine, an antigen, and/or an immunogen (e.g., a coronavirus vaccine and/or an influenza vaccine) from a dissolvable and/or degradable (e.g., resorbable) microneedle is by diffusion-controlled release through the material of the microneedle.

In some embodiments, the microneedles (e.g., silk fibroin-based microneedles) are solid microneedles. In some embodiments, a solid microneedle of the present disclosure is designed as a two-part system. In some embodiments, a microneedle device comprising silk fibroin-based solid microneedles is first applied to the skin to create microscopic wells just deep enough to penetrate the outermost layer of a biological barrier (e.g., skin), and then the vaccine, antigen, and/or immunogen (e.g., coronavirus vaccine and/or influenza vaccine) is applied via a transdermal patch. In some embodiments, the release of a vaccine, an antigen, and/or an immunogen (e.g., a coronavirus vaccine and/or an influenza vaccine) from a solid microneedle is by diffusion through the material of the microneedle and/or degradation of the material of the microneedle, e.g., protease mediated degradation. In some embodiments where the solid microneedles degrade, the solid microneedle are typically referred to as dissolving or resorbable microneedles.

In some embodiments, the microneedles (e.g., silk fibroin-based microneedles) are hollow microneedles. In some embodiments, a hollow microneedle of the present disclosure comprises a reservoir that delivers the vaccine, antigen, and/or immunogen (e.g., a coronavirus vaccine and/or an influenza vaccine) directly into the site of application (e.g., a biological barrier, e.g., skin).

In some embodiments, the microneedles (e.g., silk fibroin-based microneedles) are coated microneedles, e.g., coated with a coronavirus antigen and/or an influenza antigen, or a vaccine preparation thereof. In some embodiments, the vaccine, antigen, and/or immunogen (e.g., coronavirus vaccine and/or influenza vaccine) is applied directly to a portion (e.g., a surface) of a microneedle. In some embodiments, the coated microneedles are also coated with a surfactant (e.g., an octyl phenol ethoxylate (e.g., Triton-X), polysorbate, poloxamers, such as P188, and/or a polyethoxylated alcohol) and/or a thickening agent, e.g., to assure that the vaccine, an antigen, and/or an immunogen is delivered properly.

In some embodiments, the microneedles described herein may include a pore or a plurality of pores. In other embodiments, the microneedles described herein do not include a pore or a plurality of pores.

In some embodiments, the microneedles described herein may include a reservoir or a plurality of reservoirs. In other embodiments, the microneedles described herein do not include a reservoir or a plurality of reservoirs.

In some embodiments, a microneedle (e.g., silk fibroin-based microneedle) of the present disclosure can comprise the following layers: (1) a backing material (optional); (2) a base (e.g., a dissolvable base); and (3) a tip (e.g., a silk fibroin tip). For example, the microneedles may include a backing material (optional) applied to a dissolvable base layer that supports a distal silk fibroin tip comprising a silk fibroin and a therapeutic agent, such as a vaccine, an antigen, and/or an immunogen (e.g., a coronavirus vaccine and/or an influenza vaccine).

### Backing

The microneedles and microneedle devices described herein may feature a backing layer. The backing may be applied to the base (e.g., dissolvable base), and can facilitate handling, demolding, and/or application of the microneedles or microneedle devices.

Exemplary backing materials that can be used in the fabrication of a microneedle of the present disclosure include, but are not limited to, a solid support, e.g., a paper-based material, a plastic material, a polymeric material, or a polyester-based material (e.g., a Whatman 903 paper, a polymeric tape, a plastic tape, an adhesive-backed tape (e.g., adhesive-backed polyester tape), or other suitable tape). In some embodiments, the backing comprises Whatman 903 paper. In some embodiments, the backing comprises a polyester tape. In some embodiments, the polyester tape comprises an adhesive-backed polyester tape. In some embodiments, the backing material may be coated (e.g., at least on one side) with an adhesive suitable for bonding to and/or adhering to the dissolvable base of a microneedle described herein. In some embodiments, the backing may extend beyond the microneedle array. In some embodiments, the adhesive coated on the backing may be suitable for adhering to the subject's skin to hold the array in place.

The backing materials used in the microneedles of the present disclosure may have various properties, including, but not limited to, the ability to bond and/or adhere to the base (e.g., dissolvable base) layer to permit demolding. In some embodiments, a backing material maintains patch integrity, e.g., if the dissolving base layer has cracks or discontinuities. The backing material may be sufficiently flexible so as to conform, for example, to a non-flat surface, such as a skin surface. In particular, the backing can be flexible enough during wear time, such as after the patch is applied (e.g., pressed into) the skin. The backing may comprise and/or consist of a non-dissolving material, such that the backing maintains its integrity after patch application to a skin surface and during patch removal from a skin surface.

In some embodiments the backing comprises an adhesive. In some embodiments, the backing comprises an adhesive that comprises a solid support. In some embodiments, the backing comprises an adhesive and a porous support matrix. The backing may comprise an adhesive that can be treated, e.g., after subjecting the backing to conditions sufficient to treat the adhesive, e.g., by temperature, oxidation, and/or UV irradiation. In some embodiments, the backing comprises an adhesive that provides a connection to a solid support (e.g., a porous support matrix).

The backing may have any dimension suitable for application to a target skin surface. In some embodiments, the dimensions of the backing can be a 12 mm diameter circle. In some embodiments, the dimensions of the backing can be a 12 mm wide strip with a “handle” section of up to 12 mm length beyond the edge of the 12 mm×12 mm patch. In some embodiments, a 12 mm square polyester tape with an approximately 12 mm square extended “handle” can be used. In some embodiments, the backing can be larger, e.g., about 25 mm square, optionally with rounded corners. In some embodiments, the backing can be about a 25 mm diameter circle. In some embodiments, the area of backing that extends beyond the array can serve to hold the patch onto the skin with a biocompatible skin adhesive.

### Dissolvable Base

The microneedles and microneedle devices of the present disclosure generally comprise a base layer, such as a dissolving base layer. The base layer (e.g., dissolving base layer) forms the base of the needles (e.g., functions as the support for the distal microneedle tips, e.g., silk fibroin tips, that are loaded with a vaccine, an antigen, and/or an immunogen, e.g., a coronavirus vaccine and/or an influenza vaccine). The base layer (e.g., dissolvable base layer) can also function as a layer connecting adjacent needles to form a microneedle array or patch.

In some embodiments, the base layer (e.g., dissolving base layer) comprises a material that can dissolve into the subject, e.g., within the intended wear time (e.g., about five minutes). In some embodiments, at least about 10%, 20%, 30%, 40%, 50%, 60%, 70%, 80%, 90%, or 100% of the dissolvable base layer is dissolved after application to a biological barrier (e.g., skin) of a subject within the intended wear time (e.g., over a period of time of less than 1 hour, e.g., about 1 minute to about 45 minutes, about 2 minutes to about 30 minutes, about 5 minutes to about 15 minutes, e.g., about 1 minute, about 2 minutes, about 3 minutes, about 4 minutes, about 5 minutes, about 6 minutes, about 7 minutes, about 8 minutes, about 9 minutes, or about 10 minutes or more). In some embodiments, at least about 10%, 20%, 30%, 40%, 50%, 60%, 70%, 80%, 90%, or 100% of the dissolvable base layer is dissolved after application to a biological barrier (e.g., skin) of a subject within about 5 minutes.

In some embodiments, the material used in the fabrication of the dissolvable base is sufficiently strong enough to enable the microneedle to penetrate the skin, and is tough enough (e.g., not excessively brittle) to also enable demolding of the microneedle during fabrication. In some embodiments, the dissolvable base material is amenable to routine handling without catastrophic failure, and retains its mechanical properties between demolding and application (e.g., not so hygroscopic that it melts due to ambient humidity). In some embodiments, the dissolvable base layer material is non-toxic and non-reactogenic at the doses used in a patch. In some embodiments, the dissolvable base layer comprises a water-soluble component.

Non-limiting examples of materials that may be used to fabricate the base layer (e.g., dissolvable base layer) include a polysaccharide, a disaccharide, a polymer, a protein, a plasticizer, and/or a surfactant. In some embodiments, the base layer (e.g., dissolving base layer) comprises one or more (e.g., two or more, three or more, four or more, five or more, or all) of a polysaccharide (e.g., dextran); a disaccharide (e.g., sucrose, maltose, and trehalose); a polymer (e.g., methyl cellulose, polyethylene glycol (PEG), carboxymethylcellulose (CMC), polyvinylpyrrolidone (PVP), polyvinyl alcohol (PVA), hyaluronate); a protein (e.g., gelatin, fibroin); a plasticizer (e.g., glycerol, propanediol); and a surfactant (e.g., a octyl phenol ethoxylate (e.g., Triton-X), a polysorbate, a poloxamers, and/or a polyethoxylated alcohol).

The base layers disclosed herein can comprise a polysaccharide, a disaccharide, a polymer, a protein, a plasticizer, and/or a surfactant at a concentration between about 0.001% and about 75% (e.g., between about 0.001% to about 1%, e.g., about 1%, about 2%, about 3%, about 4%, about 5%, about 6%, about 7%, about 8%, about 9%, about 10%, about 15%, about 20%, about 25%, about 30%, or about 35%, about 40%, about 45%, about 50%, about 55%, about 60%, about 65%, about 70%, or about 75%). In some instances, a dried solid base can comprise a polysaccharide, a disaccharide, a polymer, a protein, a plasticizer, and/or a surfactant at a concentration of up to about 100%. In some instances, a dried solid base can comprise a surfactant at a concentration of about 0.001%.

In some instances, the amount of a material that may be used to fabricate the base layer (e.g., dissolvable base layer) as described herein may refer to the amount present in a base layer solution (“print solution”) used during fabrication. In certain embodiments, this amount may be characterized as a volume/volume (v/v), weight/weight (w/w), or a weight/volume (w/v) measurement.

In some instances, the percentage (%) of a material that may be used to fabricate the base layer (e.g., dissolvable base layer) as described herein may refer to the percentage (%) present in a base layer solution (“print solution”) used during fabrication. In certain embodiments, this percentage (%) may be characterized as a volume/volume (v/v), weight/weight (w/w), or a weight/volume (w/v) measurement.

In some embodiments, the base layer (e.g., dissolving base layer) is configured for sustained release of a vaccine, an antigen, and/or an immunogen (e.g., a coronavirus vaccine and/or an influenza vaccine). In some embodiments, the base layer (e.g., dissolving base layer) comprises one or more (e.g., two or more, three or more, four or more, five or more, or six or more) of gelatin, dextran, glycerol, polyethylene glycol (PEG), sucrose, trehalose, maltose, carboxymethylcellulose (CMC), polyvinylpyrrolidone (PVP), polyvinyl alcohol (PVA), hyaluronate, methyl cellulose, and/or a surfactant (e.g., a octyl phenol ethoxylate (e.g., Triton-X), a polysorbate, a poloxamers, and/or a polyethoxylated alcohol), optionally wherein the microneedle is configured for sustained release.

In some embodiments, the base layer (e.g., dissolving base layer) comprises one or more (e.g., two or more, three or more, or four or more) of dextran, sucrose, glycerol, and a surfactant (e.g., a octyl phenol ethoxylate (e.g., Triton-X), a polysorbate, a poloxamers, and/or a polyethoxylated alcohol), optionally configured for sustained release.

In some embodiments, the base layer (e.g., dissolving base layer) is configured for burst release of a vaccine, an antigen, and/or an immunogen (e.g., a coronavirus vaccine and/or an influenza vaccine). In some embodiments, the base layer (e.g., dissolving base layer) comprises one or more (e.g., two or more, three or more, four or more, five or more, or six or more) of gelatin, dextran, glycerol, PEG, sucrose, trehalose, maltose, CMC, PVP, PVA, hyaluronate, methyl cellulose, and/or a surfactant (e.g., a octyl phenol ethoxylate (e.g., Triton-X), a polysorbate, a poloxamers, and/or a polyethoxylated alcohol), optionally wherein the microneedle is configured for burst release. In some embodiments, the base layer (e.g., dissolving base layer) comprises polyvinyl alcohol (PVA) and sucrose, optionally configured for burst release.

In some embodiments, the base layer (e.g., dissolving base layer) comprises a dextran. In some embodiments, the dextran can have a molecular weight of between about 30 kDa to about 600 kDa. In some embodiments, the dextran is about 40 kDa, about 50 kDa, about 60 kDa, about 70 kDa, about 80 kDa, about 90 kDa, about 100 kDa, about 200 kDa, about 300 kDa, about 400 kDa, about 500 kDa, or about 600 kDa. In some embodiments, a mixture of different dextrans can be used, e.g., a mixture of dextrans having various molecular weights. In some embodiments, the dextran can be obtained and/or derived from a variety of bacterial sources, including, but not limited to, Leuconostoc mesenteroides.

In some embodiments, the base layer (e.g., dissolving base layer) does not comprise poly(acrylic acid) (PAA). In some embodiments, a dissolvable base layer, as described herein, has improved biocompatibility, e.g., as compared to a dissolvable base layer comprising PAA. In some embodiments, the dissolvable base layer material causes a reduced inflammatory response and/or reduced tissue necrosis. In some embodiments, the dissolvable base layer material is not PAA, and induces a reduced inflammatory response and/or reduced tissue necrosis compared to PAA. In some embodiments, the dissolvable base layer material has a pH similar to that of the biological barrier into which it will be dissolved, e.g., a pH of about 4.0 to about 8.0.

In other embodiments, the base layer comprises a silk fibroin and/or a vaccine, an antigen, and/or an immunogen (e.g., a coronavirus vaccine and/or an influenza vaccine). The base layer (e.g., dissolvable base layer) can comprises less than 98% (e.g., less than about 98%, less than about 90%, less than about 80%, less than about 70%, less than about 60%, less than about 50%, less than about less 40%, less than about 30%, less than about 20%, less than about 10%, less than about 9%, less than about 8%, less than about 7%, less than about 6%, less than about 5%, less than about 4%, less than about 3%, less than about 2%, less than about 1%) of the total amount (e.g., dose) of a vaccine, antigen, and/or immunogen loaded into the microneedle and/or microneedle device.

In some embodiments, the base layer does not comprise, e.g., a detectable amount of, a silk fibroin and/or a vaccine, an antigen, and/or an immunogen. In some embodiments, the base layer is formulated to limit and/or reduce the amount of vaccine, antigen, and/or immunogen leakage (e.g., diffusion) from the microneedle tips (e.g., silk fibroin tips) into the base layer, e.g., as compared to art known base layer formulations, e.g., base layer formulations comprising PAA. In some embodiments, a limited and/or reduced amount of a vaccine, antigen, and/or immunogen leakage (e.g., diffusion) from the silk fibroin tips can be determined about 1 day, about 2 days, about 3 days, about 4 days, about 5 days, or about 6 days; about 1 week, about 2 weeks, or about 3 weeks; about 1 month, about 2 months, about 3 months, about 4 months, about 5 months, about 6 months, about 7 months, about 8 months, about 9 months, about 10 months, or about 11 months; or about 1 year or more after fabrication and storage (e.g., storage at about 4° C. (e.g., refrigeration), at about 25° C. (e.g., room temperature), at about 37° C. (e.g., body temperature), at about 45° C. and/or at about 50° C.), e.g., as compared to a base layer formulation comprising PAA.

In some embodiments, the dissolvable base comprises between about 10% and about 70% gelatin (e.g., hydrolyzed gelatin) (e.g., about 10%, about 20%, about 30%, about 40%, about 50%, about 60%, or about 70% gelatin).

In some embodiments, the dissolvable base comprises between about 10% and about 70% of a plasticizer, such as glycerol (e.g., about 10%, about 20%, about 30%, about 40%, about 50%, about 60%, or about 70% glycerol). In some embodiments a plasticizer is added to reduce brittleness.

In some embodiments, the dissolvable base comprises between about 0.001% and about 5% of a surfactant described herein, such as polysorbate (e.g., about 0.001% to about 1%, or about 1% to about 5% surfactant). In some embodiments a surfactant is added to aid in processing. In some embodiments, a surfactant is added as a plasticizer.

In some embodiments, the dissolvable base comprises between about 1% and about 70% polyethylene glycol (PEG) (e.g., about 1%, about 2%, about 3%, about 4%, about 5%, about 6%, about 7%, about 8%, about 9%, about 10%, about 20%, about 30%, about 40%, about 50%, about 60%, or about 70% PEG).

In some embodiments, the dissolvable base comprises between about 1% and about 35% sucrose (e.g., about 1%, about 2%, about 3%, about 4%, about 5%, about 6%, about 7%, about 8%, about 9%, about 10%, about 15%, about 20%, about 25%, about 30%, or about 35% sucrose).

In some embodiments, the dissolvable base comprises between about 1% and about 35% carboxymethylcellulose (CMC) (e.g., about 1%, about 2%, about 3%, about 4%, about 5%, about 6%, about 7%, about 8%, about 9%, about 10%, about 15%, about 20%, about 25%, about 30%, or about 35% CMC).

In some embodiments, the microneedle device (e.g., the microneedle tip and/or the base, e.g. the dissolvable base) does not comprise carboxymethyl cellulose, or if CMC is present, it is present at an amount of 35% w/w or less. In some embodiments, the microneedle (e.g., the microneedle tip and/or the base) comprises less than 35% w/w of carboxymethyl cellulose (e.g., less than 30% w/w, 29% w/w, 28% w/w, 27% w/w, 26% w/w, 25% w/w, 24% w/w, 23% w/w, 22% w/w/, 21% w/w, 20% w/w, 19% w/w, 18% w/w, 17% w/w, 16% w/w, 15% w/w, 14% w/w, 13% w/w, 12% w/w, 11% w/w, 10% w/w, 9% w/w, 8% w/w, 7% w/w, 6% w/w, 5% w/w, 4% w/w, 3% w/w, 2% w/w, 1% w/w, 0.5% w/w, 0.4% w/w, 0.3% w/w, 0.2% w/w, or 0.1% w/w of carboxymethyl cellulose).

In some embodiments, the dissolvable base comprises between about 10% and about 70% w/v polyvinylpyrrolidone (PVP) (e.g., about 10%, about 20%, about 30%, about 40%, about 50%, about 60%, or about 70% w/v PVP). In some embodiments, the PVP is 10 kDA PVP.

In some embodiments, the dissolvable base comprises between about 0.01% and about 5% v/v Triton X-100 (e.g., about 0.01%, about 0.05%, about 0.1%, about 0.15%, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, about 0.9%, about 0.95%, or about 1% v/v Triton X-100).

In some embodiments, the dissolvable base comprises between about 10% and about 70% w/v polyvinylpyrrolidone (PVP) (e.g., about 10%, about 20%, about 30%, about 40%, about 50%, about 60%, or about 70% w/v PVP) and between about 0.01% and about 5% v/v Triton X-100 (e.g., about 0.01%, about 0.05%, about 0.1%, about 0.15%, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, about 0.9%, about 0.95%, or about 1% v/v Triton X-100).

In some embodiments, the dissolvable base comprises between about 1% and about 35% polyvinyl alcohol (PVA) (e.g., e.g., about 1%, about 2%, about 3%, about 4%, about 5%, about 6%, about 7%, about 8%, about 9%, about 10%, about 15%, about 20%, about 25%, about 30%, or about 35% PVA).

In some embodiments, the dissolvable base comprises between about 1% and about 75% hyaluronate (e.g., about 1%, about 2%, about 3%, about 4%, about 5%, about 6%, about 7%, about 8%, about 9%, about 10%, about 15%, about 20%, about 25%, about 30%, or about 35%, about 40%, about 45%, about 50%, about 55%, about 60%, about 65%, about 70%, or about 75% hyaluronate).

In some embodiments, the dissolvable base comprises between about 1% and about 75% maltose (e.g., about 1%, about 2%, about 3%, about 4%, about 5%, about 6%, about 7%, about 8%, about 9%, about 10%, about 15%, about 20%, about 25%, about 30%, or about 35%, about 40%, about 45%, about 50%, about 55%, about 60%, about 65%, about 70%, or about 75% maltose).

In some embodiments, the dissolvable base comprises between about 1% and about 75% methyl cellulose (e.g., about 1%, about 2%, about 3%, about 4%, about 5%, about 6%, about 7%, about 8%, about 9%, about 10%, about 15%, about 20%, about 25%, about 30%, or about 35%, about 40%, about 45%, about 50%, about 55%, about 60%, about 65%, about 70%, or about 75% methyl cellulose).

In some embodiments, the dissolvable base layer comprises 40% hydrolyzed gelatin, 10% sucrose w/v in deionized (DI) water. Optionally, the base layer may include 1% low-viscosity carboxymethylcellulose (CMC), which can reduce brittleness. In some embodiments, the dissolvable base layer may comprise polyvinylpyrrolidone (PVP) of 10 kD MW at up to 50% w/v in DI water; polyvinyl alcohol (PVA) 87% hydrolyzed at 13 kD MW, at up to 20% in DI water; or CMC at up to 10% in DI water. The following combinations may also be suitable for use in the fabrication of a dissolvable base layer: 30% PVP and 10% PVA; 37% PVP, 5% PVA, and 15% sucrose; or various other proportions of PVP, PVA, and sucrose.

In some embodiments, the dissolvable base layer is approximately 12 mm square and 0.75 mm thick. In some embodiments, the dissolvable base layer can cover the entire patch. In some embodiments, the dimension of the base layer can be a 12 mm diameter circle, or a 12×12 mm square.

### Microneedle Tip

The microneedles and microneedle devices described herein comprise a microneedle tip. In some embodiments, the microneedle tip is an implantable microneedle tip. In some embodiments, the microneedle tip is a silk fibroin-based microneedle tip (e.g., an implantable microneedle tip). In some embodiments, the microneedle tip is an implantable sustained-release tip, e.g., comprising silk fibroin. The microneedle tip may further comprise a therapeutic agent, such as a vaccine, antigen, and/or immunogen (e.g., a coronavirus vaccine and/or an influenza vaccine) described herein, and optionally an additional therapeutic agent and/or adjuvant.

In some embodiments, the microneedle tip (e.g., silk fibroin tip) comprises a therapeutic agent as described herein. The methods provided herein can be used to fabricate microneedle tips, e.g., silk fibroin tips, e.g., implantable sustained-release tips, comprising a therapeutic agent in any suitable amount. For example, a microneedle tip may comprise between about 0.1 μg to about 50 μg of a therapeutic agent as described herein. In some embodiments, a microneedle tip may comprise at least about 0.1 μg, about 0.5 μg, about 1 μg, about 1.5 μg, about 2 μg, about 2.5 μg, about 3 μg, about 3.5 μg, about 4 μg, about 4.5 μg, about 5 μg, about 5.5 μg, about 6 μg, about 6.5 μg, about 7 μg, about 7.5 μg, about 8 μg, about 8.5 μg, about 9 μg, about 9.5 μg, about 10 μg, about 10.5 μg, about 11 μg, about 11.5 μg, about 12 μg, about 12.5 μg, about 13 μg, about 13.5 μg, about 14 μg, about 14.5 μg, about 15 μg, about 15.5 μg, about 16 μg, about 16.5 μg, about 17 μg, about 17.5 μg, about 18 μg, about 18.5 μg, about 19 μg, about 19.5 μg, about 20 μg, about 20.5 μg, about 21 μg, about 21.5 μg, about 22 μg, about 22.5 μg, about 23 μg, about 23.5 μg, about 24 μg, about 24.5 μg, about 25 μg, about 25.5 μg, about 26 μg, about 26.5 μg, about 27 μg, about 27.5 μg, about 28 μg, about 28.5 μg, about 29 μg, about 29.5 μg, about 30 μg, about 30.5 μg, about 31 μg, about 31.5 μg, about 32 μg, about 32.5 μg, about 33 μg, about 33.5 μg, about 34 μg, about 34.5 μg, about 35 μg, about 35.5 μg, about 36 μg, about 36.5 μg, about 37 μg, about 37.5 μg, about 38 μg, about 38.5 μg, about 39 μg, about 39.5 μg, about 40 μg, about 40.5 μg, about 41 μg, about 41.5 μg, about 42 μg, about 42.5 μg, about 43 μg, about 43.5 μg, about 44 μg, about 44.5 μg, about 45 μg, about 45.5 μg, about 46 μg, about 46.5 μg, about 47 μg, about 47.5 μg, about 48 μg, about 48.5 μg, about 49 μg, about 49.5 μg, or about 50 μg of a therapeutic agent as described herein. In some embodiments, the therapeutic agent comprises an mRNA as described herein. In some embodiments, the therapeutic agent comprises a vaccine, an antigen, and/or an immunogen (e.g., a coronavirus vaccine and/or an influenza vaccine) as described herein.

The methods provided herein can be used to fabricate microneedle tips, e.g., silk fibroin tips, e.g., implantable sustained-release tips, of any dimensions. The microneedle tips, e.g., silk fibroin tips, can be configured to comprise, and release, an effective amount of a vaccine, an antigen, and/or an immunogen (e.g., a coronavirus vaccine and/or an influenza vaccine). In some embodiments, the microneedle (e.g., microneedle tip) is configured to pierce a biological barrier (e.g., skin).

The microneedle tips described herein can include silk fibroin protein in any suitable amount. In some embodiments, a microneedle tip may comprise between about 0.1% and about 20% silk fibroin protein, or between about 0.1% and about 10% v/v silk fibroin protein, optionally, wherein the percentage (%) is based on the silk fibroin solution (“print solution”) used during fabrication. For example, a microneedle tip may comprise at least about 0.1%, about 0.25%, about 0.5%, about 0.75%, about 1%, about 1.25%, about 1.5%, about 1.75%, about 2%, about 2.25%, about 2.5%, about 2.75%, about 3%, about 3.25%, about 3.5%, about 3.75%, about 4%, about 4.25%, about 4.5%, about 4.75%, about 5%, about 5.25%, about 5.5%, about 5.75%, about 6%, about 6.25%, about 6.5%, about 6.75%, about 7%, about 7.25%, about 7.5%, about 7.75%, about 8%, about 8.25%, about 8.5%, about 8.75%, about 9%, about 9.25%, about 9.5%, about 9.75%, about 10%, about 10.5%, about 11%, about 11.5%, about 12%, about 12.5%, about 13%, about 13.5%, about 14%, about 14.5%, about 15%, about 15.5%, about 16%, about 16.5%, about 17%, about 17.5%, about 18%, about 18.5%, about 19%, about 19.5%, or about 20% v/v silk fibroin protein, optionally, wherein the percentage (%) is based on the silk fibroin solution (“print solution”) used during fabrication. In embodiments, a microneedle described herein can comprising a population of silk fibroin fragments having an average weight average molecular weight of between about 82 and about 92 kDa (e.g., about 82, about 83, about 84, about 85, about 86, about 87, about 88, about 89, about 90, about 91, or about 92 kDa).

In certain embodiments, the microneedle tip may comprise a surfactant in any suitable amount. In certain embodiments, the microneedle tip may comprise a surfactant in an amount between about 0.1% and about 10% surfactant. For example, the microneedle tip may comprise at least about 0.1%, about 0.25%, about 0.5%, about 0.75%, about 1%, about 1.25%, about 1.5%, about 1.75%, about 2%, about 2.25%, about 2.5%, about 2.75%, about 3%, about 3.25%, about 3.5%, about 3.75%, about 4%, about 4.25%, about 4.5%, about 4.75%, about 5%, about 5.25%, about 5.5%, about 5.75%, about 6%, about 6.25%, about 6.5%, about 6.75%, about 7%, about 7.25%, about 7.5%, about 7.75%, about 8%, about 8.25%, about 8.5%, about 8.75%, about 9%, about 9.25%, about 9.5%, about 9.75%, or about 10% surfactant, optionally, wherein the percentage (%) is based on the solution (“print solution”) used during fabrication. In certain embodiments, the surfactant may be Tween, such as Tween 20.

In some instances, the amount of a material that may be used to fabricate the microneedle tips as described herein may refer to the amount present in a solution (“print solution”) used during fabrication. In certain embodiments, this amount may be characterized as a volume/volume (v/v), weight/weight (w/w), or a weight/volume (w/v) measurement.

In some instances, the percentage (%) of a material that may be used to fabricate the microneedle tips as described herein may refer to the percentage (%) present in a solution (“print solution”) used during fabrication. In certain embodiments, this percentage (%) may be characterized as a volume/volume (v/v), weight/weight (w/w), or a weight/volume (w/v) measurement.

In some embodiments, the microneedle tips, e.g., silk fibroin tips have dimensions ranging from about 75 μm to about 800 μm in height/length (e.g., about 75, about 100 μm, about 125 μm, about 150 μm, about 250 μm to about 300 μm, about 300 μm to about 350 μm, about 350 μm to about 400 μm, about 400 μm to about 450 μm, about 450 μm to about 500 μm, about 500 μm to about 550 μm, about 550 μm to about 600 μm, about 600 μm to about 650 μm, about 650 μm to about 700 μm, about 700 μm to about 750 μm, about 750 μm, to about 800 μm). In certain embodiments, the microneedle tips, e.g., silk fibroin tips, have dimensions ranging from about 200 μm to about 500 μm in height/length. In certain embodiments, the microneedle tips, e.g., silk fibroin tips have dimensions ranging from about 300 μm to about 400 μm in height/length.

In some embodiments, the microneedle tip, e.g., silk fibroin tip, e.g., implantable tip, can have a diameter of any size, e.g., based upon the type of biological barrier (e.g., skin layer) intended to be pierced by the tip. In some embodiments, the microneedle tips, e.g., silk fibroin tips have a tip radius of about 10 μm or less (e.g., between about 1 μm and about 10 μm, e.g., about 1 μm or less, about 2 μm or less, about 3 μm or less, about 4 μm or less, about 5 μm or less, about 6 μm or less, about 7 μm or less, about 8 μm or less, about 9 μm or less, or about 10 μm or less). In embodiments, the tip can have a dimension (e.g., a diameter) ranging from about 50 nm to about 50 μm (e.g., about 50 nm to about 250 nm, about 250 nm to about 500 nm, about 500 to about 750 nm, about 750 nm to about 1 μm, about 1 μm to about 5 μm, about 5 μm to about 10 μm, about 10 μm to about 15 μm, about 15 μm to about 20 μm, about 20 μm to about 25 μm, about 25 μm to about 30 μm, about 30 μm to about 35 μm, about 35 μm to about 40 μm, about 40 μm to about 45 μm, or about 45 μm to about 50 μm). It can be understood that there is no fundamental limitation preventing the tips from having even smaller diameters (e.g., the limit of silk replica casting has been demonstrated with a resolution of tens of nm, see, e.g., Perry et al., Adv. Mat. (2008) 20:3070).

In some embodiments, the sharpness of the microneedle tip point (e.g., silk fibroin tip point), e.g., implantable sustained-release tip point, is described herein in terms of tip radius. The molds used in the fabrication of the microneedles described herein are designed to have a tip radius between about 0.5 μm to about 10 μm (e.g., about 0.5 μm, 0.6 μm, 0.7 μm, 0.8 μm, 0.9 μm, 1 μm, 2 μm, 3 μm, 4 μm, 5 μm, 6 μm, 7 μm, 8 μm, 9 μm, or 10 m). In some embodiments, the tip radius is between about 20 μm to about 25 μm (e.g., about 20 μm, 21 μm, 22 μm, 23 μm, 24 μm, or 25 μm). Without being bound by theory, it can be understood that blunter needles may require more force to penetrate the epidermis. In embodiments, other dimensions of the microneedle tip, e.g., silk fibroin tip, e.g., implantable sustained-release tip, may be controlled by the shape of the mold and/or fill volume. In some embodiments, the microneedle tip (e.g., silk fibroin tip), e.g., implantable sustained-release tip, can have an included angle between about 5 degrees and about 45 degrees (e.g., about 5, 6, 7, 8, 9, 10, 15, 20, 25, 30, 35, 40, or 45 degrees). In some embodiments, the microneedle tip (e.g., silk fibroin tip), e.g., implantable sustained-release tip, can have an included angle between about 15 degrees and 45 degrees (e.g., about 15 degrees, about 16 degrees, about 17 degrees, about 18 degrees, about 19 degrees, about 20 degrees, about 21 degrees, about 22 degrees, about 23 degrees, about 24 degrees, about 25 degrees, about 26 degrees, about 27 degrees, about 28 degrees, about 29 degrees, about 30 degrees, about 31 degrees, about 32 degrees, about 33 degrees, about 34 degrees, about 35 degrees, about 36 degrees, about 37 degrees, about 38 degrees, about 39 degrees, about 40 degrees, about 41 degrees, about 42 degrees, about 43 degrees, about 44 degrees, or about 45 degrees.

In embodiments, the height of the microneedle tip (e.g., silk fibroin tip), e.g., implantable sustained-release tip, may depend on the formulation and fill volume (e.g., fill volume or droplet dispensing volume), which can influence the surface tension and drying kinetics. In some embodiments, the height of the tip may extend to half of the full height of the microneedle. In some embodiments, the height of the microneedle tip (e.g., silk fibroin tip), e.g., implantable sustained-release tip, is between about 75 μm to about 475 μm (e.g., about 75, about 100 μm, about 125 μm, about 150 μm, about 175 μm, about 200 μm, about 225 μm, about 250 μm, about 275 μm, about 300 μm, about 325 μm, about 375 μm, about 400 μm, about 425 μm, or about 475 μm). In some embodiments, the height of the microneedle tip (e.g., silk fibroin tip), e.g., implantable sustained-release tip, is between about 300 μm to about 400 μm. In some embodiments, a portion of the tip comprises a thin “shell”-like layer roughly between about 5-10 μm thick (e.g., about 5, 6, 7, 8, 9, or 10 μm thick). In some embodiments, the microneedle tip (e.g., silk fibroin tip), e.g., implantable sustained-release tip, may dry to a more solid construct with a minimal “shell” wherein the height may be closer to 150 μm (e.g., between about 50 μm and about 200 μm) and the thickness >50 μm (e.g., between about 25 μm and about 75 μm).

Further, the microneedles of the present disclosure can take advantage of art known techniques developed, e.g., to functionalize silk fibroin (e.g., active agents such as dyes and sensors). See, e.g., U.S. Pat. No. 6,287,340, Bioengineered anterior cruciate ligament; WO 2004/000915, Silk Biomaterials & Methods of Use Thereof; WO 2004/001103, Silk Biomaterials & Methods of Use Thereof; WO 2004/062697, Silk Fibroin Materials & Use Thereof; WO 2005/000483, Method for Forming Inorganic Coatings; WO 2005/012606, Concentrated Aqueous Silk Fibroin Solution & Use Thereof; WO 20111005381, Vortex-Induced Silk fibroin Gelation for Encapsulation & Delivery; WO 20051123114, Silk-Based Drug Delivery System; WO 2006/076711, Fibrous Protein Fusions & Uses Thereof in the Formation of Advanced Organic/Inorganic Composite Materials; U.S. Application Pub. No. 2007/0212730, Covalently Immobilized Protein Gradients in Three-Dimensional Porous Scaffolds; WO 2006/042287, Method for Producing Biomaterial Scaffolds; WO 2007/016524, Method for Stepwise Deposition of Silk Fibroin Coatings; WO 2008/085904, Biodegradable Electronic Devices; WO 20081118133, Silk Microspheres for Encapsulation & Controlled Release; WO 2008/1108838, Microfluidic Devices & Methods for Fabricating Same; WO 2008/1127404, Nanopatterned Biopolymer Device & Method of Manufacturing Same; WO 2008/1118211, Biopolymer Photonic Crystals & Method of Manufacturing Same; WO 2008/1127402, Biopolymer Sensor & Method of Manufacturing Same; WO 2008/1127403, Biopolymer Optofluidic Device & Method of Manufacturing the Same; WO 2008/1127401, Biopolymer Optical Wave Guide & Method of Manufacturing Same; WO 2008/1140562, Biopolymer Sensor & Method of Manufacturing Same; WO 2008/1127405, Microfluidic Device with Cylindrical Microchannel & Method for Fabricating Same; WO 2008/1106485, Tissue-Engineered Silk Organs; WO 2008/1140562, Electroactive Biopolymer Optical & Electro-Optical Devices & Method of Manufacturing Same; WO 2008/1150861, Method for Silk Fibroin Gelation Using Sonication; WO 2007/1103442, Biocompatible Scaffolds & Adipose-Derived Stem Cells; WO 2009/1155397, Edible Holographic Silk Products; WO 2009/1100280, 3-Dimensional Silk Hydroxyapatite Compositions; WO 2009/061823, Fabrication of Silk Fibroin Photonic Structures by Nanocontact Imprinting; and WO 2009/1126689, System & Method for Making Biomaterial Structures.

In some embodiments, the microneedle tip (e.g., silk fibroin tip) comprises a vaccine, an antigen, and/or an immunogen (e.g., a coronavirus vaccine and/or an influenza vaccine) described herein. In some embodiments, the tip can be designed to be deployed into the dermis layer of the skin (e.g., not into the subcutaneous space), as the population of antigen presenting cells in the dermis is typically higher than in the subcutaneous space (e.g., skin-resident dendritic cells). In humans, the dermis ranges from about 1000-2000 m (e.g., about 1-2 mm) thick based on location and patient age and health. In rodents, the dermis is much thinner (e.g., mice ˜100-300 m, and rats ˜800-1200 m). Without wishing to be bound by theory, with a 650 m tall microneedle, a tip, e.g., an implantable sustained-release tip, may be deployed at a depth of between about 100 μm and about 600 μm to achieve the controlled- or sustained-release of a vaccine, an antigen, and/or an immunogen (e.g., a coronavirus vaccine and/or an influenza vaccine) as described herein.

In various embodiments, the microneedle tips (e.g., silk fibroin-based microneedle tips) further comprise at least one additional therapeutic agent, wherein the additional therapeutic agent can be dispersed throughout the microneedle or form at least a portion of the microneedle tip. In some embodiments, the additional therapeutic agent is useful in the treatment of a viral infection described herein. Optionally the silk fibroin-based microneedle tips can further comprise an excipient and/or adjuvant, as described herein.

Without being bound by theory, the molecular weight of the silk fibroin solution used in the fabrication of a microneedle described herein can function as a control factor to modulate the release of a vaccine, an antigen, and/or an immunogen (e.g., a coronavirus vaccine and/or an influenza vaccine) from the tip. In some embodiments, a higher molecular weight silk fibroin solutions can favor a slower controlled- or sustained-release (e.g., reducing the amount of an initial burst (e.g., the amount released on Day 0) by at least about 10% and then releasing additional vaccine, antigen, and/or immunogen over at least about the next 4 days). In some embodiments, the controlled- or sustained-release of a vaccine, antigen, and/or immunogen from the tip may be over at least about 4 days (e.g., about 4, 5, 6, 7, 8, 9, 10, 11, 12, 13, or 14 or more days, e.g., between about 5 days and 25 days, between about 10 days and about 20 days, between about 10 days and about 15 days, between about 4 days and about 15 days, or between about 14 days and 15 days, e.g., between about 1-2 weeks, about 1-3 weeks, or about 1-4 weeks). In some embodiments, the release occurs over about 1 week to about 2 weeks. In some embodiments, the release occurs over about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, or about 25 days. In some embodiments, the release occurs over about 10 days. In some embodiments, the release occurs over about 11 days. In some embodiments, the release occurs over about 12 days. In some embodiments, the release occurs over about 13 days. In some embodiments, the release occurs over about 14 days. In some embodiments, the release occurs over about 15 days. In some embodiments, the release occurs over about 16 days.

Alternatively, or in addition to comprising silk fibroin protein, in some embodiments, the microneedle (e.g., microneedle tip) comprises poly(lactic-co-glycolic acid) (PLGA). In some embodiments, the microneedle (e.g., microneedle tip) comprises polyglutamic acid (PGA). In some embodiments, the microneedle (e.g., microneedle tip) comprises polycaprolactone (PCL). In some embodiments, the microneedle (e.g., microneedle tip) comprises hyaluronic acid (HA) (e.g., crosslinked HA). In some embodiments, the microneedle (e.g., microneedle tip) comprises gelatin. In some embodiments, the microneedle (e.g., microneedle tip) comprises a surfactant, such as Tween, optionally, Tween 20. In some embodiments, the microneedle (e.g., microneedle tip) comprises transgenic and/or recombinant silk fibroin.

In some embodiments, the silk fibroin solution used in the fabrication of a microneedle described herein can comprise between about 0.1% and about 20% v/v silk fibroin protein, or between about 0.1% and about 10% v/v silk fibroin protein. For example, the silk fibroin solution used in the fabrication of a microneedle described herein can comprise at least about 0.1%, about 0.25%, about 0.5%, about 0.75%, about 1%, about 1.25%, about 1.5%, about 1.75%, about 2%, about 2.25%, about 2.5%, about 2.75%, about 3%, about 3.25%, about 3.5%, about 3.75%, about 4%, about 4.25%, about 4.5%, about 4.75%, about 5%, about 5.25%, about 5.5%, about 5.75%, about 6%, about 6.25%, about 6.5%, about 6.75%, about 7%, about 7.25%, about 7.5%, about 7.75%, about 8%, about 8.25%, about 8.5%, about 8.75%, about 9%, about 9.25%, about 9.5%, about 9.75%, about 10%, about 10.5%, about 11%, about 11.5%, about 12%, about 12.5%, about 13%, about 13.5%, about 14%, about 14.5%, about 15%, about 15.5%, about 16%, about 16.5%, about 17%, about 17.5%, about 18%, about 18.5%, about 19%, about 19.5%, or about 20% silk fibroin protein. In embodiments, the silk fibroin solution used in the fabrication of a microneedle described herein can comprising a population of silk fibroin fragments having an average weight average molecular weight of between about 82 and about 92 kDa (e.g., about 82, about 83, about 84, about 85, about 86, about 87, about 88, about 89, about 90, about 91, or about 92 kDa).

In some embodiments, the silk fibroin solution used in the fabrication of a microneedle described herein can comprises a surfactant. For example, the silk fibroin solution used in the fabrication of a microneedle described herein can comprise at least about 0.1%, about 0.25%, about 0.5%, about 0.75%, about 1%, about 1.25%, about 1.5%, about 1.75%, about 2%, about 2.25%, about 2.5%, about 2.75%, about 3%, about 3.25%, about 3.5%, about 3.75%, about 4%, about 4.25%, about 4.5%, about 4.75%, about 5%, about 5.25%, about 5.5%, about 5.75%, about 6%, about 6.25%, about 6.5%, about 6.75%, about 7%, about 7.25%, about 7.5%, about 7.75%, about 8%, about 8.25%, about 8.5%, about 8.75%, about 9%, about 9.25%, about 9.5%, about 9.75%, or about 10% surfactant. In some embodiments, the surfactant may be Tween, such as Tween 20. In some instances, the percentage (%) of a material, such as a surfactant, used to fabricate the microneedle tips as described herein may be characterized as a volume/volume (v/v), weight/weight (w/w), or a weight/volume (w/v) measurement.

In embodiments, the silk fibroin solution used in the fabrication of a microneedle described herein can be a low molecular weight silk fibroin composition comprising a population of silk fibroin fragments having a range of molecular weights, characterized in that: no more than 15% of the total number of silk fibroin fragments in the population has a molecular weight exceeding 200 kDa, and at least 50% of the total number of the silk fibroin fragments in the population has a molecular weight within a specified range, wherein the specified range is between about 3.5 kDa and about 120 kDa, or between about 5 kDa and about 125 kDa. Stated another way, the silk fibroin solution used in the fabrication of a microneedle described herein can comprise a population of silk fibroin fragments having a range of molecular weights, characterized in that: no more than 15% of the total moles of silk fibroin fragments in the population has a molecular weight exceeding 200 kDa, and at least 50% of the total moles of the silk fibroin fragments in the population has a molecular weight within a specified range, wherein the specified range is between about 3.5 kDa and about 120 kDa, or between about 5 kDa and about 125 kDa (see, e.g., WO2014/145002, which is incorporated herein by reference herein).

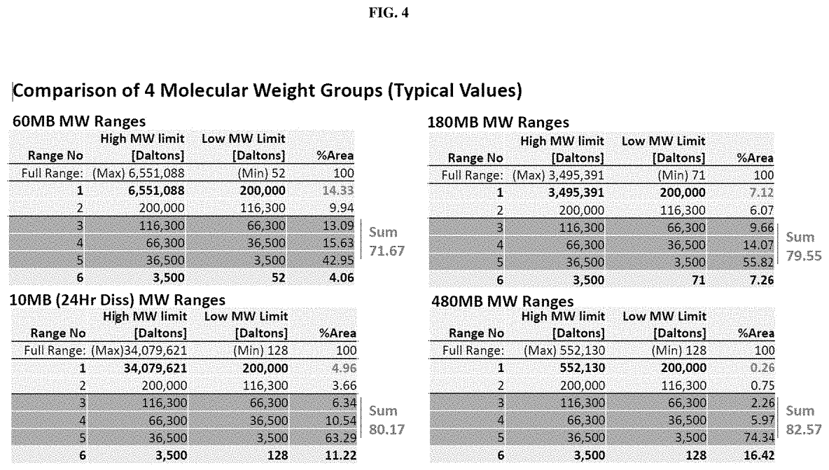

Exemplary silk fibroin (e.g., regenerated silk fibroin) solutions may have different molecular weight profiles, e.g., as determined by size exclusion chromatography (SEC) methods (see, e.g., FIG. 4). In some embodiments, the silk fibroin solutions can be prepared, e.g., according to established methods. In some embodiments, pieces of cocoons from the silkworm Bombyx mori are first boiled in 0.02 M Na₂CO₃ to remove sericin protein which is present in unprocessed, natural silk, prior to analysis by SEC. In some embodiments, silk fibroin composition can be a composition or mixture produced by degumming cocoons from the silkworm Bombyx mori at an atmospheric boiling temperature for about 480 minutes or less, e.g., less than 480 minutes, less than 400 minutes, less than 300 minutes, less than 200 minutes, less than 180 minutes, less than 120 minutes, less than 100 minutes, less than 60 minutes, less than 50 minutes, less than 40 minutes, less than 30 minutes, less than 20 minutes, less than 10 minutes or shorter. In one embodiment, the silk fibroin composition can be a composition or mixture produced by degumming silk cocoon at an atmospheric boiling temperature in an aqueous sodium carbonate solution for about 480 minutes or less, e.g., less than 480 minutes, less than 400 minutes, less than 300 minutes, less than 200 minutes, less than 180 minutes, less than 120 minutes, less than 100 minutes, less than 60 minutes, less than 50 minutes, less than 40 minutes, less than 30 minutes, less than 20 minutes, less than 10 minutes or shorter.

In some embodiments, the silk fibroin solution may be a 10-minute boil (10 MB), a 60-minute boil (60 MB), a 120-minute boil (120 MB), a 180-minute boil (180 MB), or a 480-minute boil (480 MB) silk fibroin solution (see, e.g., FIG. 4). In some embodiments, a vaccine, an antigen, and/or an immunogen (e.g., a coronavirus vaccine and/or influenza vaccine) can be formulated in a 1% w/v to about 10% w/v (e.g., about 1, 2, 3, 4, 5, 6, 7, 8, 9, or 10% w/v) 10 MB silk fibroin solution. In some embodiments, a vaccine, an antigen, and/or an immunogen (e.g., a coronavirus vaccine and/or influenza vaccine), can be formulated in a 1% w/v to about 10% w/v (e.g., about 1, 2, 3, 4, 5, 6, 7, 8, 9, or 10% w/v) 60 MB silk fibroin solution. In some embodiments, a vaccine, an antigen, and/or an immunogen (e.g., a coronavirus vaccine and/or influenza vaccine), can be formulated in a 1% w/v to about 10% w/v (e.g., about 1, 2, 3, 4, 5, 6, 7, 8, 9, or 10% w/v) 120 MB silk fibroin solution. In some embodiments, a vaccine, an antigen, and/or an immunogen (e.g., a coronavirus vaccine and/or influenza vaccine), can be formulated in a 1% w/v to about 10% w/v (e.g., about 1, 2, 3, 4, 5, 6, 7, 8, 9, or 10% w/v) 180 MB silk fibroin solution. In some embodiments a vaccine, an antigen, and/or an immunogen (e.g., a coronavirus vaccine and/or influenza vaccine), can be formulated in a 1% w/v to about 10% w/v (e.g., about 1, 2, 3, 4, 5, 6, 7, 8, 9, or 10% w/v) 480 MB silk fibroin solution.

Without being bound by theory, the primary tunability of the silk fibroin tip, e.g., implantable sustained-release tip, is its crystallinity, measured via beta-sheet content (intermolecular and intramolecular β-sheet). This impacts the solubility of the silk tip matrix and the ability of vaccine, antigen, and/or immunogen to be retained. With the increased 3-sheet content, the tip also becomes more mechanically strong. Specific vaccine, antigen, and/or immunogen (e.g., coronavirus vaccine and/or influenza vaccine) release profiles can be achieved through modulation of the crystallinity and the diffusivity of the silk matrix. This is accomplished through both silk input material and formulation as well as post-treatment to increase crystallinity (e.g., annealing, such as water annealing, or methanol/solvent annealing). In some embodiments, the silk fibroin tip, e.g., implantable controlled- or sustained-release microneedle tip, comprises a beta-sheet content of between about 10% and about 60% (e.g., about 10%, about 20%, about 30%, about 40%, about 50%, about 60%), e.g., as based on a “crystallinity index, “e.g., a “crystallinity index” known in the art. In some embodiments, the silk fibroin tip, e.g., implantable controlled- or sustained-release microneedle tip, can be formulated as a particle (e.g., a microparticle and/or a nanoparticle).

### Microneedle Dimensions

In some embodiments, the present disclosure provides microneedles (e.g., silk fibroin-based microneedles), and devices comprising the same, that have various dimensions and geometries.

In embodiments, the length of the microneedles (e.g., silk fibroin-based microneedles) can be fabricated sufficiently long enough to enable delivery (e.g., implantation) of a microneedle tip (e.g., silk fibroin tip) comprising a vaccine, an antigen, and/or an immunogen (e.g., a coronavirus vaccine and/or an influenza vaccine), and optionally an additional therapeutic agent, to a desired depth within a biological barrier (e.g., skin), e.g., to induce an immune response.

In some embodiments, the length of the microneedle (e.g., silk fibroin-based microneedle) is between about 350 μm to about 1500 μm (e.g., about 350 μm, about 400 μm, about 450 μm, about 500 μm, about 550 μm, about 600 μm, about 650 μm, about 700 μm, about 750 μm, about 800 μm, about 850 μm, about 900 μm, about 950 μm, about 1000 μm, about 1050 μm, about 1100 μm, about 1150 μm, about 1200 μm, about 1250 μm, about 1300 μm, about 1350 μm, about 1400 μm, about 1450 μm, about 1500 μm).

In some embodiments, the microneedle (e.g., silk fibroin-based microneedle) length is sufficient to enable delivery of an implantable tip, e.g., comprising a vaccine, an antigen, and/or an immunogen, to the epidermis (e.g., about 10 μm to 120 μm below the skin surface), e.g., to elicit an immune response. In some embodiments, the microneedle (e.g., silk fibroin-based microneedle) length is sufficient to enable delivery of an implantable tip, e.g., comprising a vaccine, an antigen, and/or an immunogen, to the dermis (e.g., about 60 μm to about 2.1 mm below the skin surface), e.g., to elicit an immune response.

In some embodiments, the microneedle is configured to implant the microneedle tip (e.g., silk fibroin tip) into a biological barrier of a subject at a depth (e.g., a max penetration depth of the distal part of the tip) of between about 100 μm and about 600 μm. In some embodiments, the length of the microneedle is between about 350 μm to about 1500 μm. In some embodiments, the height of the microneedle tip (e.g., silk fibroin tip) may extend to approximately half of the full height of the microneedle.

Without wishing to be bound by theory, a skilled artisan can adjust the microneedle length (e.g., silk fibroin-based microneedle length) for a number of factors, including, without limitations, tissue thickness, e.g., skin thickness, (e.g., as a function of age, gender, location on body, subject species (e.g., human), drug delivery profile, diffusion properties of the vaccine, antigen, and/or immunogen (e.g., the ionic charge and/or molecule weight, and/or shape of the vaccine, antigen, and/or immunogen), or any combinations thereof.

However, without wishing to be bound by theory, with an approximately 650 m tall microneedle, a microneedle tip (e.g., a silk fibroin tip) may be deployed (e.g., implanted) at a depth of between about 100 μm and about 600 μm within the dermis layer of the skin to a subject to achieve release (e.g., sustained release) of the vaccine, antigen, and/or immunogen (e.g., coronavirus vaccine and/or influenza vaccine) from the microneedle tip (e.g., silk fibroin tip). In some embodiments, the microneedle may be about 800 m tall (e.g., between about 500 μm and 1200 μm tall).

Exemplary microneedles of the present disclosure are depicted in FIGS. 2 and 3.

### Microneedle Patches

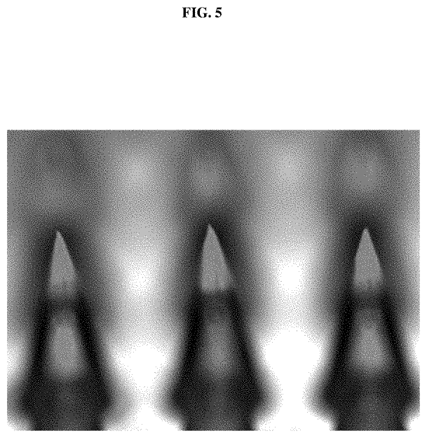

In some embodiments, a microneedle device (e.g., microneedle patch) of the present disclosure comprises a plurality of microneedles disclosed herein. The microneedle device (e.g., microneedle patch) can comprise a plurality of microneedles of the same type. Alternatively, the microneedle device can comprise a plurality of microneedles of different types. In another aspect of the present disclosure, the microneedles and microneedle devices (e.g., microneedle patches) described herein are manufactured by precision filling of each individual microneedle tip to enable different patterns of vaccine delivery, dosing schemes, and combined administration of different active agents, e.g., vaccines, antigens, immunogens, adjuvants, and/or additional therapeutic agents, described herein. For example, separate formulations of antigens may be distributed into different microneedle tips of a patch, for co-delivery of different antigens by the same patch without co-formulation of the antigens. An enlarged view of a portion of an exemplary microneedle device of the present disclosure, fabricated to include different formulations in different microneedles, is shown in FIG. 5.

The methods of immunization, vaccine delivery, and dosing described herein may comprise combination administration of a vaccine, antigen, and/or immunogen with an additional active agent. In some embodiments, an additional active agent may be co-formulated in the same tip as a vaccine. Without wishing to be bound by theory, such a combination could include adjuvants to drive stronger cellular immune responses and/or mucosal responses. Moreover, additional antigens could be delivered for heterologous “prime/boost-like” immunization, e.g., primary immunization with an antigen from a first virus strain (e.g., coronavirus and/or influenza virus), and a boost (e.g., provided via controlled- or sustained-release or a distinct kinetic pattern from “prime”) with a different antigen (e.g., an antigen of a drifted strain). For example, primary immunization may comprise an HA antigen from various influenza strains, and a boost (e.g., provided via controlled- or sustained-release or distinct kinetic pattern from “prime”) with a different antigen (e.g., a drifted strain, a hemagglutinin stem, m2e protein, or NA).

A microneedle device (e.g., silk fibroin-based microneedle device), e.g., microneedle patch, of the present disclosure can comprises a monovalent or multivalent vaccine, e.g., a bivalent, a trivalent, a quadrivalent (or tetravalent), or a pentavalent vaccine. In some embodiments, the microneedle device comprises a quadrivalent vaccine. In some embodiments, the microneedle device comprises a pentavalent vaccine. As described herein, the pentavalent vaccine may comprise one or more coronavirus antigen (e.g., one or more of a SARS-CoV-2 antigen, a SARS-CoV antigen, and/or a MERS-CoV antigen) and or more or more influenza antigen (e.g., one or more of an influenza A antigen, an influenza B antigen, an influenza C antigen, and/or an influenza D antigen).

Each component of a multivalent vaccine, e.g., comprising two, three, four, five, or more different antigens, may be co-formulated together in the same microneedle. For example, each microneedle of a device can comprise a co-formulation comprising all antigens of a quadrivalent or pentavalent vaccine. Alternatively, different microneedles of a device may comprise a different formulation to each other, e.g., comprising one or more different antigens. For example, different components of a multivalent vaccine (e.g., different coronavirus and/or influenza virus antigens) may be disposed in different microneedles of the device. The multivalent vaccine (e.g., quadrivalent or pentavalent vaccine) may comprise a combination of coronavirus antigens and/or influenza virus antigens. In some embodiments, the microneedle device, or plurality of microneedles, comprises a multivalent influenza vaccine (e.g., a bivalent, trivalent, quadrivalent (or tetravalent), or pentavalent influenza vaccine). In some embodiments, the microneedle device, or plurality of microneedles, comprises a quadrivalent influenza vaccine.

For example, in a microneedle device comprising multiple different antigens, e.g., a combination of one or more coronavirus and one or more influenza virus antigens, each of the different antigens may be individually formulated into its own set of microneedles in the device. In some embodiments, in a microneedle device comprising multiple different antigens, more than one of the different antigens are co-formulated together in the same set of microneedles, while one or more other antigen is formulated into a different set of microneedles of the device. For example, in a microneedle device comprising multiple different antigens, e.g., one or more coronavirus antigen and one or more influenza virus antigens, the one or more influenza virus antigens may be co-formulated into the same set of microneedles of the device, while the one or more coronavirus antigen is formulated into its own different set of microneedles of the device.

In some embodiments, the microneedle device comprises at least two microneedles that do not comprise the same formulation of vaccine, antigen, and/or immunogen as each other. In some embodiments, the microneedle device comprises at least three microneedles that do not comprise the same formulation of vaccine, antigen, and/or immunogen as each other. In some embodiments, the microneedle device comprises at least four microneedles that do not comprise the same formulation of vaccine, antigen, and/or immunogen as each other. In some embodiments, the microneedle device comprises at least five microneedles that do not comprise the same formulation of vaccine, antigen, and/or immunogen as each other.

A microneedle device (e.g., a microneedle patch) described herein may comprise a preselected distribution of one or more microneedles, comprising a coronavirus antigen and/or an influenza antigen. For example, a portion of the plurality of microneedles (e.g., 20% of the microneedles) may contain one or more coronavirus antigens, and the remaining portion (e.g., 80% of the microneedles) may contain one or more influenza antigens. In some embodiments, a portion of the plurality of microneedles (e.g., 20% of the microneedles) comprises one or more coronavirus antigens, and no influenza antigens; and the remaining portion of the plurality (e.g., 80% of the microneedles) comprises one or more influenza antigens, and no coronavirus antigen.

In some embodiments, the plurality of microneedles comprises at least 10%, 20%, 30%, 40%, 50% 60%, 70%, 80%, 90% or more (e.g., at least 20%) microneedles comprising one or more coronavirus antigens, e.g. one coronavirus antigen. In some embodiments, the plurality of microneedles comprises at least 10%, 20%, 30%, 40%, 50% 60%, 70%, 80%, 90% or more (e.g., at least 80%) microneedles comprising one or more influenza antigens, e.g., one, two, three or four influenza antigens.

In some embodiments, the plurality of microneedles comprises at least 10% microneedles which comprise one or more coronavirus antigens, e.g., one coronavirus antigen, and at least 90% microneedles which comprise one or more influenza antigens, e.g., one, two, three or four influenza antigens. In some embodiments, the plurality of microneedles comprises at least 20% microneedles which comprise one or more coronavirus antigens, e.g., one coronavirus antigen, and at least 80% microneedles which comprise one or more influenza antigens, e.g., one, two, three or four influenza antigens. In some embodiments, the plurality of microneedles comprises at least 30% microneedles which comprise one or more coronavirus antigens, e.g., one coronavirus antigen, and at least 70% microneedles which comprise one or more influenza antigens, e.g., one, two, three or four influenza antigens. In some embodiments, the plurality of microneedles comprises at least 40% microneedles which comprise one or more coronavirus antigens, e.g., one coronavirus antigen, and at least 60% microneedles which comprise one or more influenza antigens, e.g., one, two, three or four influenza antigens. In some embodiments, the plurality of microneedles comprises at least 50% microneedles which comprise one or more coronavirus antigens, e.g., one coronavirus antigen, and at least 50% microneedles which comprise one or more influenza antigens, e.g., one, two, three or four influenza antigens. In some embodiments, the plurality of microneedles comprises at least 60% microneedles which comprise one or more coronavirus antigens, e.g., one coronavirus antigen, and at least 40% microneedles which comprise one or more influenza antigens, e.g., one, two, three or four influenza antigens. In some embodiments, the plurality of microneedles comprises at least 70% microneedles which comprise one or more coronavirus antigens, e.g., one coronavirus antigen, and at least 30% microneedles which comprise one or more influenza antigens, e.g., one, two, three or four influenza antigens. In some embodiments, the plurality of microneedles comprises at least 80% microneedles which comprise one or more coronavirus antigens, e.g. one coronavirus antigen, and at least 20% microneedles which comprise one or more influenza antigens, e.g., one, two, three or four influenza antigens. In some embodiments, the plurality of microneedles comprises at least 90% microneedles which comprise one or more coronavirus antigens, e.g., one coronavirus antigen, and at least 10% microneedles which comprise one or more influenza antigens, e.g., one, two, three or four influenza antigens.

In some embodiments, the microneedle device comprises a first, second, third, fourth, and/or fifth microneedle comprising: (i) a coronavirus vaccine, antigen, and/or immunogen (e.g., a SARS-CoV-2 vaccine, a SARS-CoV vaccine, and/or a MERS-CoV vaccine); and (ii) an influenza vaccine, antigen, and/or immunogen (e.g., an influenza A vaccine, an influenza B vaccine, an influenza C vaccine, and/or an influenza D vaccine). In some embodiments, a microneedle of the device comprises only (i). In some embodiments, a microneedle of the device comprises only (ii). In some embodiments, a microneedle of the device comprises both (i) and (ii). In some embodiments, each of the first, second, third, fourth, and/or fifth microneedle comprises both (i) and (ii).

In some embodiments, the first microneedle comprises only (i), and the second microneedle comprises only (ii). In some embodiments, the third microneedle comprises only (ii), wherein the influenza vaccine, antigen, and/or immunogen is different from the influenza vaccine, antigen, and/or immunogen present in the second microneedle. In some embodiments, the influenza vaccine, antigen, and/or immunogen present in the third microneedle is the same as the influenza vaccine, antigen, and/or immunogen present in the second microneedle. In some embodiments, the fourth microneedle comprises only (ii), wherein the influenza vaccine, antigen, and/or immunogen is different from the influenza vaccine, antigen, and/or immunogen present in the second and/or third microneedle. In some embodiments, the influenza vaccine, antigen, and/or immunogen present in the fourth microneedle is the same as the influenza vaccine, antigen, and/or immunogen present in the second and/or third microneedle. In some embodiments, the fifth microneedle comprises only (ii), wherein the influenza vaccine, antigen, and/or immunogen is different from the influenza vaccine, antigen, and/or immunogen present in the second, third, and/or fourth microneedle. In some embodiments, the influenza vaccine, antigen, and/or immunogen present in the fifth microneedle is the same as the influenza vaccine, antigen, and/or immunogen present in the second, third, and/or fourth microneedle.

In some embodiments, the combination of vaccines, antigens, and/or immunogens present in the first, second, third, fourth, and/or fifth microneedle comprises a bivalent vaccine. In some embodiments, the combination of vaccines, antigens, and/or immunogens present in the first, second, third, fourth, and/or fifth microneedle comprises a trivalent vaccine. In some embodiments, the combination of vaccines, antigens, and/or immunogens present in the first, second, third, fourth, and/or fifth microneedle comprises a quadrivalent vaccine (e.g., a quadrivalent influenza vaccine). In some embodiments, the combination of vaccines, antigens, and/or immunogens preset in the first, second, third, fourth, and/or fifth microneedle comprises a pentavalent vaccine. In some embodiments, the first microneedle comprises a monovalent vaccine (e.g., a coronavirus vaccine). In some embodiments, the combination of vaccines present in the second, third, fourth, and fifth microneedle comprises a quadrivalent vaccine (e.g., a quadrivalent influenza vaccine). In some embodiments, each of the second, third, fourth, and fifth microneedles independently comprises a quadrivalent vaccine (e.g., a quadrivalent influenza vaccine). In some embodiments, the combination of the coronavirus vaccine present in the first microneedle, and the influenza vaccines present in the second, third, fourth, and fifth microneedles comprises a pentavalent vaccine.

Release Kinetics

The microneedles and microneedle devices (e.g., microneedle patches) described herein can be configured to release a vaccine, antigen, and/or immunogen (e.g., a coronavirus vaccine and/or an influenza vaccine), and optionally an additional therapeutic agent, according to various release kinetics. In some embodiments, the release kinetics mimic that of a natural infection (e.g., a viral infection), which can drive a more potent immune response (e.g., a more potent cellular and/or humoral immune response).

Without wishing to be bound by theory, the microneedles and microneedle devices described herein can mimic the natural process of antigen presentation (e.g., viral antigen presentation) by enabling the release, e.g., controlled- or sustained-release, of a virus-derived antigen, immunogen, and/or vaccine into a subject, e.g., into the dermis skin layer of a subject. The controlled- or sustained-release enabled by the formulations, compositions, articles, devices, and preparations, microneedles, and microneedle devices described herein can induce greater immunogenicity, an enhanced immune response (e.g., a more potent cellular and/or humoral immune response), and/or broad-spectrum immunity in a subject, as compared to the administration of single-dose or bolus administration of, e.g., a vaccine, such as an influenza vaccine.

The microneedle, e.g., the microneedle tip can slowly releases the vaccine, antigen, and/or immunogen over a time period sufficiently long enough to provide immunity to a virus (e.g., over a time period of at least about 4 days (e.g., about 1, 2, 3, 4, 5, 6, 7, 8, 9, 10, 11, 12, 13, or 14 or more days, e.g., between about 5 days about 25 days, between about 10 days and about 20 days, between about 10 days and about 15 days, between about between about 4 days and about 14 days, between about 12 days and 16 days, between about 14 days and 15 days e.g., between about 1-2 weeks, about 1-3 weeks, or about 1-4 weeks, e.g., about one week, about two weeks, about three weeks, about four weeks, about five weeks, or about six weeks or more weeks). In some embodiments, the microneedle tip slowly releases the vaccine, antigen, and/or immunogen (e.g., the coronavirus vaccine and/or influenza vaccine) over a period of about 5 days to about 25 days. In some embodiments, the microneedle tip slowly releases the vaccine, antigen, and/or immunogen (e.g., the coronavirus vaccine and/or influenza vaccine) over a period of about 10 days to about 20 days. In some embodiments, the microneedle tip slowly releases the vaccine, antigen, and/or immunogen (e.g., the coronavirus vaccine and/or influenza vaccine) over a period of about 10 days to about 15 days.

Various properties of implantable controlled- or sustained-release microneedle tips, e.g., microneedle tips comprising silk fibroin, can be modulated to tune (e.g., alter and/or modify) the release kinetics (e.g., rate of release) of a vaccine, an antigen, and/or an immunogen from the microneedle tip. For example, the crystallinity, beta-sheet content, and molecular weight of the silk fibroin can be modulated. In some embodiments, the implantable controlled- or sustained-release microneedle tip comprises a beta-sheet content of between about 10% and about 60% (e.g., about 10%, about 20%, about 30%, about 40%, about 50%, about 60%), e.g., as based on a “crystallinity index,” e.g., a “crystallinity index” known in the art.

In some embodiment, microneedles (e.g., silk fibroin-based microneedles) are configured to release a vaccine, antigen, and/or immunogen (e.g., a coronavirus vaccine and/or an influenza vaccine) by sustained release. Examples of sustained release include, but are not limited to, zero order release, first order release, and second order release. In some embodiments, zero order release is a rate of release that is independent of the vaccine, antigen, and/or immunogen concentration in the dosage form (e.g., microneedle). In some embodiments, zero order release is a release of a vaccine, antigen, and/or immunogen that is approximately constant over a period of time (e.g., a constant amount of a vaccine, antigen, and/or immunogen is released per unit time). In some embodiments, first order release is a rate of release that is a function of the amount of the vaccine, antigen, and/or immunogen remaining in the dosage form (e.g., microneedle). In some embodiments, first order release is a release of a constant proportion, such as a percentage, of a vaccine, antigen, and/or immunogen from the dosage form (e.g., microneedle) per unit time. In some embodiments, second order release is where doubling the concentration of a vaccine, antigen, and/or immunogen in the dosage form quadruples the release rate.

In some embodiments, sustained-released comprises a substantially continuous low dose administration of the vaccine, antigen, and/or immunogen (e.g., coronavirus vaccine and/or influenza vaccine). The sustained release can comprise a continuous administration of about a greater than 0% portion to about a 100% portion (e.g., about 1 to about 25%, about 25% to about 50%, about 50% to about 75%, about 75% to about 100%) of a total amount of a vaccine, antigen, and/or immunogen (e.g., a coronavirus vaccine and/or an influenza vaccine) present in the microneedle tip (e.g., silk fibroin tip). In some embodiments, the sustained release is over a period of time comprising at least about 4 days (e.g., between about 4 and 25 days, between about 5 and 25 days, between about 10 and 20 days, between about 10 days and about 15 days, between about 12 and 18 days, between about 14 and 16 days, or between about 14 and 15 days, e.g., about 4, 5, 6, 7, 8, 9, 10, 11, 12, 13, 14, 15, 16, 17, 18, 19, 20, 21, 22, 23, 24, or 25 or more days, e.g., between about 1 to about 2 weeks, between about 1 to about 3 weeks, or between about 2 to about 4 weeks, e.g., between about 1 to about 3 months, e.g., between about 2 to about 4 months, e.g., between about 3 to about 6 months). In certain embodiments, the sustained release is over a period of time between about 5 days and about 25 days. In certain embodiments, the sustained release is over a period of time between about 7 days and about 15 days. In certain embodiments, the sustained release is over a period of time between about 10 days and about 20 days. In certain embodiments, the sustained release is over a period of time between about 10 days and about 15 days.

In some embodiment, microneedles, e.g., silk fibroin-based microneedles, described herein are configured to release a vaccine, antigen, and/or immunogen (e.g., a coronavirus vaccine and/or an influenza vaccine) by burst release. In some embodiments, burst release comprises a rapid administration of the vaccine, antigen, and/or immunogen to the subject. The burst release can comprise a rapid administration of a greater than 0% portion to about a 100% portion (e.g., about 1 to about 25%, about 25% to about 50%, about 50% to about 75%, about 75% to about 100%) of a total amount of vaccine, antigen, and/or immunogen in the microneedle tip (e.g., silk fibroin tip). In some embodiments, the burst release is over a period of time comprising at least about 1 hour (e.g., about 1 to about 30 minutes, e.g., about 1, 2, 3, 4, 5, 6, 7, 8, 9, 10, 15, 20, or 24 hours).

In some embodiment, the release (e.g., administration) of a vaccine, antigen, and/or immunogen (e.g., a coronavirus vaccine and/or an influenza vaccine) from a microneedle, e.g., a silk fibroin-based microneedle, described herein can be facilitated by the diffusion of the vaccine, antigen, and/or immunogen from the microneedle or a portion thereof.

In some embodiment, the release (e.g., administration) of a vaccine, antigen, and/or immunogen (e.g., a coronavirus vaccine and/or an influenza vaccine) from a microneedle, e.g., a silk fibroin-based microneedle, described herein can be facilitated by the degradation (e.g., protease mediated degradation) of the microneedle or a portion thereof.

In some embodiments, the release (e.g., administration) of a vaccine, antigen, and/or immunogen (e.g., a coronavirus vaccine and/or an influenza vaccine) from a microneedle, e.g., silk fibroin-based microneedle, described herein can be facilitated by the dissolution of the microneedle or a portion thereof.

Without wishing to be bound by theory, in microneedle devices comprising a coronavirus vaccine and an influenza vaccine, the release of the coronavirus vaccine can occur at substantially the same rate (e.g., concurrently) with the release of the influenza vaccine. In other embodiments, the release of the coronavirus vaccine can occur at a different rate than the release rate of the influenza vaccine, such that the coronavirus vaccine is release substantially before or substantially after the release of the influenza vaccine.

### Antigens and Formulations

The present disclosure provides, in some embodiments, the delivery, e.g., the controlled- or sustained-delivery, of vaccines, antigens, and/or immunogens, and optionally additional therapeutic agents, e.g., by a formulation, composition, articles, device, preparation, microneedle and/or microneedle device (e.g., a microneedle patch) described herein and/or according to a method described herein. The vaccines, antigens, and/or immunogens may be derived from, and/or be targeted to a particular virus, such as a virus that is a member of the Coronaviridae family and/or a member of the Orthomyxoviridae family, e.g., a coronavirus or influenza virus described herein.

In some embodiments, a vaccine, a microneedle, and/or a microneedle device (e.g., a microneedle patch) described herein may comprise a virus, such as an inactivated virus, or a live attenuated virus. In some embodiments, the vaccine, antigen, and/or immunogen comprises a nucleic acid (e.g., a DNA and/or RNA) derived from a virus (e.g., a coronavirus and/or influenza virus). In some embodiments, the vaccine, antigen, and/or immunogen comprises a nucleic acid (e.g., a DNA and/or RNA) encoding a viral protein or portion thereof (e.g., a coronavirus and/or influenza virus protein or portion thereof). In some embodiments, the vaccine, antigen, and/or immunogen comprises an mRNA encoding a viral protein or portion thereof (e.g., a coronavirus and/or influenza virus protein or portion thereof). In some embodiments, the vaccine, antigen, and/or immunogen comprises an amino acid (e.g., a peptide and/or protein) derived from a virus (e.g., a coronavirus and/or an influenza virus). In some embodiments, the influenza vaccine, antigen, and/or immunogen comprise an inactivated and/or a live attenuated virion, or split virion, of a coronavirus and/or an influenza virus. In some embodiments, the vaccine and/or the microneedle comprises a non-replicating viral antigen.

Coronavirus Vaccines

The present disclosure features a microneedle, and/or a microneedle device (e.g., a microneedle patch) comprising a coronavirus vaccine, antigen, and/or immunogen. Coronaviruses are enveloped RNA viruses, within the Coronaviridae family, and harbor a positive-sense single stranded RNA genome. The genome of coronaviruses generally encodes four major structural proteins, the spike (S), envelope (E), membrane (M), and nucleocapsid (N). Coronaviruses may be classified as alphacoronaviruses, betacoronaviruses, gammacoronaviruses, or deltacoronaviruses. For example, SARS-CoV-2, SARS-CoV, and MERS-CoV are examples of a betacoronavirus. Coronaviruses can infect humans, as well as other animals including mammals and birds, e.g., bats, cows, pigs, chickens, turkeys, ferrets, cats, dogs, and rabbits, and cows. Through frequent mutation and recombination events, coronaviruses can continuously change and may be subject to both antigenic drift and antigenic shift.

Coronaviruses known to infect humans include severe acute respiratory syndrome coronavirus (SARS-CoV), severe acute respiratory syndrome coronavirus 2 (SARS-CoV-2; also known as hCoV-19 and 2019-nCoV), Middle East respiratory syndrome coronavirus (MERS-CoV), human coronavirus 229E (HCoV-229E), human coronavirus NL63 (HCoV-NL63), human coronavirus OC43 (HCoV-OC43), and human coronavirus HKU1 (HCoV-HKU1). While each of these coronaviruses may infect and cause illness in humans, SARS-CoV-2, SARS-CoV, and MERS-CoV are known to be highly pathogenic to humans, and typically replicate in the lower respiratory tract causing severe illness, such as pneumonia. SARS-CoV is the causative agent of acute respiratory syndrome (SARS), SARS-CoV-2 is the causative agent of coronavirus disease 2019 (COVID-19), and MERS-CoV is the causative agent of Middle East respiratory syndrome (MERS). SARS-CoV-2 in particular is readily transmitted from human to human, and upon its initial outbreak in 2019 the virus rapidly spread from China to multiple continents, leading to a global pandemic.

In some embodiments, the coronavirus is SARS-CoV-2. In some embodiments, the coronavirus is SARS-CoV. In some embodiments, the coronavirus is MERS-CoV. In some embodiments, the coronavirus is HCoV-229E. In some embodiments, the coronavirus is HCoV-NL63. In some embodiments, the coronavirus is HCoV-OC43. In some embodiments, the coronavirus is HCoV-HKU1.

In some embodiments, the coronavirus is a SARS-CoV-2 variant (a drifted strain), for example, selected from the group consisting of B.1.525, B.1.526, B.1.526.1, B.1.617, B.1.617.1, B.1.617.2, B.1.617.3, P.2, B.1.1.7, B.1.351, B.1.427, B.1.429, or P.1.

Coronaviruses possess glycosylated spike (S) proteins on the outer surface, that can be used by the virus to gain entry to host cells. For example, the spike protein of SARS-CoV-2 is a trimeric class I fusion protein, and generally exists in a metastable prefusion conformation that can undergo substantial structural rearrangement to fuse the viral membrane with the host cell membrane. The SARS-CoV-2 spike protein comprises an N-terminal domain (NTD), and a receptor-binding domain (RBD) that is integral to engaging a host cell receptor to undergo fusion. Due in part to the critical function of the spike protein, it represents a key target for antibody-mediated neutralization and can inform vaccine and/or antigen development. The structure of the SARS-CoV-2 spike protein has been determined by cryo-electron microscopy (Wrapp et al., Science (2020) 367:1260-1263).

An exemplary spike protein (of SARS-CoV-2) amino acid sequence is provided below:

(SEQ ID NO: 1)
MFVFLVLLPLVSSQCVNLTTRTQLPPAYTNSFTRGVYYPDKVFRSSVLH
STQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHK
NNKSWMESEFRVYSSANNCTFEYVSQPFLMDLEGKQGNFKNLREFVFKN
IDGYFKIYSKHTPINLVRDLPQGFSALEPLVDLPIGINITRFQTLLALH
RSYLTPGDSSSGWTAGAAAYYVGYLQPRTFLLKYNENGTITDAVDCALD
PLSETKCTLKSFTVEKGIYQTSNFRVQPTESIVRFPNITNLCPFGEVFN
ATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNL
DSKVGGNYNYLYRLFRKSNLKPFERDISTEIYQAGSTPCNGVEGFNCYF
PLQSYGFQPTNGVGYQPYRVVVLSFELLHAPATVCGPKKSTNLVKNKCV
NFNFNGLTGTGVLTESNKKFLPFQQFGRDIADTTDAVRDPQTLEILDIT
PCSFGGVSVITPGTNTSNQVAVLYQDVNCTEVPVAIHADQLTPTWRVYS
TGSNVFQTRAGCLIGAEHVNNSYECDIPIGAGICASYQTQTNSPRRARS
VASQSIIAYTMSLGAENSVAYSNNSIAIPTNFTISVTTEILPVSMTKTS
VDCTMYICGDSTECSNLLLQYGSFCTQLNRALTGIAVEQDKNTQEVFAQ
VKQIYKTPPIKDFGGFNFSQILPDPSKPSKRSFIEDLLFNKVTLADAGF
IKQYGDCLGDIAARDLICAQKFNGLTVLPPLLTDEMIAQYTSALLAGTI
TSGWTFGAGAALQIPFAMQMAYRFNGIGVTQNVLYENQKLIANQFNSAI
GKIQDSLSSTASALGKLQDVVNQNAQALNTLVKQLSSNFGAISSVLNDI
LSRLDKVEAEVQIDRLITGRLQSLQTYVTQQLIRAAEIRASANLAATKM
SECVLGQSKRVDFCGKGYHLMSFPQSAPHGVVFLHVTYVPAQEKNFTTA
PAICHDGKAHFPREGVFVSNGTHWFVTQRNFYEPQUITTDNTFVSGNCD
VVIGIVNNTVYDPLQPELDSFKEELDKYFKNHTSPDVDLGDISGINASV
VNIQKEIDRLNEVAKNLNESLIDLQELGKYEQYIKWPWYIWLGFIAGLI
AIVMVTIMLCCMTSCCSCLKGCCSCGSCCKFDEDDSEPVLKGVKLHYT

In some embodiments, the spike protein of SARS-CoV-2 may comprise various protein substitutions, such as those identified in SARS-CoV-2 variants B.1.525 (e.g., A67V, 69del, 70del, 144del, E484K, D614G, Q677H, F888L); B.1.526 (e.g., (L5F*), T95I, D253G, (S477N*), (E484K*), D614G, (A701V*)); B.1.526.1 (e.g., D80G, 144del, F157S, L452R, D614G, (T791I*), (T859N*), D950H); B.1.617 (e.g., L452R, E484Q, D614G); B.1.617.1 (e.g., (T95I), G142D, E154K, L452R, E484Q, D614G, P681R, Q1071H); B.1.617.2 (e.g., T19R, (G142D), 156del, 157del, R158G, L452R, T478K, D614G, P681R, D950N); B.1.617.3 (e.g., T19R, G142D, L452R, E484Q, D614G, P681R, D950N); P.2 (e.g., E484K, (F565L*), D614G, V1176F); B.1.1.7 (e.g., 69del, 70del, 144del, (E484K*), (S494P*), N501Y, A570D, D614G, P681H, T716I, S982A, D1118H (K1191N*)); B.1.351 (e.g., D80A, D215G, 241del, 242del, 243del, K417N, E484K, N501Y, D614G, A701V); B.1.427 (e.g., L452R, D614G); B.1.429 (e.g., S13I, W152C, L452R, D614G); and P.1 (e.g., L18F, T20N, P26S, D138Y, R190S, K417T, E484K, N501Y, D614G, H655Y, T102), wherein (*) denotes that the substitution has been detected in some sequences but not all for the variant. In some embodiments, the spike protein of SARS-CoV-2 may comprise a L452R and/or E484K protein substitution.

The present disclosure features microneedle devices, and/or microneedles, comprising coronavirus vaccines. The coronavirus vaccines may comprise a SARS-CoV-2 antigen, a SARS-CoV antigen, a MERS-CoV antigen, or a combination thereof. The coronavirus vaccine may be one of, or a combination of, several vaccine types. For example, the coronavirus vaccine can be a DNA-based formulation, an RNA-based formulation, a recombinant subunit containing viral epitopes, an adenovirus-based vector, a purified inactivated virus, or a combination thereof.

In some embodiments, the coronavirus vaccine comprises a SARS-CoV-2 vaccine. In some embodiments, the coronavirus vaccine comprises a SARS-CoV vaccine. In some embodiments, the coronavirus vaccine comprises a MERS-CoV vaccine. In some embodiments, a SARS-CoV-2 vaccine comprises a SARS-CoV-2 virus (e.g., a live attenuated SARS-CoV-2 virus, or an inactivated SARS-CoV-2 virus), or a SARS-CoV-2 antigen, e.g., SARS-CoV-2 spike protein (e.g., SARS-CoV-2-S1) or a subunit thereof. In some embodiments, a SARS-CoV vaccine comprises a SARS-CoV virus (e.g., a live attenuated SARS-CoV virus, or an inactivated SARS-CoV virus), or a SARS-CoV antigen, e.g., SARS-CoV spike protein (e.g., SARS-CoV-S1) or a subunit thereof. In some embodiments, the MERS-CoV vaccine comprises MERS-CoV virus (e.g., live attenuated MERS-CoV virus, or an inactivated MERS-CoV virus), or a MERS-CoV antigen, e.g., MERS-CoV spike protein (e.g., MERS-CoV-S1) or a subunit thereof.

A coronavirus vaccine described herein may comprise a protein, e.g., a SARS-CoV-2 protein, a SARS-CoV protein, and/or a MERS-CoV protein. The protein may be a recombinant protein, e.g., a recombinant SARS-CoV-2 protein, a recombinant SARS-CoV protein, and/or a recombinant MERS-CoV protein. In some embodiments, the recombinant protein is a recombinant SARS-CoV-2 spike protein. In some embodiments, the recombinant protein is a recombinant SARS-CoV spike protein. In some embodiments, the recombinant protein is a recombinant MERS-CoV spike protein. In some embodiments, the coronavirus vaccine comprises a trimeric structure. In some embodiments, the coronavirus vaccine comprises a pre-fusion SARS-CoV-2 spike protein that comprises a trimeric structure.

In some embodiments, the coronavirus vaccine comprises a spike protein or subunit thereof, e.g., a SARS-CoV-2 spike protein or a subunit thereof, a SARS-CoV spike protein or a subunit thereof, or a MERS-CoV spike protein or a subunit thereof. In some embodiments, the coronavirus vaccine comprises a pre-fusion spike protein (e.g., a pre-fusion SARS-CoV-2 spike protein, a pre-fusion SARS-CoV spike protein, or a pre-fusion MERS-CoV spike protein). The coronavirus vaccine may comprise a whole spike protein, a stabilized spike protein, a locked spike protein, a spike protein subunit, and/or a receptor-binding domain (RBD) from a spike protein. In some embodiments, the coronavirus vaccine comprises a SARS-CoV-2 spike protein (e.g., SARS-CoV-2-S1) or a subunit thereof. In some embodiments, the coronavirus vaccine comprises SARS-CoV-2-S1. In some embodiments, the coronavirus vaccine comprises SARS-CoV-2-S1fRSO9. In some embodiments, the coronavirus vaccine comprises MERS-S1. In some embodiments, the coronavirus vaccine comprises MERS-S1f. In some embodiments, the coronavirus vaccine comprises MERS-S1fRSO9. In some embodiments, the coronavirus vaccine comprises MERS-S1ffliC. In some embodiments, the coronavirus vaccine comprises a polypeptide comprising the amino acid sequence of SEQ ID NO: 1, or a functional fragment thereof.

The coronavirus vaccine may also comprise a gene product, vector, RNA, or DNA that encodes a spike protein or subunit thereof, and/or is configured to express a spike protein or subunit thereof, e.g., a whole spike protein, a stabilized spike protein, a locked spike protein, a spike protein subunit, or a receptor-binding domain (RBD) from a spike protein.

In some embodiments, the coronavirus vaccine comprises a nucleocapsid protein or subunit thereof, e.g., a SARS-CoV-2 nucleocapsid protein or subunit thereof, a SARS-CoV nucleocapsid protein or subunit thereof, and/or a MERS-CoV nucleocapsid protein or subunit thereof.

In some embodiments, the coronavirus vaccine comprises an inactivated coronavirus, e.g., inactivated SARS-CoV-2, inactivated SARS-CoV, and/or inactivated MERS-CoV. In some embodiments, the coronavirus vaccine comprises UV-inactivated coronavirus, e.g., UV-inactivated SARS-CoV-2, UV-inactivated SARS-CoV, and/or UV-inactivated MERS-CoV. In some embodiments, the coronavirus vaccine comprises inactivated SARS-CoV-2. In some embodiments, the coronavirus vaccine comprises UV-inactivated SARS-CoV-2. In some embodiments, the coronavirus vaccine comprises PiCoVacc.

The coronavirus vaccine may comprise an oligonucleotide such as DNA or RNA. In some embodiments, the coronavirus vaccine comprises DNA (e.g., a DNA plasmid. The DNA (e.g., DNA plasmid) may encode a coronavirus antigen (e.g., a coronavirus protein described herein), e.g., a SARS-CoV-2 protein, a SARS-CoV protein, and/or a MERS-CoV protein. In some embodiments, the coronavirus vaccine comprises DNA encoding a coronavirus spike protein or a subunit thereof (e.g., SARS-CoV-S1, SARS-CoV-2-S2, MERS-CoV-S1, or a subunit thereof). In some embodiments, the coronavirus vaccine comprises DNA encoding a SARS-CoV-2 spike protein. In some embodiments, the DNA (e.g., DNA plasmid) is configured to express a coronavirus antigen (e.g., a spike protein) in a subject, e.g., to induce an immune response. In some embodiments, the coronavirus vaccine comprises INO-4800.

In some embodiments, the coronavirus vaccine comprises RNA. The RNA may be messenger RNA (mRNA), self-amplifying mRNA (saRNA), nucleoside-modified mRNA (modRNA), or uridine-containing mRNA (uRNA). In some embodiments, the RNA is mRNA. In some embodiments, the mRNA is naked mRNA. The RNA (e.g., mRNA) may encode a coronavirus antigen (e.g., a coronavirus protein described herein), e.g., a SARS-CoV-2 protein, a SARS-CoV protein, and/or a MERS-CoV-2 protein. In some embodiments, the coronavirus vaccine comprises RNA encoding a coronavirus spike protein or a subunit thereof (e.g., SARS-CoV-2-S1, SARS-CoV-S1, MERS-CoV-S1, or a subunit thereof). In some embodiments, the coronavirus vaccine comprises mRNA encoding a SARS-CoV-2 spike protein or a subunit thereof. In some embodiments, the coronavirus vaccine comprises mRNA encoding a SARS-CoV spike protein or a subunit thereof. In some embodiments, the coronavirus vaccine comprises mRNA encoding a MERS-CoV spike protein or a subunit thereof. The RNA (e.g., mRNA) may be configured to express a coronavirus antigen (e.g., a spike protein) in a subject, e.g., to induce an immune response. In some embodiments, the coronavirus vaccine comprises e.g., mRNA-1273. In some embodiments, the coronavirus vaccine comprises an mRNA vaccine, such as the Pfizer-BioNTech and/or Moderna vaccines. In some embodiments, the coronavirus vaccine comprises a conventional inactivated vaccine, such as the BBIBP-CorV, CoronaVac, Covaxin, WIBP-CorV, CoviVac and/or QazVac vaccines. In some embodiments, the coronavirus vaccine comprises a viral vector vaccine, such as the Sputnik Light, Sputnik V, Oxford-AstraZeneca, Convidecia, and/or Johnson & Johnson vaccines. In some embodiments, the coronavirus vaccine comprises a protein subunit vaccine, such as the EpiVacCorona and/or RBD-Dimer vaccines.

In some embodiments, the coronavirus vaccine comprises a viral vector, e.g., a SARS-CoV-2 viral vector, a SARS-CoV viral vector, and/or a MERS-CoV viral vector. The viral vector may be a non-replicating viral vector or a replicating viral vector. In some embodiments, the coronavirus vaccine comprises an adenovirus vector, e.g., a replication-defective adenovirus vector. In some embodiments, the coronavirus vaccine comprises an adenovirus type 5 vector. In some embodiments, the adenovirus vector is configured to express a coronavirus antigen (e.g., a coronavirus protein, e.g., a coronavirus spike protein). In some embodiments, the adenovirus vector is configured to express a SARS-CoV-2 spike protein, or subunit thereof. In some embodiments, the coronavirus vaccine comprises Ad5-nCoV. In some embodiments, the coronavirus vaccine comprises Ad26-SARS-CoV-2.

In some embodiments, the coronavirus vaccine comprises a virus-like particle (VLP), e.g., a VLP comprising one or more coronavirus proteins. In some embodiments, the coronavirus vaccine comprises a SARS-CoV-2 VLP. In some embodiments, the coronavirus vaccine comprises a SARS-CoV VLP. In some embodiments, the coronavirus vaccine comprises a MERS-CoV VLP.

The coronavirus vaccine may also comprise a virus that is not a coronavirus, e.g., a virus that can act as a vector for a coronavirus antigen. For example, the coronavirus vaccine may comprise a live modified orthopoxvirus (e.g., horspepox) comprising a coronavirus antigen. In some embodiments, the coronavirus vaccine comprises a live virus. In some embodiments, the coronavirus comprises a live modified virus. In some embodiments, the coronavirus comprises a live modified orthopoxvirus, e.g., horsepox virus, comprising a coronavirus antigen. In some embodiments, the coronavirus comprises a live modified horsepox virus, comprising a SARS-CoV-2 antigen, a SARS-CoV antigen, and/or a MERS-CoV antigen. In some embodiments, the coronavirus comprises TNX-1800. In some embodiments, the coronavirus vaccine does not comprise a live virus.

In some embodiments, the coronavirus vaccine comprises a dendritic cell. A dendritic cell can be modified, e.g., with a lentiviral vector, to express a coronavirus gene product. In some embodiments, the coronavirus vaccine comprises a dendritic cell modified with a suitable vector (e.g., lentiviral vector) to express a coronavirus gene product, and/or is modified to comprise a coronavirus gene, e.g., a SARS-CoV-2 minigene, a SARS-CoV minigene, and/or a MERS-CoV minigene. In some embodiments, the coronavirus vaccine comprises LV-SMENP-DC. Similarly, the coronavirus vaccine may comprise an artificial antigen-presenting cell (aAPC). In some embodiments, an aAPCs is modified, e.g., with a suitable vector (e.g., lentiviral vector), to express a coronavirus gene product. In some embodiments, the coronavirus vaccine comprises an aAPC modified with a lentiviral vector to express a coronavirus gene product, e.g., a SARS-CoV minigene, a SARS-CoV-2 minigene, and/or a MERS-CoV minigene.

Influenza Vaccines

In another aspect, the present disclosure features a vaccine, a microneedle, and/or a microneedle device (e.g., a microneedle patch) comprising an influenza virus vaccine, antigen, and/or immunogen. The influenza virus is an RNA virus (e.g., a linear negative-sense single stranded RNA virus). There are four known genera of influenza virus, each containing a single type (e.g., influenza A, B, C, and D). Influenza viruses can continuously change and are subject to both antigenic drift and antigenic shift. Exemplary influenza strains are further described in WO 2019/195350, which is hereby incorporated by reference herein in its entirety.

Influenza A can be divided into subtypes on the basis of two proteins on the surface of the virus: hemagglutinin (HA) and neuraminidase (NA). Influenza A comprises 18 known HA subtypes, referred to herein as H1-H18, and 11 known NA subtypes, referred to herein as N1-N11. Many different combinations of HA and NA proteins may be found on the surface of the influenza A virus. For example, an “H1N1 virus” designates an influenza A virus subtype comprising an H1 protein and an N1 protein. Exemplary influenza A virus subtypes confirmed to infect humans include, but are not limited to, H1N1, H3N2, H2N2, H5N1, H7N7, H1N2, H9N2, H7N2, H7N3, H10N7, and H7N9. The H1N1 virus and H3N2 virus are currently in general circulation among humans.

Exemplary influenza B viruses may belong to, e.g., the B/Yamagata lineage (e.g., B/Phuket) and/or the B/Victoria lineage (e.g., B/Brisbane). In some embodiments, the influenza B viruses belongs to the B/Yamagata lineage. In some embodiments, the influenza B viruses belongs to the B/Phuket lineage. In some embodiments, the influenza B viruses belongs to the B/Victoria lineage. In some embodiments, the influenza B viruses belongs to the B/Brisbane lineage.

Non-limiting examples of influenza vaccines for use in the microneedles and microneedle devices (e.g., microneedle patches) described herein can include a commercial vaccine, such as a seasonal vaccine, a pandemic vaccine, and/or a universal vaccine; egg-based vaccines, cell-culture based vaccines; recombinant vaccines; live attenuated, inactivated whole virus, split virion, and/or protein subunit vaccines; and adjuvanted vaccines. In some embodiments, the microneedle device comprises egg-based vaccines. Various commercial influenza vaccines are listed below. Additionally, influenza vaccines comprising an HA stem antigen, RNA (e.g., mRNA), a DNA, a viral vector (e.g., adenovirus vector), and/or a virus-like particle (VLP) are suitable for use in the microneedles and microneedle devices (e.g., microneedle patches) described herein. In some embodiments, the influenza vaccine may target matrix protein 1, matrix protein 2 (M2e), and/or nucleoprotein (NP) of an influenza virus. In some embodiments, the vaccine is an egg-based vaccine. In some embodiments, the vaccine is grown in eggs, and later purified, inactivated, and/or split.

| | |
|---|---|
| Vaccine | Manufacturer |

Seasonal Influenza Vaccines

| | |
|---|---|
| Fluzone High Dose | Sanofi Pasteur |
| Fluzone Quadrivalent | Sanofi Pasteur |
| Fluzone Intradermal Quadrivalent | Sanofi Pasteur |
| Afluria/Fluvax | Seqirus |
| Agriflu | Seqirus |
| Fluad | Seqirus |
| Flucelvax | Seqirus |
| Fluvirin | Seqirus |
| Aggripal | Seqirus |
| FluMist Quadrivalent | MedImmune |
| Flublok | Protein Sciences (Sanofi Pasteur) |
| FluLaval | GlaxoSmithKline |
| Fluarix | GlaxoSmithKline |
| Influvac | Mylan |
| Preflucel | Nanotherapeutics |
| Anflu | Sinovac Biotech |

Pandemic Influenza Vaccines

| | |
|---|---|
| Influenza Virus Vaccine, H5N1 | Sanofi Pasteur |
| Pandemrix | GlaxoSmithKline |
| Panflu | Sinovac Biotech |
| Panflu 1 | Sinovac Biotech |

Vaccine Formulations

At least one vaccine, antigen, and/or immunogen described herein (e.g., at least one vaccine, antigen, and/or immunogen derived from an influenza virus described herein) can be incorporated into a variety of formulations, compositions, articles, devices, and/or preparations for administration, e.g., to achieve controlled- and/or sustained release. More particularly, at least one vaccine, antigen, and/or immunogen described herein (e.g., at least one vaccine, antigen, and/or immunogen derived from a coronavirus and/or an influenza virus described herein) can be formulated into formulations, compositions, articles, devices, and/or preparations by combination with appropriate, pharmaceutically acceptable carriers or diluents, and can be formulated into preparations in semi-solid, solid, or liquid formats. In some embodiments, the formulations, compositions, articles, devices, and/or preparations described herein comprise silk fibroin.

Exemplary formulations, compositions, articles, devices, and/or preparations comprise: a microneedle (e.g., a microneedle device, e.g., a microneedle patch, e.g., as described herein), an implantable device (e.g., a pump, e.g., a subcutaneous pump), an injectable formulation, a depot, a gel (e.g., a hydrogel), an implant, and a particle (e.g., a microparticle and/or a nanoparticle). As such, administration of the compositions can be achieved in various ways, including intradermal, intramuscular, transdermal, subcutaneous, or intravenous administration. Moreover, the formulations, compositions, articles, devices, and/or preparations can be formulated and/or administered to achieve controlled- and/or sustained release of the at least one vaccine, antigen, and/or immunogen described herein (e.g., at least one vaccine, antigen, and/or immunogen derived from a coronavirus and/or an influenza virus described herein).

In some embodiments, the vaccine (e.g., the coronavirus and/or the influenza vaccine) is administered, e.g., substantially sustained, over a period of, or at least 1, 5, 10, 15, 30, or 45 minutes; a period of, or at least, 1, 2, 3, 4, 5, 10, 24 hours; a period of, or at least, 1, 2, 3, 4, 5, 6, 7, 8, 9, 10, 11, 12, 13, 14 days; a period of, or at least, 1, 2, 3, 4, 5, 6, 7, 8 weeks; a period of, or at least 1, 2, 3, 4, 5, 6, 7, 8, 9, 10, 11 months; a period of, or at least, 1, 2, 3, 4, 5 years, or longer. In some embodiments, the vaccine (e.g., the coronavirus and/or the influenza vaccine) is administered, over a period of between about 4 and 30 days, e.g., between about 5 and 25 days, between about 10 and 20 days, between about 10 and 15 days, between about 12 and 18 days, between about 14 and 16 days, between about 14 and 15 days, e.g., about 4, 5, 6, 7, 8, 9, 10, 11, 12, 13, 14, 15, 16, 17, 18, 19, 20, 21, 22, 23, 24, 25 days.

In some embodiments, the vaccine (e.g., the coronavirus and/or influenza vaccine) is administered as a controlled- or sustained release formulation, dosage form, or device. In certain embodiments, the vaccine (e.g., the coronavirus and/or influenza vaccine) is formulated for continuous delivery, e.g., intradermal, intramuscular, and/or intravenous continuous delivery. In some embodiments, the composition or device for the controlled- or sustained-release of the vaccine is chosen from: a microneedle (e.g., a microneedle device, e.g., a microneedle patch), an implantable device (e.g., a pump, e.g., a subcutaneous pump), an injectable formulation, a depot, a gel (e.g., a hydrogel), an implant, or a particle (e.g., a microparticle and/or a nanoparticle). In one embodiment, the vaccine (e.g., the coronavirus and/or influenza vaccine) is in a silk-based controlled- or extended release dosage form or formulation (e.g., a microneedle described herein). In one embodiment, the vaccine (e.g., the coronavirus and/or influenza vaccine) is administered via an implantable device, e.g., a pump (e.g., a subcutaneous pump), an implant, an implantable tip of a microneedle, or a depot. The delivery method can be optimized such that a vaccine (e.g., a coronavirus vaccine and/or an influenza vaccine) dose as described herein (e.g., a standard dose) is administered and/or maintained in the subject for a pre-determined period (e.g., a period of, or at least: 1, 5, 10, 15, 30, 45 minutes; 1, 2, 3, 4, 5, 10, 24 hours; 1, 2, 3, 4, 5, 6, 7, 8, 9, 10, 11, 12, 13, 14 days; 1, 2, 3, 4, 5, 6, 7, 8 weeks; 1, 2, 3, 4, 5, 6, 7, 8, 9, 10, 11 months; 1, 2, 3, 4, 5 years, or longer). The substantially sustained or extended release of the vaccine (e.g., the coronavirus vaccine and/or influenza vaccine) can be used for prevention or treatment of a viral infection (e.g., a coronavirus infection and/or an influenza viral infection) for a period of hours, days, weeks, months, or years.

In some embodiments, the vaccine (e.g., the coronavirus and/or the influenza vaccine) is administered as a single-dose. In some embodiments, the vaccine (e.g., the coronavirus and/or the influenza vaccine) is administered as multiple doses (e.g., at least 2, 3, 4, 5 or more doses) at predetermined intervals. In some embodiments, a second or a subsequent dose of the vaccine may be administered at least about 1, 2, 3, 4, 5, 6, 7, 8, 9, 10, 11, 12, 13, 14 days; 1, 2, 3, 4, 5, 6, 7, 8 weeks; 1, 2, 3, 4, 5, 6, 7, 8, 9, 10, 11 months; 1, 2, 3, 4, 5 years, or longer) after a first or a previous dose. In some embodiments, the vaccine (e.g., the coronavirus and/or the influenza vaccine) may be administered annually. In some embodiments, the vaccine (e.g., the coronavirus and/or the influenza vaccine) may be administered as often as necessary to achieve immunity.

The present disclosure provides, in some embodiments, formulations, compositions, articles, devices, and/or preparations that can be formulated and/or configured for controlled- or sustained-release of at least one vaccine, antigen, and/or immunogen (e.g., at least one vaccine, antigen, and/or immunogen derived from a coronavirus and/or an influenza virus described herein) in an amount (e.g., a dosage) and/or over a time period sufficient to result in an immune response (e.g., a cellular immune response and/or a humoral immune response) to the virus, e.g., the coronavirus and/or the influenza virus, in the subject.

In some embodiments, the formulations, compositions, articles, devices, and/or preparations of the present disclosure can be formulated and/or configured for controlled- or sustained-release of at least one vaccine, antigen, and/or immunogen (e.g., at least one vaccine, antigen, and/or immunogen derived from a coronavirus and/or an influenza virus described herein) in an amount (e.g., a dosage) and/or over a time period sufficient to result in broad spectrum immunity in the subject.

The substantially continuously or extended release delivery or formulation of the vaccine (e.g., the coronavirus vaccine and/or the influenza vaccine) can be used for prevention or treatment of a viral infection (e.g., a coronavirus infection and/or an influenza viral infection) for a period of hours, days, weeks, months, or years.

In some embodiments, at least one vaccine, antigen, and/or immunogen described herein can be added to a silk fibroin solution, e.g., before forming silk fibroin microneedles or microneedle devices described herein. In embodiments, a silk fibroin solution can be mixed with a vaccine, antigen, and/or immunogen, and then used in the fabrication of an implantable microneedle tip, e.g., by the process of filling and/or casting, drying, and/or annealing to produce a microneedle having any of the desired material properties, as described herein.

Without being bound by theory, the ratio of silk fibroin to vaccine, antigen, and/or immunogen in an implantable tip of a microneedle influences their release. In some embodiments, increased silk concentration in the implantable tip favors a slower release and/or greater antigen retention within the tip. Any concentration of silk may be used, as long as the concentration allows for printing and has the mechanical strength sufficient to pierce the skin.

In some embodiments, silk fibroin can be used at a concentration ranging from about 1% w/v to about 10% w/v (e.g., about 1, 2, 3, 4, 5, 6, 7, 8, 9, or 10% w/v) in the fabrication of a microneedle, or a component thereof, as described herein.

Exemplary Excipients

The formulations, compositions, articles, devices (e.g., microneedle devices, e.g., microneedle patches), and/or preparations described herein, e.g., comprising vaccines, antigens, and/or immunogens, may be formulated with common excipients, diluents or carriers for administration by the intradermal, intramuscular, transdermal, subcutaneous, or intravenous routes. In some embodiments, the formulations, compositions, articles, devices, and/or preparations can be administered, e.g., transdermally, and can be formulated as controlled- or sustained-release dosage forms and the like. The formulations, compositions, articles, devices, and/or preparations described herein can be administered alone, in combination with each other, or they can be used in combination with other known therapeutic agents.

Suitable formulations for use in the present disclosure are found in Remington's Pharmaceutical Sciences (1985). Moreover, for a review of methods for drug delivery, see, Langer Science (1990) 249:1527-1533. The formulations, compositions, articles, devices, and/or preparations described herein can be manufactured in a manner that is known to those of skill in the art, e.g., by mixing, dissolving, granulating, dragee-making, levigating, emulsifying, encapsulating, entrapping or lyophilizing processes. The following methods and excipients are merely exemplary and are in no way limiting.

The silk fibroin formulations used in the fabrication of the microneedles described herein may include excipients. In embodiments, inclusion of an excipient may be for the purposes of improving the stability of an incorporated vaccine, antigen, and/or immunogen; to increase silk matrix porosity and diffusivity of the vaccine, antigen, and/or immunogen from the formulation, composition, article, device, preparation, and/or microneedle, e.g., microneedle tip; and/or to increase crystallinity/beta-sheet content of silk matrix to render the silk-material less soluble (e.g., insoluble).

Exemplary excipients include, but are not limited to, a sugar or a sugar alcohol (e.g., sucrose, trehalose, sorbitol, mannitol, or a combination thereof), a divalent cation (e.g., Ca²⁺, Mg²⁺, Mn²⁺, and Cu²⁺), a surfactant (e.g., a octyl phenol ethoxylate (e.g., Triton-X), a polysorbate, a poloxamer, and/or a polyethoxylated alcohol), polyol (e.g., glycerol), glycols (e.g. propylene glycol, PEG) and/or buffers. In some embodiments, the concentration of an excipient can be used to modify the porosity of the matrix, e.g., with sucrose being used as the most common excipient for this purpose. Excipients may also be added to favor silk self-assembly into order beta-sheet secondary structure, and such excipients generally can participate in hydrogen bonding or charge interactions with silk to achieve this effect. Non-limiting examples of excipients that can be used to favor silk self-assembly into order beta-sheet secondary structure include monosodium glutamate (e.g., L-glutamic acid), lysine, sugar alcohols (e.g., sorbitol and/or glycerol), and solvents (e.g., dimethylsulfoxide, methanol, and/or ethanol).

In some embodiments, the sugar or the sugar alcohol is sucrose present in an amount less than 70% (w/v), less than 60% (w/v), less than 50% (w/v), less than 40% (w/v), less than 30% (w/v), less than 20% (w/v), less than 10% (w/v), less than 9% (w/v), less than 8% (w/v), less than 7% (w/v), less than 6% (w/v), or 5% (w/v) or less, e.g., immediately before drying.

In some embodiments, the sugar or the sugar alcohol is sucrose present in an amount between about 1% (w/v) to about 10% (w/v), about 2% (w/v) to about 8% (w/v), about 2.2% (w/v) to about 6% (w/v), about 2.4% (w/v) to about 5.5% (w/v), about 2.5% to about 5%, or about 2.4% (w/v), about 2.5%, or about 5% (w/v), e.g., immediately before drying.

In some embodiments, the sugar or the sugar alcohol is trehalose present in an amount between about 1% (w/v) to about 10% (w/v), about 2% (w/v) to about 8% (w/v), about 2.2% (w/v) to about 6% (w/v), about 2.4% (w/v) to about 5.5% (w/v), about 2.5% to about 5%, or about 2.4% (w/v), about 2.5%, or about 5% (w/v), e.g., immediately before drying.

In some embodiments, the sugar or the sugar alcohol is sorbitol present in an amount between about 1% (w/v) to about 10% (w/v), about 2% (w/v) to about 8% (w/v), about 2.2% (w/v) to about 6% (w/v), about 2.4% (w/v) to about 5.5% (w/v), about 2.5% to about 5%, or about 2.4% (w/v), about 2.5%, or about 5% (w/v), e.g., immediately before drying.

In some embodiments, the sugar or the sugar alcohol is glycerol present in an amount between about 1% (w/v) to about 10% (w/v), about 2% (w/v) to about 8% (w/v), about 2.2% (w/v) to about 6% (w/v), about 2.4% (w/v) to about 5.5% (w/v), about 2.5% to about 5%, or about 2.4% (w/v), about 2.5%, or about 5% (w/v), e.g., immediately before drying.

In some embodiments, the surfactants (e.g., a octyl phenol ethoxylate (e.g., Triton-X), a polysorbate, a poloxamers, and/or a polyethoxylated alcohol) is present in an amount between about 0.005% (w/v) to about 1% (w/v), about 1% (w/v) to about 10% (w/v), about 2% (w/v) to about 8% (w/v), about 2.2% (w/v) to about 6% (w/v), about 2.4% (w/v) to about 5.5% (w/v), about 2.5 to about 5%, or about 2.4% (w/v), about 2.5%, or about 5% (w/v), e.g., immediately before drying.

In some embodiments, the polyol (e.g., glycerol) is present in an amount between about 1% (w/v) to about 10% (w/v), about 2% (w/v) to about 8% (w/v), about 2.2% (w/v) to about 6% (w/v), about 2.4% (w/v) to about 5.5% (w/v), about 2.5 to about 5%, or about 2.4% (w/v), about 2.5%, or about 5% (w/v), e.g., immediately before drying.

In some embodiments, the glycols (e.g. propylene glycol, e.g., PEG) is present in an amount between about 1% (w/v) to about 10% (w/v), about 2% (w/v) to about 8% (w/v), about 2.2% (w/v) to about 6% (w/v), about 2.4% (w/v) to about 5.5% (w/v), about 2.5 to about 5%, or about 2.4% (w/v), about 2.5%, or about 5% (w/v), e.g., immediately before drying.

In some embodiments, the vaccine preparation further comprises a divalent cation. In some embodiments, the divalent cation is selected from the group consisting of Ca²⁺, Mg²⁺, Mn²⁺, and Cu²⁺. In some embodiments, the divalent cation is present in the preparation, e.g., immediately before drying, in an amount between 0.1 mM and 100 mM. In some embodiments, the divalent cation is present in the preparation, e.g., immediately before drying, in an amount between 10⁻⁷ and 10⁻⁴ moles per standard dose of viral immunogen. In some embodiments, the divalent cation is present in the preparation immediately before drying in an amount between 10⁻¹⁰ to 2×10⁻³ moles.

In some embodiments, the vaccine preparation further comprises poly(lactic-co-glycolic acid) (PGLA).

In some embodiments, the vaccine, antigen, and/or immunogen preparation further comprises a buffer, e.g., immediately before drying. In some embodiments, the buffer has buffering capacity between pH 3 and pH 8, between pH 4 and pH 7.5, or between pH 5 and pH 7. In some embodiments, the buffer is selected from the group consisting of 4-(2-hydroxyethyl)-1-piperazineethanesulfonic acid (HEPES) buffer and a citrate-phosphate (CP) buffer. In some embodiments, the buffer is present in the preparation, e.g., immediately before drying, in an amount between 0.1 mM and 100 mM. In some embodiments, the buffer is present in an amount between 10⁻⁷ and 10⁻⁴ moles per standard dose of viral immunogen. In some embodiments, the buffer is present in an amount between 10⁻¹⁰ to 2×10⁻³ moles.

In addition, the vaccine, antigen, and/or immunogen can also be formulated as a depot, gel, or hydrogel preparation. Such long acting formulations can be administered by implantation (for example subcutaneously or intramuscularly) or by intramuscular injection. Thus, for example, the vaccine can be formulated with suitable polymeric or hydrophobic materials (for example as an emulsion in an acceptable oil) or ion exchange resins, or as sparingly soluble derivatives, for example, as a sparingly soluble salt.

In one embodiment, the vaccine, antigen, and/or immunogen is administered via an implantable infusion device, e.g., a pump (e.g., a subcutaneous pump), an implant or a depot. Implantable infusion devices typically include a housing containing a liquid reservoir which can be filled transcutaneously by a hypodermic needle penetrating a fill port septum. The medication reservoir is generally coupled via an internal flow path to a device outlet port for delivering the liquid through a catheter to a patient body site. Typical infusion devices also include a controller and a fluid transfer mechanism, such as a pump or a valve, for moving the liquid from the reservoir through the internal flow path to the device's outlet port.

In some embodiments, the vaccine, antigen, and/or immunogen can be packaged and/or formulated as a particle, e.g., a microparticle and/or a nanoparticle. Typically nanoparticles are from 10, 15, 20, 25, 30, 35, 45, 50, 75, 100, 150 or 200 nm or 200-1,000 nm in diameter, e.g., 10, 15, 20, 25, 30, 35, 45, 50, 75, 100, 150, or 200, or 20 or 30 or 50-400 nm in diameter. Smaller particles tend to be cleared more rapidly form the system. Therapeutic agents, including vaccines, can be entrapped within or coupled, e.g., covalent coupled, or otherwise adhered, to nanoparticles.

Lipid- or oil-based nanoparticles, such as liposomes and solid lipid nanoparticles (LNPs) and can be used to can be used to deliver vaccines, antigens, and/or immunogens, optionally with an additional therapeutic agent, described herein. Solid LNPs for the delivery of therapeutic agents are described in Serpe et al. Eur. J. Pharm. Bioparm. (2004) 58:673-680 and Lu et al. Eur. J. Pharm. Sci. (2006) 28: 86-95. Polymer-based nanoparticles, e.g., PLGA-based nanoparticles can be used to deliver agents described herein. These tend to rely on biodegradable backbone with the therapeutic agent intercalated (with or without covalent linkage to the polymer) in a matrix of polymer. PLGA is a widely used in polymeric nanoparticles, see Hu et al. J. Control. Release (2009) 134:55-61; Cheng et al. Biomaterials (2007) 28:869-876, and Chan et al. Biomaterials (2009) 30:1627-1634. PEGylated PLGA-based nanoparticles can also be used to deliver therapeutic agents, see, e.g., Danhhier et al., J. Control. Release (2009) 133:11-17, Gryparis et al Eur. J. Pharm. Biopharm. (2007) 67:1-8. Metal-based, e.g., gold-based nanoparticles can also be used to deliver therapeutic agents. Protein-based, e.g., albumin-based nanoparticles, can be used to deliver agents described herein. In some embodiments, a therapeutic agent can be bound to nanoparticles of human albumin.

In some embodiments, the vaccine, antigen, and/or immunogen is encapsulated by an LNP, and/or formulated as a lipid nanoparticle (LNP) formulation. In some embodiments, the lipid nanoparticle comprises one or more lipids such as an ionizable lipid (e.g., SM-102), cholesterol, a phospholipid (e.g., 1,2-distearoyl-sn-glycero-3-phosphocholine (DSPC)), and/or a PEG-containing lipid (e.g., 1,2-dimyristoyl-rac-glycero-3-methoxypolyethylene glycol-2000 (PEG2000 DMG).

A broad range of nanoparticles are known in the art. Exemplary approaches include those described in WO2010/005726, WO2010/005723 WO2010/005721, WO2010/121949, WO2010/0075072, WO2010/068866, WO2010/005740, WO2006/014626; and U.S. Pat. Nos. 7,820,788 and 7,780,984, the contents of which are incorporated herein by reference in their entirety.

Dosages

Any dosage amount (e.g., a standard dose and/or a fractional dose) of a vaccine, antigen, and/or immunogen that is capable of eliciting an immune response (e.g., immunogenicity and/or broad-spectrum immunity) in a subject, e.g., when administered by a microneedle of the present disclosure, may be used according to the methods described herein.

In some embodiments, the dose, e.g., the standard dose (e.g., human dose) for a vaccine, an antigen, and/or an immunogen (e.g., a coronavirus vaccine and/or an influenza vaccine) is between about 0.1 μg and about 1000 μg (e.g., between about 0.1 μg and about 750 μg, between about 0.1 μg and about 500 μg, between about 0.1 μg and about 250 μg, between about 0.1 μg and about 200 μg, between about 0.1 μg and about 150 μg, between about 0.1 μg and about 125 μg, between about 0.1 μg and about 100 μg, between about 0.1 μg and about 75 μg, between about 0.1 μg and about 65 μg, between about 0.1 μg and about 50 μg, between about 0.1 μg and about 40 μg, between about 0.1 μg and about 30 μg, between about 0.1 μg and about 20 μg, between about 0.1 μg and about 10 μg, between about 0.1 μg and about 1 μg, between about 0.5 μg and about 5 μg, between about 5 μg and about 10 μg, between about 10 μg and about 20 μg, between about 20 μg and about 30 μg, between about 30 μg and about 40 μg, about 40 μg and about 50 μg, about 50 μg and about 65 μg, e.g., about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 100, 125, 150, 175, 200, 225, 250, 300, 350, 400, 450, 500, 550, 600, 650, 700, 750, 800, 850, 900, 950, or 1000 μg).

In some embodiments, the dose, e.g., the standard dose (e.g., human dose) for a vaccine, antigen, and/or immunogen (e.g., a coronavirus vaccine) is between about 0.1 μg and about 500 μg, (e.g., between about 0.1 μg and about 500 μg, between about 0.1 μg and about 400 μg, between about 0.1 μg and about 300 μg, between about 0.1 μg and about 200 μg, between about 0.1 μg and about 100 μg, between about 1 μg and about 500 μg, between about 10 μg and about 500 μg, between about 25 μg and about 500 μg, between about 50 μg and about 500 μg, or between about 100 μg and about 500 μg, e.g., about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 75, 100, 125, 150, 175, 200, 225, 250, 275, or 300 μg.

In some embodiments, the dose, e.g., the standard dose (e.g., human dose) for a vaccine, an antigen, and/or an immunogen (e.g., an influenza vaccine) is between about 0.1 μg and about 65 μg (e.g., between about 0.1 μg and about 10 μg, between about 0.1 μg and about 1 μg, between about 0.5 μg and about 5 μg, between about 5 μg and about 10 μg, between about 10 μg and about 20 μg, between about 20 μg and about 30 μg, between about 30 μg and about 40 μg, about 40 μg and about 50 μg, about 50 μg and about 65 μg, e.g., about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, or 65 μg). In some embodiments, the dose, e.g., standard human dose, for a vaccine described herein (e.g., an influenza vaccine) is approximately between about 1 μg and about 30 μg per strain, e.g., between about 5 μg and about 30 μg per strain of the virus (e.g., about 5, 6, 7, 8, 9, 10, 11, 12, 13, 14, 15, 16, 17, 18, 19, 20, 21, 22, 23, 24, 25, 26, 27, 28, 29, or 30 μg per strain).

In some embodiments, the dose, e.g., fractional dose, for a vaccine described herein (e.g., a coronavirus vaccine and/or an influenza vaccine) is no more than 1/X, wherein X is any number, e.g., wherein X is 0.1, 0.2, 0.3, 0.4, 0.5, 0.6, 0.7, 0.8, 0.9, 1, 2, 3, 4, 5, 6, 7, 8, 9, 10, 15, 20, 30, 40, 50, 60, 70, 80, 90, or 100 or more, of the total dose (e.g., a standard dose). Due in part to relatively high populations of antigen-presenting cells in the skin, dose-sparing may be possible when delivering vaccines to the intradermal space, e.g., when delivering a coronavirus antigen and/or an influenza antigen to the intradermal space. Accordingly, in some embodiments the total dosage amount of a vaccine, e.g., a coronavirus vaccine and/or influenza vaccine that can be delivered by a microneedle of the present disclosure can be between about 5 μg and 13 μg (e.g., about 5 μg, about 6 μg, about 7 μg, about 8 μg, about 9 μg, about 10 μg, about 11 μg, about 12 μg, or about 13 μg).

Without wishing to be bound by theory, the total dosage amount (e.g., a standard dose) of a vaccine, antigen, and/or immunogen to be administered by a microneedle described herein can be divided between a plurality of microneedles (e.g., within a patch), such that a microneedle tip can comprises a portion of the total dosage amount. For example, in an array comprising about 121 microneedles, one or more microneedles (e.g., each microneedle) may comprise at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25% or more of the total dosage amount of a coronavirus vaccine and/or an influenza vaccine. In some embodiments, an implantable microneedle tip, as described herein, can comprise about 0.1 μg to about 65 μg (e.g., about 0.1 μg, about 0.2 μg, about 0.3 μg, about 0.4 μg, about 0.5 μg, about 0.6 μg, about 0.7 μg, about 0.8 μg, about 0.9 μg, about 1 μg, about 1 μg to about 10 μg, about 10 μg to about 20 μg, about 20 μg to about 30 μg, about 30 μg to about 40 μg, about 40 μg to about 50 μg, about 50 μg to about 65 μg) of a vaccine, antigen, and/or immunogen, as described herein. In some embodiments one or more microneedles (e.g., each microneedle) of a microneedle device described herein may comprises between about 0.002 μg and about 5 μg of the vaccine (e.g., coronavirus vaccine and/or influenza vaccine), e.g., at least about 0.003 μg, 0.004 μg, 0.005 μg, 0.01 μg, 0.02 μg, 0.03 μg, 0.04 μg, 0.05 μg, 0.06 μg, 0.07 μg, 0.08 μg, 0.09 μg, 0.1 μg, 0.12 μg, 0.14 μg, 0.16 μg, 0.18 μg, 0.2 μg, 0.25 μg, 0.3 μg, 0.35 μg, 0.4 μg, 0.45 μg, 0.5 μg, 0.6 μg, 0.7 μg, 0.8 μg, 0.9 μg, 1.0 μg, 1.2 μg, 1.4 μg, 1.6 μg, 1.8 μg, 2.0 μg, 2.5 μg, 3.0 μg, 3.5 μg, 4.0 μg, 4.5 μg, or 5 μg of the vaccine.

In some embodiments, the vaccine dosage amount loaded into a microneedle patch can be manipulated via the concentration of antigen in the formulated solution that forms the needle tips, the volume of solution dispensed into each needle tip, and the total number of needles (the former two are generally more convenient means of varying dose). The dosage released into the skin is related to deployment efficiency (the portion of needle tips that are left behind in the skin after the patch is removed), and also the release profile over time and the residence time of the tips within the skin. Because of the continuous sloughing of skin from the epidermis, deeper deployment within the skin can be related to longer residence time. Therefore, it is desirable to maximize the penetration depth of the needle tip (up to a limit defined by the depth of pain receptors within the skin, e.g., at a depth of between about 100 μm and about 600 μm), and also to have the antigen spatially concentrated toward the tip of the needle.

The formulations, compositions, articles, devices, and/or preparations described herein, including the implantable sustained-release tip formulation, are designed to not only sustain release of vaccine antigen over the duration, e.g., of tip retention in the dermis, but to also maintain stability of antigen during this period of time (e.g., at least about 1-2 weeks). In some embodiments, approximately 95-100% of the total dosage amount incorporated, e.g., in a formulation, composition, article, device, preparation, and/or microneedle described herein, can be expected to be available for delivery, e.g., into a subject, e.g., into a tissue of a subject, such as the skin, a mucous membrane, an organ tissue, a buccal cavity, a tissue, or a cell membrane. Without being bound by theory, successful deployment of a microneedle into the skin is at least about 50% and can be as high as 100% of an array (e.g., upon application at least about 50%, 60%, 70%, 80%, 90% or more (e.g., 100%) of the total number of microneedle comprising an array are successfully deployed within, e.g., the skin, for controlled- or sustained-release of a vaccine antigen). In some embodiments, a portion of antigen may not be released from the silk tips during the duration of deployment.

Exemplary Adjuvants and Therapeutic Agents

The microneedles and microneedle devices (e.g., microneedle patches) described herein may be configured to administer one or more vaccine with an additional therapeutic agent and/or adjuvant. In some embodiments, an additional therapeutic agent and/or adjuvant agent may be formulated in the same tip as a vaccine. For example, a vaccine, antigen, and/or immunogen may be co-delivered, e.g., by a microneedle device, with one or more adjuvant. Without wishing to be bound by theory, such a combination could drive stronger cellular immune responses and/or mucosal responses.

Adjuvants may be used to favor or amplify the cascade of immunological events, ultimately leading to an increased immunological response, e.g., the integrated bodily response to an antigen, including cellular and/or humoral immune responses. Non-limiting examples of adjuvants that may be combined with a vaccine described herein, e.g., a coronavirus vaccine and/or an influenza vaccine, include: aluminum (e.g., aluminum gels and/or aluminum salts, such as aluminum hydroxide, aluminum phosphate, and aluminum potassium sulfate), lipids (e.g., squalene, monophosphoryl lipid A (MPL)), AS03 (e.g., an adjuvant comprising D,L-alpha-tocopherol (vitamin E), squalene, and polysorbate 80), squalene-based adjuvants (e.g., MF59®), cytosine phosphoguanine-based adjuvants (e.g., CpG 1018), adjuvants derived from delta inulin (e.g., Advax adjuvant), AS04 (e.g., an adjuvant comprising a combination of aluminum hydroxide and MPL) AS01 (e.g., a liposome-based adjuvant comprising a combination of 3-O-desacyl-4′-monophosphoryl lipid A (MPL) and a saponin, such as QS-21), immunostimulating complex (ISCOM) (e.g., an adjuvant comprising a combination of saponin and phospholipid), and saponin-based adjuvants (e.g. QS21 or Matrix M).

In some embodiments, a microneedle or microneedle device (e.g., microneedle patch) does not contain an adjuvant. For example, without wishing to be bound by theory, the sustained antigen presentation provided by the microneedle devices (e.g., microneedle patches) described herein may eliminate the need for adjuvants. Accordingly, a microneedle or microneedle device (e.g., microneedle patch) may be utilized to enhance, accelerate, and/or broaden immune responses and/or to reduce the number of doses required (“dose sparing”), which can be an important advantage over conventional vaccine formulations given the costs of producing vaccine antigens and the challenges of multiple clinic visits when vaccine boosting is required to achieve protective immune responses.

In some embodiments, a microneedle or microneedle device (e.g., microneedle patch) described herein can be fabricated to administer at least one additional therapeutic agent. Various forms of a therapeutic agent can be used which are capable of being released from the microneedles described herein into adjacent tissues or fluids upon administration to a subject. In some embodiments, an additional therapeutic agent can be included within the base layer and/or within the implantable tip.

Examples of additional therapeutic agents that can be used according to the methods of the present disclosure, e.g., incorporated into a microneedle described herein, e.g., during fabrication, include steroids and esters of steroids (e.g., estrogen, progesterone, testosterone, androsterone, cholesterol, norethindrone, digoxigenin, cholic acid, deoxycholic acid, and chenodeoxycholic acid), boron-containing compounds (e.g., carborane), chemotherapeutic nucleotides, drugs (e.g., antibiotics, antivirals, antifungals), enediynes (e.g., calicheamicins, esperamycins, dynemicin, neocarzinostatin chromophore, and kedarcidin chromophore), heavy metal complexes (e.g., cisplatin), hormone antagonists (e.g., tamoxifen), non-specific (non-antibody) proteins (e.g., sugar oligomers), oligonucleotides (e.g., mRNA sequences or antisense oligonucleotides that bind to a target nucleic acid sequence), peptides, proteins, antibodies, photodynamic agents (e.g., rhodamine 123), radionuclides (e.g., I-131, Re-186, Re-188, Y-90, Bi-212, At-211, Sr-89, Ho-166, Sm-153, Cu-67 and Cu-64), toxins (e.g., ricin), and transcription-based pharmaceuticals.

In some embodiments a microneedle or microneedle device described herein may comprise, and/or be configured to release, a non-vaccine molecule, e.g., a molecule useful to confirm dose delivery. The inclusion of a non-vaccine molecule (e.g., a dye) in a microneedle or microneedle device may be used to confirm localization within the microneedle or microneedle device (e.g., in the microneedle tip), and/or used to confirm release in a subject, e.g., to confirm dose delivery. In some embodiments, the vaccine, antigen, and/or immunogen described herein (e.g., a coronavirus vaccine and/or influenza vaccine) is co-formulated with a non-vaccine molecule. The non-vaccine molecule, e.g., useful for confirming dose delivery, may be any suitable dye molecule, e.g., a biocompatible dye molecule, or a reporter molecule. In some embodiments, the non-vaccine molecule can be visualized, e.g., by illuminating under UV irradiation, after administration to a subject. In some embodiments, the non-vaccine molecule is a dye. In some embodiments, the non-vaccine molecule is a biocompatible dye. In some embodiments, the non-vaccine molecule can be illuminated under UV irradiation, e.g., to facilitate visualization, e.g., in a microneedle and/or in a subject.

### Methods of Fabricating a Microneedle and Microneedle Device

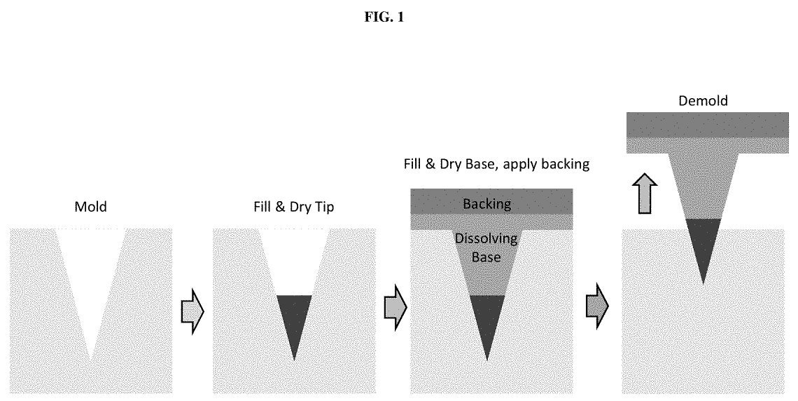

Described herein are methods of fabricating a microneedle or microneedle device, e.g., as described herein. A schematic diagram depicting the method of fabrication of a microneedle of the present disclosure is shown in FIG. 1. Machine vision guided dispensing of precise nL volumes of a solution, e.g., a silk fibroin solution comprising a vaccine, antigen, and/or immunogen, into individual needle cavities enables different dosages and formulations to be incorporated within releasable tips of a microneedle device (e.g., a microneedle array or patch).

In some embodiments, one or more therapeutic agents, such as one or more vaccines, antigens, and/or immunogens may be printed into the same microneedle or into different microneedles during the fabrication of a microneedle device (e.g., a microneedle array or patch) described herein.

In some embodiments, one or more coronavirus vaccines and/or one or more influenza vaccines may be printed into the same microneedle or into different microneedles during the fabrication of a microneedle device (e.g., a microneedle array or patch) described herein.

In some embodiments, one or more coronavirus vaccines may be printed into the same microneedle or into different microneedles during the fabrication of a microneedle device (e.g., a microneedle array or patch) described herein.

In some embodiments, one or more influenza vaccines may be printed into the same microneedle or into different microneedles during the fabrication of a microneedle device (e.g., a microneedle array or patch) described herein.

In certain embodiments, a coronavirus vaccine and an influenza vaccine are in the same microneedle. In certain embodiments, a coronavirus vaccine and an influenza vaccine are in different (e.g., separate) microneedles.

In some embodiments, one or more mRNA molecules, such as one or more mRNA vaccines, may be printed into the same microneedle or into different microneedles during the fabrication of a microneedle device (e.g., a microneedle array or patch) described herein.

An exemplary microneedle device (e.g., a microneedle array or patch), comprises an 11×11 cone array. It should be understood that the microneedle device may include needle cavities produced in an array of varying number of cavities and orientations to achieve a desired result.

Mold Production

In some embodiments, a mold is used in the fabrication of a microneedle device. As will be discussed in greater detail below, a sterilized mold may be used to produce a microneedle device having an array of releasable tips embodying an antigen formulation, e.g., an antigen-silk formulation, such as a formulation comprising a coronavirus antigen, an influenza antigen, or a combination or vaccine preparation thereof.

For example, a silicone (DOW Corning Sylgard® 184) resin may be cast against a positive master having the intended geometry of a microneedle array. Once the silicone has cured, it may be removed from the master. The master can then be reused for a large number of silicone castings. Throughout the fabrication process the silicone mold may be inspected for defects (e.g., between castings). If desired, the silicone mold can be sterilized, for example, by autoclaving. In one embodiment, the mold includes a mold body having an array of needle cavities formed within the mold body.

In some embodiments, other types of silicone and/or other materials and processes may be used to fabricate the mold. For example, liquid silicone injection molding and thermoplastic elastomer injection molding may be used. Without wishing to be bound by theory, it may be understood that a key requirement is that the mold material be soft and flexible (e.g., comprise a Shore hardness of about 50A) and have low adhesion with silk and other materials used in the construction of the patch.

Tip Filling

The fabrication of a microneedle device described herein may involve a step of tip filling, such as filling a mold with a tip formulation. For example, a tip formulation comprising a silk fibroin, and/or another suitable formulation for a microneedle tip (e.g., a formulation described herein); a vaccine, antigen, and/or immunogen; and/or other excipients, adjuvants, and/or non-vaccine molecules (e.g., dyes) in aqueous solution, is dispensed into each needle cavity in the mold via nanoliter printing. This may be achieved at lab scale using machine vision guided automated dispensing system, such as a Biojet Elite™ AD3400 dispensing system produced by BioDot, but systems with similar capabilities made by other suppliers can be employed. The working volume of the BioDot™ dispenser can be enclosed and maintained at about 60% relative humidity (RH) to slow drying of the formulation and avoid buildup of dry solids on the dispensing nozzle.

Machine vision guided printing of precise volumes (e.g., nanoliter volumes) of tip formulations (e.g., a silk fibroin solution, e.g., an antigen-silk fibroin solution) can provide microneedle devices comprising different dosages and/or formulations within the tips of the microneedles. In some embodiments, precision filling of each individual microneedle tip can enable different patterns of vaccine delivery, dosing schemes, different combinations of antigens, and/or microneedles comprising different combinations of antigens, therapeutic agents, and/or adjuvants. In some embodiments, where co-formulation is not possible, the manufacturing process can be adapted in order to dispense a first tip formulation into a portion of the needle array and then dispense a second formulation into a different portion of the needle array. An enlarged view of a portion of a microneedle device of the present disclosure, fabricated to include different formulations in different microneedles, is shown in FIG. 5.

Using these methods, a microneedle device can be prepared comprising, for example, five different antigens each independently formulated into different microneedles. In some embodiments, tip filling comprises independently filling microneedle cavities with a coronavirus antigen, or one of four different influenza antigens, such that the device comprises all of the coronavirus antigen and four influenza antigens individually formulated into different microneedles. In some embodiments, tip filling comprises filling one or more microneedle cavities with a coronavirus antigen, and filling the remaining microneedle cavities with a co-formulation of influenza antigens. In some embodiments, tip filling comprises filling each of the microneedle cavities with a co-formulation of a coronavirus antigen and one or more influenza antigens (e.g., four influenza antigens).

Molds can be placed within a fixture that constrains their locations on the processing platform of the BioDot™ dispenser. The machine uses a camera to image each mold and a machine vision algorithm identifies the precise location and orientation of the array of needle cavities in each mold. This location can be used to direct the subsequent dispensing steps. The filled molds can be inspected using a stereomicroscope for filling defects such as misaligned dispenses or large bubbles in the liquid, however any other suitable method or instrument for inspection of the filled molds may be employed.

Primary Drying

After filling the molds with a tip formulation, the filled molds may undergo a primary drying step. For example, the filled molds can be set aside to dry within the machine enclosure for about 7 minutes. After drying, the above dispensing process can be repeated. In some embodiments, the molds are moved to a chamber with approximately saturated humidity and incubated overnight to slowly dry the tips. During this time the silk structure shifts to more beta-sheets and can become less soluble or insoluble (annealing). As described herein, and without wishing to be bound by theory, the process of annealing, e.g., to alter the beta-sheet content, may be used to fine tune the solubility of the silk tip matrix to alter the ability of vaccine, antigen, and/or immunogen to be retained (e.g., to provide controlled or sustained release from the microneedle tip), and/or to increase the mechanical strength of the microneedle tip.

Secondary Drying

In some embodiments, the molds are moved to a chamber in which humidity is controlled to about 10% to about 25% relative humidity at room temperature (e.g., about 25° C.), and kept overnight (about 14 hours) to complete drying. This is the “secondary” drying step.

Water Annealing

The method of fabricating microneedles or microneedle devices can employ a step of water annealing. In some embodiments, the molds are transferred to a vacuum desiccator that also contains about 500 mL of deionized water. The desiccator can then be closed and placed under vacuum for about 5 minutes, e.g., using the main vacuum line in the lab. After 5 minutes, the outlet valve of the desiccator can be closed and the desiccator placed within an incubator holding at about 37° C. for about four hours. After four hours, the desiccator can be vented and the molds transferred back to the 25% relative humidity chamber at ambient room temperature (e.g., about 25° C.).

Post-Anneal Drying

In some embodiments, a method of fabricating microneedles involves a step of post-anneal drying. For example, molds can be kept at about 10% to about 25% relative humidity for at least four hours or up to overnight before subsequent steps.

Base Layer Filling

The base layer (e.g., dissolvable base layer) may be formed by filling the mold with a base solution described herein. In some embodiment, the base solution is 40% w/v hydrolyzed gelatin and 10% w/v sucrose in deionized water. In some embodiments, the base solution is 30% dextran 70 kDa, 10% sucrose, 1% glycerol, and 0.01% Triton-X. In some embodiments, 150 μL of base solution is spread evenly over the mold, e.g., using a pipette. Next, the molds can be centrifuged at 3900 rpm for up to about 2 minutes. In some embodiments, the molds are inspected, and if any needle cavities remain unfilled, the filling and centrifuging process can be repeated. The molds can then be “topped off” with 50 μL of base solution. In some embodiments, centrifuge filling can be used. In some embodiments, the base is filled in the same manner as the tips by use of a vision-guided droplet dispensing into the mold cavities.

Base Drying

After filling the base layer, the base layer may then be dried. For example, the filled molds can be transferred back to the chamber at about 10% to about 25% relative humidity, and dried at least overnight and up to 3 days.

Backing Application

The microneedle devices (e.g., microneedle patches) described herein, e.g., used for the release, e.g., controlled- or sustained-release of a vaccine, antigen, and/or immunogen, and to provide improved immunogenicity (see, e.g., the Examples) may comprise a paper backing layer. In some embodiments, the microneedle devices (e.g., microneedle patches) comprise an adhesive backing, that may or may not further comprise a solid support. In some embodiments, the microneedle devices (e.g., microneedle patches) comprise adhesive plastic tape backing layers. In some embodiments, adhesive plastic tape has superior performance as a backing layer, e.g., compared to paper backing layers.

In some embodiments, the paper backing process is as follows: the dried base layer is partially re-wetted with 10-30 μL of deionized water spread over the surface with a pipette. Whatman 903 paper is punched into 12 mm diameter circles. The circles of paper are gently pressed into the wet surface of the base layer. The wet base layer partially soaks into the paper. The molds with backing are transferred back into the 25% relative humidity chamber to dry for at least 4 hours, or until ready for use.

In some embodiments, the adhesive tape backing process is as follows: adhesive-backed polyester tape (e.g., 3M® Magic™ tape) is cut into a piece about 12 mm wide and about 25 mm long. One end of the tape is aligned with the patch and gently pressed onto the surface of the base layer. The free end of the tape is folder over onto itself to form a non-adhesive “handle.”

Demolding

The microneedle devices (e.g., microneedle patches) are removed from the mold before use. The flexible mold is gently bent away from the stiffer patch, and the patch is taken away from the mold. The patch can be inspected for defects such as missing or broken needles.

Packaging

The microneedle devices (e.g., microneedle patches) may be used soon after demolding, for example as in the studies above, and not require packaging. If extended storage is needed, assembled patches can be packaged in a container with low moisture vapor transmission rate (e.g., a glass vial or thermoformed plastic tray made of low moisture vapor transmission rate (MVTR) materials and a foil-backed heat-sealed lid) along with a desiccant to maintain between about 0% and about 50% (e.g., between about 0% and 10%, between about 10% and about 20%, between about 20% and about 30%, between about 30% and about 40%, or between about 40% and 50%, e.g., about 25%) relative humidity inside the package.

Methods and Compositions for the Stabilization, Storage, and Delivery of Messenger RNA (mRNA)

Provided herein are various methods and compositions for stabilizing, storing, and delivering of messenger RNA (mRNA) based therapeutics. Without being bound by theory, the successful use of messenger RNA (mRNA) as a therapeutic modality is impeded by numerous factors, including, but not limited to, challenges associated with an mRNA molecule's relatively large size, intrinsic instability, and tendency to degrade. Current techniques to stabilize mRNA therapeutics often involve extreme cold conditions, which often is impractical and expensive. Accordingly, the storage and delivery of mRNA based therapeutics represents a major hurdle. There exists a need for improved methods and compositions for the storage and delivery of mRNA-based therapeutics.

The present disclosure is based, at least in part, on the surprising discovery that the formulations, compositions, articles, microneedle and microneedle devices, and/or preparations described herein, including the implantable sustained-release tip formulations, can maintain the stability of a therapeutic agent, such as an mRNA, at various environmental conditions including, but not limited to temperature, pH, and humidity. Accordingly, in some embodiments, the formulations, compositions, articles, microneedle and microneedle devices, and/or preparations described herein, including the implantable sustained-release tip formulations, can improve the storage stability of inherently unstable mRNA therapeutics, including mRNA vaccines, by preventing and/or reducing degradation of an mRNA during prolonged storage.

In certain embodiments, the formulations, compositions, articles, microneedle and microneedle devices, and/or preparations described herein, including the implantable sustained-release tip formulations, stabilize a therapeutic agent, such as an mRNA, during prolonged storage at various environmental conditions including, but not limited to, temperatures at or above 4° C.

In some embodiments, the mRNA retains at least 50% (e.g., about 50%, 60%, 70%, 80%, 90%, 92%, 94%, 96%, 98%, 99%, 99.5%, or more) of its original bioactivity (e.g., ability to express an encoded amino acid sequence) after storage for a period of at least about 1 hour, at least about 2 hours, at least about 3 hours, at least about 6 hours, at least about 12 hours, at least about 1 day, at least about 2 days, at least about 3 days, at least about 4 days, at least about 5 days, at least about 6 days, at least about 1 week, at least about 2 weeks, at least about 1 month, at least about 2 months, at least about 3 months, at least about 6 months or longer, e.g., at a temperature of about 4° C., about 25° C., about 37° C., and/or about 45° C.

In some embodiments, the mRNA retains at least 50% (e.g., about 50%, 60%, 70%, 80%, 90%, 92%, 94%, 96%, 98%, 99%, 99.5%, or more) of its original bioactivity (e.g., ability to express an encoded amino acid sequence) after storage for a period of 2 or more weeks, e.g., at a temperature of about 4° C., about 25° C., about 37° C., and/or about 45° C.

In some embodiments, the mRNA retains at least 50% (e.g., about 50%, 60%, 70%, 80%, 90%, 92%, 94%, 96%, 98%, 99%, 99.5%, or more) of its original bioactivity (e.g., ability to express an encoded amino acid sequence) after storage for a period of 4 or more weeks, e.g., at a temperature of about 4° C., about 25° C., about 37° C., and/or about 45° C.

In some embodiments, the mRNA retains at least 50% (e.g., about 50%, 60%, 70%, 80%, 90%, 92%, 94%, 96%, 98%, 99%, 99.5%, or more) of its original bioactivity (e.g., ability to express an encoded amino acid sequence) after storage for a period of 8 or more weeks, e.g., at a temperature of about 4° C., about 25° C., about 37° C., and/or about 45° C.

In some embodiments, the mRNA retains at least 50% (e.g., about 50%, 60%, 70%, 80%, 90%, 92%, 94%, 96%, 98%, 99%, 99.5%, or more) of its original bioactivity (e.g., ability to express an encoded amino acid sequence) after storage for a period of 16 or more weeks, e.g., at a temperature of about 4° C., about 25° C., about 37° C., and/or about 45° C.

In some embodiments, the mRNA retains at least 50% (e.g., about 50%, 60%, 70%, 80%, 90%, 92%, 94%, 96%, 98%, 99%, 99.5%, or more) of its original bioactivity (e.g., ability to express an encoded amino acid sequence) after storage for over about 1 year or longer, e.g., at a temperature of about 4° C., about 25° C., about 37° C., and/or about 45° C.

Additionally, the formulations, compositions, articles, microneedle and microneedle devices, and/or preparations described herein, including the implantable sustained-release tip formulations, are designed to not only sustain release of a therapeutic agent, such as an mRNA, over the duration, e.g., of tip retention in the dermis, but to also maintain stability of the mRNA during this period of time (e.g., at least about 1-2 weeks) in the dermis.

Accordingly, in some embodiments, the microneedles and microneedle devices described herein can improve the storage stability of mRNA therapeutics, including mRNA vaccines, by preventing and/or reducing degradation of an mRNA during prolonged storage. In particular embodiments, the microneedles and microneedle devices described herein can improve the storage stability of mRNA therapeutics, including mRNA vaccines, by preventing and/or reducing degradation of an mRNA during prolonged storage at a temperature of about 4° C., about 25° C., about 37° C., and/or about 45° C. In some embodiments, the microneedles and microneedle devices described herein can improve the storage stability of unstable mRNA therapeutics, including mRNA vaccines, by preventing and/or reducing degradation of an mRNA by at least about 50% (e.g., about 50%, 60%, 70%, 80%, 90%, 92%, 94%, 96%, 98%, 99%, 99.5%, or more) during prolonged storage at a temperature of about 4° C., about 25° C., about 37° C., and/or about 45° C., e.g., as compared to the mRNA therapeutic stored in the absence of a microneedle or microneedle device described herein. In some embodiments, the microneedles and microneedle devices described herein can improve the storage stability of unstable mRNA therapeutics, including mRNA vaccines, such that the mRNA retains at least about 50% (e.g., about 50%, 60%, 70%, 80%, 90%, 92%, 94%, 96%, 98%, 99%, 99.5%, or more) of its original bioactivity (e.g., ability to express an encoded amino acid sequence) after storage for a period of 2 or more weeks, e.g., at a temperature of about 4° C., about 25° C., about 37° C., and/or about 45° C. In some embodiments, the microneedles and microneedle devices described herein can improve the storage stability of mRNA coronavirus vaccines, e.g., by preventing and/or reducing degradation of an the mRNA coronavirus vaccine during prolonged storage at a temperature of about 4° C., about 25° C., about 37° C., and/or about 45° C. In some embodiments, the microneedles and microneedle devices described herein can improve the storage stability of mRNA influenza vaccines, e.g., by preventing and/or reducing degradation of an mRNA influenza vaccine during prolonged storage at a temperature of about 4° C., about 25° C., about 37° C., and/or about 45° C.

Accordingly, provided herein are various microneedles and microneedle devices comprising silk fibroin protein that are configured to stabilize an effective amount of a therapeutic agent, such as an mRNA (e.g., an mRNA vaccine) during storage and/or sustained release to a subject. In certain embodiments, provided herein are microneedle devices (e.g., a microneedle patches) comprising a plurality of microneedles, wherein the plurality of microneedles comprises: a microneedle comprising an mRNA, such as an mRNA vaccine, wherein the microneedle device is configured to deliver to a subject the mRNA in an amount sufficient to induce an immune response (e.g., a humoral and/or cellular immune response), and wherein the mRNA retains at least 50% (e.g., about 50%, 60%, 70%, 80%, 90%, 92%, 94%, 96%, 98%, 99%, 99.5%, or more) of its original bioactivity (e.g., ability to express an encoded amino acid sequence, such as a vaccine antigen) after storage for a period of at least about 1 hour, at least about 2 hours, at least about 3 hours, at least about 6 hours, at least about 12 hours, at least about 1 day, at least about 2 days, at least about 3 days, at least about 4 days, at least about 5 days, at least about 6 days, at least about 1 week, at least about 2 weeks, at least about 1 month, at least about 2 months, at least about 3 months, at least about 6 months or longer, e.g., at a temperature of about 4° C., about 25° C., about 37° C., and/or about 45° C.

Accordingly, provided herein are various methods for stabilizing a therapeutic agent, such as an mRNA (e.g., an mRNA vaccine) during prolonged storage and/or sustained release to a subject using the formulations, compositions, articles, microneedle and microneedle devices, and/or preparations described herein, including the implantable sustained-release tip formulations. In certain embodiments, the formulations, compositions, articles, microneedle and microneedle devices, and/or preparations described herein, including the implantable sustained-release tip formulations, can be used to stabilize an effective amount of a therapeutic agent, such as an mRNA (e.g., an mRNA vaccine) mRNA, such that the mRNA retains at least 50% (e.g., about 50%, 60%, 70%, 80%, 90%, 92%, 94%, 96%, 98%, 99%, 99.5%, or more) of its original bioactivity (e.g., ability to express an encoded amino acid sequence, such as a vaccine antigen) after storage for a period of at least about 1 hour, at least about 2 hours, at least about 3 hours, at least about 6 hours, at least about 12 hours, at least about 1 day, at least about 2 days, at least about 3 days, at least about 4 days, at least about 5 days, at least about 6 days, at least about 1 week, at least about 2 weeks, at least about 1 month, at least about 2 months, at least about 3 months, at least about 6 months or longer, e.g., at a temperature of about 4° C., about 25° C., about 37° C., and/or about 45° C.

Further, provided herein are various methods to prevent and/or reduce the degradation of a therapeutic agent, such as an mRNA (e.g., an mRNA vaccine) during prolonged storage and/or sustained release to a subject using the formulations, compositions, articles, microneedle and microneedle devices, and/or preparations described herein, including the implantable sustained-release tip formulations. In certain embodiments, the formulations, compositions, articles, microneedle and microneedle devices, and/or preparations described herein, including the implantable sustained-release tip formulations, can be used to prevent and/or reduce the degradation of a therapeutic agent, such as an mRNA (e.g., an mRNA vaccine) mRNA, by at least about 50% (e.g., about 50%, 60%, 70%, 80%, 90%, 92%, 94%, 96%, 98%, 99%, 99.5%, or more). Without wishing to be bound by theory, preventing and/or reducing the degradation of an mRNA enables using the formulations, compositions, articles, microneedle and microneedle devices, and/or preparations described herein, including the implantable sustained-release tip formulations, results in the mRNA retaining at least 50% (e.g., about 50%, 60%, 70%, 80%, 90%, 92%, 94%, 96%, 98%, 99%, 99.5%, or more) of its original bioactivity (e.g., ability to express an encoded amino acid sequence, such as a vaccine antigen) after storage for a period of at least about 1 hour, at least about 2 hours, at least about 3 hours, at least about 6 hours, at least about 12 hours, at least about 1 day, at least about 2 days, at least about 3 days, at least about 4 days, at least about 5 days, at least about 6 days, at least about 1 week, at least about 2 weeks, at least about 1 month, at least about 2 months, at least about 3 months, at least about 6 months or longer, e.g., at a temperature of about 4° C., about 25° C., about 37° C., and/or about 45° C. In particular embodiments, the mRNA which is stabilized encodes at least one vaccine, antigen, and/or immunogen (e.g., a coronavirus vaccine and/or an influenza vaccine) described herein.

Uses Provided herein are methods of treating, preventing, or alleviating viral infections or the symptoms caused by viral infection comprising administering to a subject in need thereof an effective amount of a therapeutic agent via a microneedle or the microneedle device as described herein.

The present disclosure features methods for delivering a vaccine, an antigen, and/or an immunogen (e.g., coronavirus vaccine and/or an influenza vaccine) across a biological barrier (e.g., the skin). Such methods can include providing a formulation, composition, article, device, preparation, and/or microneedle described herein.

For example, such methods can include providing at least one microneedle or at least one microneedle device described herein, wherein the microneedle or the microneedle device comprises an implantable tip (e.g., a silk fibroin-based implantable tip) comprising at least one vaccine, antigen, and/or immunogen (e.g., a coronavirus vaccine and/or an influenza vaccine); causing the microneedle or microneedle device to penetrate into the biological barrier (e.g., the skin); and allowing the vaccine, antigen, and/or immunogen to be released from the implantable tips over a period of at least about 4 days (e.g., about 4 to about 25 days, about 5 to about 25 days, about 10 to about 20 days, about 12 to about 18 days, about 14 to about 16 days, about 14 to about 15 days, e.g., about 4, 5, 6, 7, 8, 9, 10, 11, 12, 13, 14, 15, 16, 17, 18, 19, 20, 21, 22, 23, 24, or 25 or more days, e.g., between about 1-2 weeks, about 1-3 weeks, or about 1-4 weeks).

In some embodiments, the vaccine, antigen, and/or immunogen is released into the biological barrier through the degradation and/or dissolution of the implantable microneedle tips. In some embodiments, the microneedle or microneedle device is configured to administer the vaccine, antigen, and/or immunogen in an amount and/or a duration that results in broad-spectrum immunity in the subject, e.g., an immunity against one or more viral antigens not present in the implantable sustained-release tip, e.g., an immunity against a drifted strain not present in the implantable sustained-release tip.

The present disclosure also provides a method, microneedles, and/or microneedle devices for providing immunity to a virus, e.g., a coronavirus and/or an influenza virus, in a subject, said method comprising administering a vaccine, antigen, and/or immunogen (e.g., a coronavirus vaccine and/or an influenza vaccine) in an amount (e.g., a dosage) and/or over a time period sufficient to result in immunity to the virus, e.g., results in an immune response (e.g., a cellular immune response and/or a humoral immune response) to the virus, in the subject. In some embodiments, the vaccine is administered in a composition for the controlled- or sustained-release of the vaccine, e.g., a microneedle or microneedle device described herein, e.g., for the controlled- or sustained-release of one or more viral antigens as described herein). In some embodiments, the vaccine is administered by a device (e.g., a microneedle device) for the controlled- or sustained-release of the vaccine (e.g., for the controlled- or sustained-release of one or more viral antigens as described herein). The vaccine can be administered into a subject, e.g., into a tissue or cavity of the subject chosen from skin, mucosa, organ tissue, muscle tissue or buccal cavity.

In another aspect, the present disclosure provides a method, microneedle, and/or microneedle device for providing broad-spectrum immunity to a virus, e.g., a coronavirus and/or an influenza virus, in a subject, said method comprising administering a vaccine (e.g., a coronavirus vaccine and/or an influenza vaccine) in an amount (e.g., a dosage) and/or over a time period sufficient to result in broad-spectrum immunity to a virus, e.g., results in an immune response (e.g., a cellular immune response and/or a humoral immune response) to a drifted strain of the virus, in the subject. In some embodiments, the vaccine is administered in a composition for the controlled- or sustained-release of the vaccine, e.g., a microneedle or microneedle device described herein, e.g., for the controlled- or sustained-release of one or more viral antigens as described herein. In some embodiments, the vaccine is administered by a device for the controlled- or sustained-release of the vaccine (e.g., for the controlled- or sustained-release of one or more viral antigens as described herein). The vaccine can be administered into a subject, e.g., into a tissue or cavity of the subject chosen from skin, mucosa, organ tissue, muscle tissue or buccal cavity.

In some embodiments, the methods described herein comprise administering a vaccine, antigen, and/or immunogen (e.g., a coronavirus vaccine and/or an influenza vaccine) in an amount (e.g., a dosage) and/or over a time period sufficient to result in one or more of: (i) exposure in the subject to one or more antigens in the vaccine in an amount and/or period of time to result in broad spectrum immunity, e.g., to result in an immune response (e.g., a cellular immune response and/or a humoral immune response) to a drifted strain of the virus, in the subject; or (ii) a level of one or more antigens in the subject that is substantially steady, e.g., about 20%, 15%, 10%, 5%, or 1% to an amount, e.g., minimum amount, needed to result in an immune response (e.g., a cellular immune response and/or a humoral immune response) to the one or more antigens. In some embodiments, the composition or device for the controlled- or sustained-release of the vaccine is chosen from: a microneedle (e.g., a microneedle device, e.g., a microneedle patch, e.g., as described herein), an implantable device (e.g., a pump, e.g., a subcutaneous pump), an injectable formulation, a depot, a gel (e.g., a hydrogel), an implant, or a particle (e.g., a microparticle and/or a nanoparticle).

In some embodiments, the vaccine, antigen, and/or immunogen is administered, e.g., released by the composition or device for the controlled- or sustained-release of the vaccine, e.g., into the subject, in order to maintain a vaccine dosage (e.g., an antigen concentration) for a period of time sufficient to result in broad spectrum immunity, e.g., to result in an immune response (e.g., a cellular immune response and/or a humoral immune response) to a drifted strain of the virus, in the subject (e.g., wherein the period of time is about 1 to 25 days, e.g., about 1 to about 21 days, about 5 to about 25 days, about 10 to about 20 days, about 12 to about 18 days, about 14 to about 16 days, about 14 to about 15 days, about 5 to about 10 days, or about 5 to about 7 days, e.g., about 1, 2, 3, 4, 5, 6, 7, 8, 9, 10, 11, 12, 13, 14, 15, 16, 17, 18, 19, 20, 21, 22, 23, 24, or 25 days).

The composition or device for the controlled- or sustained-release of the vaccine, antigen, and/or immunogen, e.g., a microneedle or microneedle device described herein, can maintain vaccine, antigen, and/or immunogen release and/or level in the subject over a sustained period of time. In some embodiments the composition or device for the controlled- or sustained-release of the vaccine, antigen, and/or immunogen maintains a continuous or non-continuous vaccine, antigen, and/or immunogen release into the subject over a sustained period of time. The vaccine, antigen, and/or immunogen can administered, e.g., released by the composition or device for the controlled- or sustained-release, over a period of time comprising at least about one week, e.g., about 1-2 weeks, about 1-3 weeks, or about 1-4 weeks. In some embodiments, the vaccine is administered, e.g., released by the composition or device for the controlled- or sustained-release, over a period of time comprising at least about 4 days (e.g., about 4, 5, 6, 7, 8, 9, 10, 11, 12, 13, 14, 15, 16, 17, 18, 19, or 20 days, or more, e.g., between about 5 days and about 25 days, between about 10 days and about 20 days, between about 12 days and about 18 days, between about 14 days and about 16 days, between about 14 days and about 15 days, between about 4 days and about 2 weeks, or between about 4 days and about 1 week).

The vaccine, antigen, and/or immunogen can be administered in a dosage comprising between about 0.1 μg and about 1000 μg, e.g., between about 0.2 μg and about 750 μg, between about 0.2 μg and about 500 μg, between about 1 μg and about 250 μg, between about 1 μg and about 200 μg, between about 1 μg and about 150 μg, between about 1 μg and about 100 μg (e.g., about each of 0.1, 0.2, 0.3, 0.4, 0.5, 1, 2, 3, 4, 5, 6, 7, 8, 9, 10, 15, 20, 25, 50, 75, 100, 125, 150, 200, 250, 300, 350, 400, 450, 500, 750, or 1000 μg). In some embodiments, the vaccine, antigen, and/or immunogen (e.g., coronavirus vaccine and/or influenza vaccine) is administered in a dosage comprising between about 0.1 μg and about 500 μg, e.g., between about 0.2 μg and about 350 μg, between about 0.2 μg and about 300 μg, between about 1 μg and about 250 μg, between about 1 μg and about 200 μg, between about 1 μg and about 150 μg, between about 1 μg and about 100 μg (e.g., about each of 0.1, 0.2, 0.3, 0.4, 0.5, 1, 2, 3, 4, 5, 6, 7, 8, 9, 10, 15, 20, 25, 50, 75, 100, 125, 150, 200, 250, 300, 350, 400, 450, or 500 1000 μg). In some embodiments, the vaccine, antigen, and or immunogen (e.g., coronavirus vaccine and/or influenza vaccine) is administered in a dosage comprising between about 0.1 μg and about 65 μg per strain, e.g., 0.2 μg and about 50 μg per strain (e.g., about each of 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, or 65 μg per strain).

In some embodiments, at least about 1% of the dosage of the vaccine, antigen, and/or immunogen (e.g., at least about 0.5% to about 10%, at least about 5% to about 15% at least about 10% to about 20% of the dosage), e.g., released by the composition or device for the controlled- or sustained-release of the vaccine, e.g., into the subject, is maintained over a period of time comprising at least about 4 days (e.g., about 4, 5, 6, 7, 8, 9, 10, 11, 12, 13, or 14 days, or more, e.g., between about 5 days and about 25 days, between about 10 days and about 20 days, between about 12 days and about 16 days, between about 14 days and about 15 days, between about 4 days and about 2 weeks, between about 4 days and about 1 week).

In some embodiments, the vaccine, antigen, and/or immunogen is administered, e.g., released by the composition or device for the controlled- or sustained-release, in a plurality of fractional doses of a total dose (e.g., a standard dose) over a time period, e.g., such that an immune response and/or broad-spectrum immunity is achieved, wherein the amount of the vaccine, antigen, and/or immunogen administered in each of the fractional doses is no more than 1/X, wherein X is any number, e.g., wherein X is 0.1, 0.2, 0.3, 0.4, 0.5, 0.6, 0.7, 0.8, 0.9, 1, 2, 3, 4, 5, 6, 7, 8, 9, 10, 15, 20, 30, 40, 50, 60, 70, 80, 90, or 100 or more, of the total dose (e.g., a standard dose) of the vaccine, antigen, and/or immunogen.

In some embodiments, the vaccine, antigen, and/or immunogen is administered, e.g., released by the composition or device for the controlled- or sustained-release, e.g., into the skin of the subject, in a plurality of doses equivalent to a percentage of a total dose (e.g., a percentage of a standard dose) over a time period, e.g., such that broad-spectrum immunity is achieved, wherein the amount of the vaccine, antigen, and/or immunogen administered in each of the plurality of doses is about X %, wherein X is any number, e.g., wherein X is 0.1, 0.2, 0.3, 0.4, 0.5, 0.6, 0.7, 0.8, 0.9, 1, 2, 3, 4, 5, 6, 7, 8, 9, 10, 15, 20, 30, 40, 50, 60, 70, 80, 90, 100, 125, 150, 175, 200, 300, 400, or 500 or more, of the total dose (e.g., a standard dose) of the vaccine, antigen, and/or immunogen.

The vaccine, antigen, and/or immunogen can be administered according to any of the methods described herein such that broad-spectrum immunity is achieved, e.g., such that an immune response, e.g., a cellular immune and/or humoral immune response to a drifted strain of a virus is achieved.

Without wishing to be bound by theory, a subject exposed to and/or infected with a first virus (e.g., a coronavirus and/or an influenza virus), or exposed to an antigen corresponding to said virus (e.g., a coronavirus antigen and/or influenza antigen) can develop an immune response (e.g., a cellular immune and/or humoral immune response) resulting in the creation of an antibody against that first virus. For example, a neutralizing antibody, an immunoglobulin (e.g., immunoglobulin G (IgG), immunoglobulin M (IgM), immunoglobulin A (IgA)) specific to the virus may be created, e.g., an antibody described herein.

As antigenic changes (e.g., mutations) accumulate in the first virus over time, the subject's antibodies created against the first virus may no longer recognize the drifted virus (e.g., the antigenically different strain). Using the methods, dosage regimens, microneedles, and microneedle devices described herein, broad-spectrum immunity can be conferred to a subject exposed to, infected with, and/or at risk of infection with a virus (e.g., a coronavirus and/or an influenza virus). Further, using the methods, dosage regimens, microneedles, and microneedle devices described herein, improved immunogenicity and/or broad-spectrum immunity can be conferred to a subject, e.g., as compared to traditional burst release administration of vaccine. For example, improved immunogenicity and/or broad-spectrum immunity detectable in a subject can be greater (e.g., 1-fold, 2-fold, 3-fold, 4-fold, 5-fold, 6-fold, 7-fold, 8-fold, 9-fold, 10-fold, 11-fold, 12-fold, 13-fold, 14-fold, or 15-fold or more greater) as compared to traditional burst release administration of vaccine, e.g., the administration of a single-dose or a bolus administration of the vaccine.

In some embodiments, the microneedle (e.g., implantable sustained-release tip) or the vaccine comprises a coronavirus antigen associated with a first coronavirus strain, and administration of a dose of the coronavirus vaccine to the subject results in the development of broad-spectrum immunity to a second coronavirus strain (e.g., a drifted SARS-CoV-2 strain) not present in, associated with, or directly targeted by the microneedle or vaccine. In some embodiments, the microneedle, e.g., the implantable sustained-release tip, or the vaccine comprises a first coronavirus strain and administration of a dose of the first coronavirus strain (e.g., a first SARS-CoV, SARS-CoV-2, or MERS-CoV strain described herein) to the subject results in the development of broad-spectrum immunity to a second coronavirus strain (e.g., a drifted SARS-CoV, SARS-CoV-2, or MERS-CoV strain) not present in the microneedle or the vaccine.

In some embodiments, the implantable sustained-release tip or the vaccine comprises a first influenza strain and administration of a dose of the first influenza strain (e.g., a first influenza A, B, C, and/or D strain as described herein) to the subject results in the development of broad-spectrum immunity to a second influenza strain (e.g., a drifted influenza A, B, C, and/or D strain as described herein) not present in the implantable sustained-release tip or the vaccine.

In some embodiments, the subject (e.g., the human subject) is a pediatric subject, an adult subject, or an elderly subject. The subject may have been exposed to, infected with, and/or at risk of infection with a coronavirus and/or an influenza virus (e.g., a particular strain of a coronavirus and/or an influenza virus). Such a risk may be due to the health status or age of the subject and/or travel to a region where a particular strain of the virus is prevalent.

In some embodiments, the present disclosure provides methods of providing a controlled- or sustained-release of a vaccine, antigen, and/or immunogen in a subject. The controlled- or sustained-release of the vaccine, antigen, and/or immunogen can achieve an improved immunogenicity and/or broad-spectrum immunity, as compared to traditional burst release administration of vaccine, antigen, and/or immunogen. Without wishing to be bound by theory, an method of administering a vaccine, antigen, and/or immunogen described herein and/or a controlled- or sustained-release rate, e.g., by a composition and/or a microneedle described herein, that mimics the natural exposure pattern of a subject (e.g., a human subject) to a virus can provide enhanced immunity and/or broad-spectrum immunity to a subject, as compared to traditional single-dose vaccine administration modalities (e.g., a bolus dose administered subcutaneously or intranasally).

In some embodiments, a desired amount of at least one vaccine, antigen, and/or immunogen (e.g., a coronavirus vaccine and/or an influenza vaccine) can be released from the microneedle (e.g., implantable microneedle tip) described herein in a sustained manner over a pre-defined period of time. In some embodiments, at least about 5% of a vaccine, an antigen, and/or an immunogen (e.g., a coronavirus vaccine and/or an influenza vaccine), e.g., at least about 10%, at least about 15%, at least about 20%, at least about 30%, at least about 40%, at least about 50%, at least about 60%, at least about 70%, at least about 80%, at least about 90%, at least about 95%, or at least about 97%, about 98%, or about 99%, or 100% of the vaccine, antigen, and/or immunogen (e.g., a coronavirus vaccine and/or an influenza vaccine), can be released from the microneedle (e.g., implantable microneedle tips) over a pre-defined period of time. In such embodiments, the desired amount (e.g., a dose, such as a standard dose) of the vaccine, antigen, and/or immunogen (e.g., a coronavirus vaccine and/or an influenza vaccine) can be released from the microneedle over seconds, minutes, hours, months and/or years. In some embodiments, the desired amount (e.g., a dose, such as a standard dose) of the vaccine, antigen, and/or immunogen (e.g., a coronavirus vaccine and/or an influenza vaccine) can be released from the microneedle upon insertion into a biological barrier, e.g., within 5 seconds, within 10 seconds, within 30 seconds, within 1 minute, within 2 minutes, within 3 minutes, within 4 minutes, within 5 minutes or longer. In some embodiments, the desired amount (e.g., a dose, such as a standard dose) of the vaccine, antigen, and/or immunogen (e.g., a coronavirus vaccine and/or an influenza vaccine) can be released from the microneedle over a period of at least about 1 hour, at least about 2 hours, at least about 3 hours, at least about 6 hours, at least about 12 hours, at least about 1 day, at least about 2 days, at least about 3 days, at least about 4 days, at least about 5 days, at least about 6 days, at least about 1 week, at least about 2 weeks, at least about 1 month, at least about 2 months, at least about 3 months, at least about 6 months or longer. In some embodiments, the desired amount (e.g., a dose, such as a standard dose) of the vaccine, antigen, and/or immunogen (e.g., a coronavirus and/or an influenza vaccine) can be released from the microneedle over about 1 year or longer.

In some embodiments, the present disclosure provides methods for enhancing an immune response to a virus in a subject, e.g., by contacting the skin of the subject with a microneedle or microneedle device described herein. In some embodiments, the presence of a cell-mediated immunological response can be determined by any art-recognized methods, e.g., proliferation assays (CD4+ T cells), CTL (cytotoxic T lymphocyte) assays, or immunohistochemistry with tissue section of a subject to determine the presence of activated cells such as monocytes and macrophages after the administration of an immunogen.

One of skill in the art can readily determine the presence of humoral-mediated immunological response in a subject by any well-established methods. For example, the level of antibodies produced in a biological sample such as blood can be measured by western blot, ELISA, or other methods known for antibody detection. In some embodiments, the antibodies produced in a biological sample such as blood (e.g., from a finger prick) are measured using a lateral flow assay. In some embodiments, an antibody described herein (e.g., an antibody detected in a method described herein) is a coronavirus-specific antibody and/or an influenza-specific antibody. The antibody may be a neutralizing antibody. In some embodiments, the antibody is an anti-coronavirus neutralizing antibody. In some embodiments, the antibody is an anti-influenza neutralizing antibody. In some embodiments, the antibody is an immunoglobulin (e.g., immunoglobulin G (IgG), immunoglobulin M (IgM), or immunoglobulin A (IgA)). In some embodiments, the antibody is a broadly neutralizing antibody (bnAb).

The coronavirus-specific antibody may be a SARS-CoV-2-specific antibody, a SARS-CoV-specific antibody, or a MERS-CoV specific antibody. In some embodiments, the antibody is a SARS-CoV-2 specific antibody. In some embodiments, the antibody is a SARS-CoV-specific antibody. In some embodiments, the antibody is a MERS-CoV specific antibody. In some embodiments, the antibody is a coronavirus spike protein (S)-specific antibody. In some embodiments, the antibody is a spike-receptor binding domain (RBD)-specific antibody. In some embodiments, the antibody is a nucleocapsid (N)-specific antibody. In some embodiments, the antibody is a coronavirus-specific IgG, e.g., an anti-coronavirus IgG, such as a SARS-CoV-2 S-specific IgG, e.g., a SARS-CoV-2 S-specific IgG or a SARS-CoV-2 RBD-specific IgG. In some embodiments, the antibody is a SARS-CoV-2 S-specific IgG. In some embodiments, the antibody is a SARS-CoV-2 RBD-specific IgG.

In some embodiments, the antibody is a SARS-CoV-2 S-specific IgG antibody (also referred to herein as “a spike-specific IgG antibody”).

In certain embodiments, microneedle delivery of a coronavirus vaccine as described herein can result in a more robust and less variable spike-specific IgG antibody titers compared to traditional vaccine administration routes, such as via bolus intramuscular injections (IM) or bolus intradermal injection (ID).

In certain embodiments, single-dose microneedle delivery of a coronavirus vaccine as described herein may result in equivalent spike-specific IgG responses compared to IM prime/boost injections at lower doses (e.g., at half the total dose), thereby demonstrating a dose sparing effect.

In certain embodiments, microneedle delivery of an unadjuvanted coronavirus vaccine as described herein can improve spike-specific IgG responses compared to traditional vaccine administration routes, such as via bolus intramuscular injections (IM) or bolus intradermal injection (ID).

In certain embodiments, microneedle delivery of an unadjuvanted coronavirus vaccine as described herein can result in greater IgG titers than a 2-dose bolus IM regimen, at lower doses (e.g., at half the total dose), thereby demonstrating a dose sparing effect.

In certain embodiments, storage of the microneedle patches for prolonged periods of time, such as for at least about 8-weeks at room temperature, may result in the generation of equivalent spike-specific IgG responses as compared to freshly manufactured microneedles patches, thereby demonstrating advantageous shelf stability.

In some embodiments, the antibody is a hemagglutination inhibition (HAI) antibody. In some embodiments, the antibody is an anti-influenza IgG.

In some embodiments, an elevated coronavirus-specific antibody titer is detectable in the blood of the subject for the duration of a complete coronavirus season post-immunization. In some embodiments, an elevated coronavirus N-specific antibody titer (e.g., a SARS-CoV-2-N specific antibody) is detectable in the blood of the subject for the duration of a complete coronavirus season post-immunization. In some embodiments, an elevated coronavirus S-specific antibody titer (e.g., a SARS-CoV-2-S specific antibody) is detectable in the blood of the subject for the duration of a complete coronavirus season post-immunization.

In some embodiments, an elevated influenza-specific antibody titer is detectable in the blood of the subject for the duration of a complete flu season post-immunization. In some embodiments, an elevated hemagglutination inhibition (HAI) antibody titer is detectable in the blood of the subject for the duration of a complete flu season post immunization.

In some embodiments, the immune response and/or broad-spectrum immunity is a cellular and/or humoral immune response comprising: (i) an elevated coronavirus-specific antibody titer (e.g., a SARS-CoV-2-specific antibody, e.g., an antibody specific to a SARS-CoV-2 spike protein, or a subunit thereof) detectable in the blood of the subject, e.g., detectable at least 3, 4, 5, or 6 days, or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 51, and/or 52-weeks or more post immunization, (ii) an elevated anti-coronavirus IgG (e.g., anti-SARS-CoV-2 IgG) titer detectable in the blood of the subject, e.g., detectable at least 4, 5, or 6 days, or at least 1, 2, 3, or 4 weeks, or at least 1, 2, 3, 4, 5, 6, 7, 8, 9, 10, 11, and/or 12-months or more post immunization, and/or (iii) a level of antibody secreting plasma cells (ASC) against the coronavirus, e.g., the SARS-CoV-2 virus, detectable in the bone marrow of the subject, e.g., detectable at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 51, and/or 52-weeks or more post-immunization. In some embodiments, the elevated coronavirus-specific antibody titer is to a drifted coronavirus strain (e.g., a drifted SARS-CoV-2 strain). In some embodiments, the elevated anti-coronavirus IgG titer is to a drifted coronavirus strain (e.g., a drifted SARS-CoV-2 strain).

In some embodiments, an elevated coronavirus-specific antibody titer is detectable in the blood of the subject for at least 3, 4, 5, or 6 days or more post immunization. In some embodiments, an elevated coronavirus-specific antibody titer is detectable in the blood of the subject for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 51, and/or 52-weeks or more post immunization. In some embodiments, an elevated anti-coronavirus IgG titer is detectable in the blood of the subject for at least 4, 5, or 6 days or more post immunization. In some embodiments, an elevated anti-coronavirus IgG titer is detectable in the blood of the subject for at least 1, 2, 3, or 4 weeks or more post immunization. In some embodiments, an elevated anti-coronavirus IgG titer is detectable in the blood of the subject for at least 1, 2, 3, 4, 5, 6, 7, 8, 9, 10, 11, and/or 12-months or more post immunization. In some embodiments, the level of antibody secreting plasma cells (ASC) against the coronavirus, e.g., the SARS-CoV-2 virus, is detectable in the bone marrow of the subject for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 51, and/or 52-weeks or more post-immunization.

In some embodiments, the immune response and/or the broad-spectrum immunity is a cellular immune and/or humoral immune response comprising: (i) an elevated hemagglutination inhibition (HAI) antibody titer detectable in the blood of the subject, e.g., detectable at least 3, 4, 5, or 6 days, or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, and/or 30-weeks or more post immunization; (ii) an elevated anti-influenza IgG titer detectable in the blood of the subject, e.g., detectable at least 4, 5, or 6 days, or at least 1, 2, 3, or 4 weeks, or at least 1, 2, 3, 4, 5, 6, 7, 8, 9, 10, 11 and/or 12-months or more post immunization; and/or (iii) a level of antibody secreting plasma cells (ASC) against the virus, e.g., the influenza virus, detectable in the bone marrow of the subject, e.g., detectable at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, and/or 34-weeks or more post immunization. In some embodiments, the elevated HAI antibody titer is to a drifted influenza A, B, C, and/or D strain. In some embodiments, the elevated anti-influenza IgG titer is to a drifted influenza A, B, C, and/or D strain.

In some embodiments, an elevated hemagglutination inhibition (HAI) antibody titer is detectable in the blood of the subject for at least 3, 4, 5, or 6 days or more post immunization. In some embodiments, an hemagglutination inhibition (HAI) antibody titer is detectable in the blood of the subject for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 51, and/or 52-weeks or more post immunization. In some embodiments, an elevated anti-influenza IgG titer is detectable in the blood of the subject for at least 4, 5, or 6 days or more post immunization. In some embodiments, an elevated anti-influenza IgG titer is detectable in the blood of the subject for at least 1, 2, 3, or 4 weeks or more post immunization. In some embodiments, an elevated anti-influenza IgG titer is detectable in the blood of the subject for at least 1, 2, 3, 4, 5, 6, 7, 8, 9, 10, 11, and/or 12-months or more post immunization. In some embodiments, the level of antibody secreting plasma cells (ASC) against the influenza virus is detectable in the bone marrow of the subject for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 51, and/or 52-weeks or more post-immunization.

In some embodiments, the immune response is a cellular immune response comprising an increase in the level of IFNγ secreting cell in the blood of the subject, e.g., at least 1, 2, 3, 4, 5, 6, 7, 8, 9, 10, 11, and/or 12-weeks or more post immunization, e.g., by a microneedle described herein. In some embodiments, the immune response is a cellular immune response comprising an increase in the production of IFNγ per a preselected number of cells in the blood of the subject, e.g., at least 1, 2, 3, 4, 5, 6, 7, 8, 9, 10, 11, and/or 12-weeks post immunization, e.g., by a microneedle described herein. In some embodiments, IFNγ producing cells may be measured (e.g., detected) using ELISPOT, e.g., to determine an increase in IFNγ spot-forming units (SFU).

In some embodiments, the elevated coronavirus-specific antibody titer (e.g., a SARS-CoV-2-specific antibody, e.g., an antibody specific to a SARS-CoV-2 spike protein, or a subunit thereof), the elevated anti-coronavirus IgG titer, the level of antibody secreting plasma cells (ASC) against the coronavirus virus, the level of IFNγ secreting cells, and/or the production of IFNγ per a preselected number of cells, detectable in the subject is greater (e.g., 1-fold, 2-fold, 3-fold, 4-fold, 5-fold, 6-fold, 7-fold, 8-fold, 9-fold, 10-fold, 11-fold, 12-fold, 13-fold, 14-fold, or 15-fold or more greater) as compared to the administration of a single-dose or a bolus administration of the vaccine.

In some embodiments, the elevated HAI antibody titer, the elevated anti-influenza IgG titer, the level of antibody secreting plasma cells (ASC) against the virus, the level of IFNγ secreting cells, and/or the production of IFNγ per a preselected number of cells, detectable in the subject is greater (e.g., 1-fold, 2-fold, 3-fold, 4-fold, 5-fold, 6-fold, 7-fold, 8-fold, 9-fold, 10-fold, 11-fold, 12-fold, 13-fold, 14-fold, or 15-fold or more greater) as compared to the administration of a single-dose or a bolus administration of the vaccine.

In some embodiments, broad-spectrum immunity can be characterized by measuring the percent seroconversion in a subject. In some embodiments, broad-spectrum immunity comprises a percent seroconversion, e.g., based on the elevated coronavirus-specific antibody titer detectable in the blood of the subject, e.g., at 6-month post immunization greater than about 20% (e.g., 20%, 25%, 30%, 35%, 40%, 45%, 50%, 55%, 60%, 65%, 70%, 75%, 80%, 85%, 90%, or 95% or more, e.g., 100%). In some embodiments, broad-spectrum immunity comprises a percent seroconversion, e.g., based on the elevated HAI antibody titer detectable in the blood of the subject, e.g., at 6-month post immunization greater than about 20% (e.g., 20%, 25%, 30%, 35%, 40%, 45%, 50%, 55%, 60%, 65%, 70%, 75%, 80%, 85%, 90%, or 95% or more, e.g., 100%). In some embodiments, seroconversion is a greater than 1-fold increase from baseline coronavirus-specific antibody titer, and/or influenza-specific antibody titer (e.g., HAI antibody titer) e.g., a 2-fold, 3-fold, 4-fold, 5-fold, or 6-fold or greater increase from baseline coronavirus-specific antibody titer and/or influenza specific antibody titer. In some embodiments, seroconversion is a four-fold increase from baseline coronavirus-specific antibody titer, e.g., an increase of 10 to 40. In some embodiments, seroconversion is a four-fold increase from baseline HAI antibody titer, e.g., an increase of 10 to 40.

Such a level of seroconversion associated with broad-spectrum immunity conferred by using the methods, dosage regimens, microneedles, and microneedle devices described herein can be greater (e.g., 1-fold, 2-fold, 3-fold, 4-fold, 5-fold, 6-fold, 7-fold, 8-fold, 9-fold, 10-fold, 11-fold, 12-fold, 13-fold, 14-fold, or 15-fold or more greater) as compared to a level of seroconversion obtained by traditional burst release administration of vaccine (e.g. the coronavirus vaccine and/or the influenza vaccine), e.g., the administration of a single-dose or a bolus administration of the vaccine (e.g., administered subcutaneously or intranasally).

The methods described herein can involve administering a coronavirus vaccine and/or an influenza vaccine prophylactically, e.g., using a microneedle or microneedle device described herein. In some embodiments, a microneedle or microneedle device (e.g., microneedle patch) described herein provides single-dose protection against infection against a virus (e.g., coronavirus and/or influenza virus), in the subject. For example, a coronavirus vaccine and/or influenza vaccine described herein may be administered as a patch (e.g., a microneedle patch described herein), e.g., a single patch. The patch can be administered by a health care professional, such as a doctor, nurse, or any suitable health care professional. Alternatively, the patch may be self-administered. For example, the patch may be provided to the subject, e.g., in an appropriate storage device, and the subject can self-administer the patch, e.g., from home or without needing to visit a clinic. The subject may wear the patch for a period of time of less than 1 hour, e.g., about 1 minute to about 45 minutes, about 2 minutes to about 30 minutes, about 5 minutes to about 15 minutes, e.g., about 5 minutes. In some embodiments, the subject wears the patch for about 5 minutes.

In some embodiments, the patch is administered using an applicator, e.g., an applicator device suitable for administering a microneedle device (e.g., microneedle patch) described herein.

In some embodiments, the microneedle device (e.g., microneedle patch) comprises, and/or is configured to release, a non-vaccine molecule described herein, e.g., a dye molecule, that allows confirmation of dose-delivery in a subject. For example, following administration of a microneedle device (e.g., microneedle patch) comprising a vaccine, antigen, and/or immunogen (e.g., a coronavirus vaccine and/or influenza vaccine), in addition to a non-vaccine molecule (e.g., dye), in a subject, the delivery of the vaccine, antigen, and/or immunogen may be confirmed by detection of the non-vaccine molecule, e.g., in the skin of the subject, such as by illumination of the non-vaccine molecule under UV irradiation, or any other suitable means of detection.

In some embodiments, the microneedle patch comprising the vaccine antigen, and/or immunogen is applied to the subject (e.g., worn by the subject) seasonally. For example, the patch may be applied seasonally, such that the coronavirus vaccine and/or the influenza vaccine is administered once per coronavirus season and/or influenza season. In some embodiments, the coronavirus vaccine and/or the influenza vaccine is administered on a regular booster schedule, e.g., yearly.

In another aspect, the present disclosure features methods, microneedles, and microneedle devices for protection against diseases caused by a coronavirus and/or an influenza virus, e.g., for the duration of a coronavirus or influenza season. In some embodiments, the microneedle or microneedle device protects (e.g., prevents) a subject from developing coronavirus disease 2019 (COVID-19). In some embodiments, the microneedle or microneedle device protects (e.g., prevents) a subject from developing Severe Acute Respiratory Syndrome (SARS). In some embodiments, the microneedle or microneedle device protects (e.g., prevents) a subject from developing Middle East Respiratory Syndrome (MERS). In some embodiments, the microneedle or microneedle device protects (e.g., prevents) a subject from developing influenza. In some embodiments, the microneedle or microneedle device protects (e.g., prevents) a subject from developing both coronavirus disease 2019 (COVID-19) and influenza.

### Exemplary Kits

In certain embodiments, the present disclosure features a package or kit comprising a microneedle, or a microneedle device (e.g., microneedle patch), as described herein (e.g., a microneedle including a vaccine, antigen, and/or an immunogen as described herein, such as a coronavirus vaccine and/or an influenza virus vaccine). In some embodiments, the present disclosure relates to a package or kit comprising a vaccine described herein (e.g., a vaccine, antigen, and/or an immunogen as described herein, such as a coronavirus vaccine and/or an influenza vaccine). In some embodiments, the kit can further comprise an additional therapeutic for combination therapy with the microneedle. In some embodiments, the kit can further comprise a disinfectant (e.g., an alcohol swab). In some embodiments, the kit can further comprise instructions, e.g., instructions useful for the application or administration of a microneedle device described herein. In some embodiments, such packages, and kits described herein can be used for vaccination purposes, e.g., to achieve broad-spectrum immunity in a subject as described herein.

### EXAMPLES

In order that the invention described herein may be more fully understood, the following examples are set forth. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Coronavirus antigens to be used in the following examples may include, but are not limited to, SARS-CoV-2 antigens, SARS-CoV antigens, or MERS-CoV antigens. For example, coronavirus spike protein, an inactivated virus; mRNA encoding a coronavirus protein, an adenovirus vector that expresses a coronavirus protein, a DNA plasmid encoding a coronavirus protein, a dendritic cell modified to express a coronavirus gene, or an artificial antigen-presenting cell (aAPC) modified to express a coronavirus gene. An exemplary list of coronavirus antigens to be investigated include: a pre-fusion SARS-CoV-2 spike protein, a UV inactivated SARS-CoV-2; mRNA-1273; Ad5-nCoV; Ad26 SARS-CoV-2, INO-4800; and LV-SMENP-DC.

Influenza antigens to be used in the following examples may include, but are not limited to, monovalent, bivalent, trivalent, and quadrivalent influenza vaccines, including influenza A, influenza B, influenza C, and/or influenza D antigens. The influenza vaccines used in these examples may be inactivated influenza virus vaccines. For example, Fluzone High-Dose may be used, or any other influenza antigen described herein.

### Example 1. Sustained Release Microneedle Formulation and Fabrication

A coronavirus vaccine and an influenza vaccine will be prepared for microneedle device fabrication. If necessary, either the influenza vaccine and/or the coronavirus vaccine will be first processed to remove excess detergent and to concentrate the antigens. In order to do so, multiple doses (e.g., 10 doses) of either the coronavirus vaccine or influenza vaccine will be run serially through a detergent removal column (e.g., Pierce™ Detergent Removal Spin Column) to remove detergent (such as Triton X-100, which is a common byproduct of manufacturing used to inactivate (e.g., split) virus). To confirm absence of detergent, an aliquot of material will be collected and analyzed by size exclusion chromatography (HPLC-SEC). The remaining material will be concentrated in 10 kDa spin filters (e.g., Amicon Ultra 0.5 mL, Fischer Sci 501096) through up to 3 10-minute spins at 15000 rpm. An aliquot of material will be run on HPLC-SEC to determine concentration of antigens against initial vaccine. Comparison of area-under-the-curve (AUC) for pre-concentration and post-concentration material will be used to determine the concentration of the processed antigen stock.

For each antigen, about 100 uL of stock (or any appropriate volume) will be mixed with silk fibroin (e.g., about 85.6 uL of silk fibroin (60 MB)) and Milli-Q water (e.g., 64.4 uL) to generate silk fibroin antigen solution (e.g., about 5% (w/v)), which will be printed into microneedle molds. If desired, the vaccines will be co-formulated prior to needle-tip printing, e.g., to provide microneedles co-formulated with one or more coronavirus and/or influenza vaccine. Otherwise, antigen solutions may be kept separate, and individually printed into microneedle molds, for example into separate wells of the mold.

Tip Filling: 20 nL of formulation will be printed using vision-guided dispensing (Biodot AD3420) into a polydimethylsiloxane (PDMS) microneedle mold.

Tip Fill Inspection: Printing will be visually assessed under stereomicroscope for defects, including misaligned prints, incompletely filled needle cavities, and foreign debris.

Tip Dry: Filled microneedle molds will be dried under controlled conditions, e.g., under 20% relative humidity, overnight (approximately 14-20 hours).

Tip Anneal: Dried tips may be water annealed at 37° C. for four hours, through placement of molds in a vacuum desiccator filled with Milli-Q water, applying vacuum for 5 minutes, then closing vacuum valve and moving desiccator to 37° C. incubator.

Tip Dry: after annealing tips will be again dried under controlled conditions, e.g., under 20% relative humidity, overnight (approximately 14-20 hours).

Base Filling: The desired base layer solution will then be filled into the microneedle molds. For example, 40% (w/v) hydrolyzed gelatin (Gelita) and 10% (w/v) sucrose (Sigma-Aldrich) will be pipetted onto microneedle molds and filled via centrifugation at 3900 rpm for 2 minutes.

Base Fill Inspection: Base filling will be assessed visually by stereomicroscope for the appearance of needle cavities that were not entirely filled. Re-filling and re-centrifugation will be performed if lack of fill is observed.

Base Drying: Base solution will be dried under controlled conditions, e.g., 20% relative humidity overnight (14-20 hours).

Backing Apply: A suitable backing layer will then be applied to the bases (e.g., using paper or adhesive tape). For example, Whatman 903 cards will be punched into 12 mm discs and applied to the pre-wetted (10 uL Milli-Q water) dried gelatin base.

Backing Dry: The devices will then be dried under controlled conditions, e.g., 20% relative humidity conditions for 2 hours, before demolding.

Demolding: Devices will be manually removed from microneedle molds by carefully bending the mold away from the device while holding device stationary.

Demold Inspect: Devices will be inspected for complete demolding under stereomicroscope; incompletely demolded devices will be discarded.

### Example 2. In Vivo Evaluation of a Microneedle Device for the Administration of Coronavirus Antigen and One or More Influenza Antigens

An in vivo model, e.g. using mice (e.g., BALB/c mice) will be used to assess the sustained release of a coronavirus antigen (e.g., a coronavirus vaccine) and one or more influenza antigens, in their ability to elicit an immune response (e.g., a cellular and/or humoral immune response), and/or provide immunity (e.g., broad-spectrum immunity) to the coronavirus.

The coronavirus antigen and influenza antigen(s) will be administered intradermally or subcutaneously either as:
1. a single bolus,
2. a series of fractional injections where the total dose given over the time frame is equivalent to the single bolus. These injections may be given daily or every other day.
3. microneedle patch (e.g., a silk fibroin microneedle patch) formulated with the coronavirus antigen and the influenza antigen.

The duration of sustained release (daily injections or microneedles) will be explored and optimized (˜2-28 days). Additionally, it is possible that multiple cycles of sustained released are optimal for providing immunity. To test this, the antigen will be administered via either single bolus or sustained release (daily injections or microneedles) over ˜2-28 days. For a period of time (˜5-14 days), animals will be given no treatment and then given the antigen in a second round identical to the first.

The antibody titer, e.g., anti-flu IgG and anti-coronavirus IgG responses will be measured by ELISA, or a suitable alternative. Titers will also be measured several weeks post-immunization, e.g., at days 28 and 56 post immunization. T cell responses following vaccination will also be measured, e.g., at week 12 by ELISPOT, or any suitable alternative. The results between different modes of administration (i.e., either of 1-3 listed above) will be compared to determine the degree of immunogenicity, and to determine that sustained delivery of a vaccine against coronavirus and/or influenza results in stronger humoral and cellular responses than equivalent dose delivered by conventional intramuscular injections.

### Example 3. In Vivo Evaluation of the Efficacy of Silk-Based Microneedle Administration of Coronavirus Vaccines

An in vivo model (e.g., using a mouse, e.g., BALB/c mouse) will be used to assess the efficacy of sustained release of a coronavirus antigen (e.g., a coronavirus vaccine) from a silk-fibroin-based microneedle patch, and the ability to elicit an immune response, and/or provide immunity (e.g., broad-spectrum immunity) to the coronavirus.

The coronavirus antigen will be administered intradermally or subcutaneously either as:
1. a single bolus,
2. a series of fractional injections where the total dose given over the time frame is equivalent to the single bolus. These injections may be given daily or every other day.
3. a silk microneedle patch formulated with the coronavirus antigen.

As in Example 2, anti-coronavirus IgG responses will be measured by ELISA, or a suitable alternative. Titers will also be measured several weeks post-immunization, e.g., at days 28 and 56 post immunization. T cell responses following vaccination will be measured, e.g., at week 12 by ELISPOT, or any suitable alternative. The results between different modes of administration (either of 1-3 outlined above) will be compared to determine the degree of immunogenicity, and to determine that sustained delivery of a coronavirus antigen from a silk fibroin-based microneedle patch results in stronger humoral and cellular responses than equivalent dose delivered by conventional intramuscular injections.

### Example 4. In Vivo Evaluation of Tip Separation and Antigen Kinetics

An in vivo model (e.g., using a mouse, e.g., BALB/c mouse) will be used to assess the tip separation and antigen kinetics of a microneedle patch, e.g., a silk-fibroin-based microneedle patch loaded with one or more coronavirus antigen and/or one or more influenza antigen. The results will be contrasted to a subject, e.g., BALB/c mouse, administered a bolus dose (e.g., intradermal bolus dose) of the fluorescently-labeled antigen.

The coronavirus antigen and/or influenza antigen will be fluorescently labeled with Alexafluor 647 (AF647, Invitrogen), or a suitable alternative. The labeled antigen will then be loaded into the microneedle patch, e.g., following the protocol outlined in Example 1. The microneedle patch comprising the fluorescently labeled antigens will be imaged via fluorescence microscope (EVOS FL, ThermoFisher), or a suitable alternative, before and after administration. The imaging will permit examination of the localization of antigen within the microneedle tips, and to confirm tips are properly released from the dissolving polymer in the skin after application. Whole animal fluorescent imaging will be performed with IVIS (PerkinElmer), or a suitable alternative, to track the retention of the coronavirus antigen and/or influenza antigen in the skin over a sufficient period of time for the antigen to be released from the implanted tips, such as over a period of two-weeks.

The effect of sustained release from the intradermal microneedle tips on antigen trafficking to draining lymph nodes will also be examined. Vaccination site-draining lymph nodes, such as inguinal lymph nodes, will be excised from the subject (e.g., mice) approximately three days after immunization with the microneedle patch containing the fluorescently labeled antigens. The lymph nodes will be tissue cleared using the iDISCO protocol (Renier et al. Cell (2014) 159:896-910), or a suitable alternative, and volume-imaged, e.g., using confocal microscopy. Antigen localization within the draining lymph nodes will be examined and compared to the localization effects in draining lymph nodes excised from subjects that received bolus injection (e.g., bolus intradermal) of the fluorescently labeled coronavirus antigen and/or influenza antigen).

### Example 5. Sustained Intradermal Delivery of SARS-CoV-2 Recombinant Protein Vaccine Generates Enhanced Humoral Antibody Responses

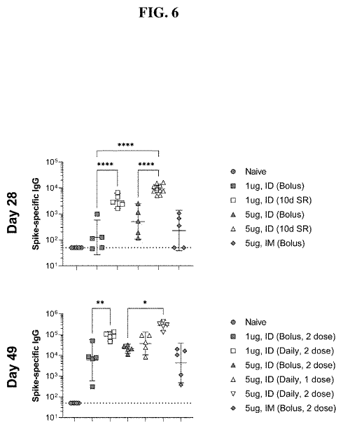

Balb/c mice (n=5/group) were immunized with either 1 μg or 5 μg unadjuvanted prefusion stabilized SARS-CoV-2 spike protein antigen, also referred to as “SARS-CoV-2 S-2P” or “S-2P” (Medigen Vaccine Biologics Corporation), twice at 4-week intervals via bolus intramuscular injection (IM), bolus intradermal injection (ID), or equivalent daily fractional doses ID across 10 days to simulate sustained release kinetics. A cohort of the 5 μg fractional dose group was only immunized on day 0 to evaluate a single vs two-dose sustained release immunization. Blood was collected at monthly time points, and spike-specific IgG titers were measured to evaluate humoral immune response (FIG. 6). These results demonstrate that sustained ID delivery of unadjuvanted prefusion stabilized SARS-CoV-2 spike protein antigen resulted in significantly higher spike-specific IgG titers compared to equivalent bolus IM or bolus ID injections. In addition, a single sustained release dose outperformed two bolus IM injections of unadjuvanted prefusion stabilized SARS-CoV-2 spike protein antigen, demonstrating a dose-sparing effect.

### Example 6. Immunization Via Sustained-Release Silk Microneedles Improves Humoral Immune Responses

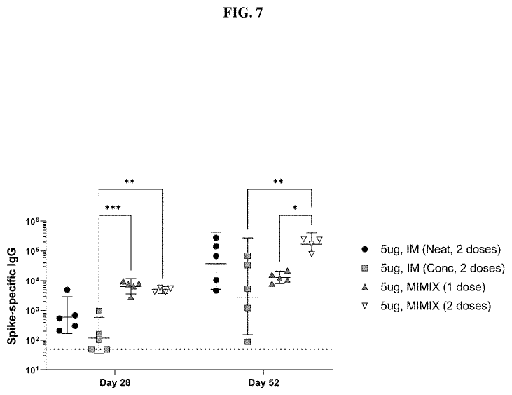

To generate COVID vaccine microneedle patches (MIMIX), recombinant pre-fusion stabilized SARS-CoV-2 spike protein (S-2P) was prepared for microneedle device fabrication by concentrating material in 10 kDa spin filter for 60 minutes at 14000×g for 60 minutes. An aliquot of material was run on ELISA to determine concentration of antigen against the initial vaccine. Liquid formulation containing concentrated S-2P protein was mixed with silk and other excipients, dispensed into a needle shaped cavity mold using a piezoelectrically actuated jetting valve, and dried under controlled humidity and temperature to tune silk crystallinity and solubility. Once dry, a soluble base formulation was dispensed into the mold and dried before a tape backing was applied to the device to demold. To evaluate immune responses of these devices, Balb/c mice (n=5/group) were immunized with 5 μg SARS-CoV-2 S-2P twice at 4-week intervals via bolus intramuscular injection (IM; neat or concentrated vaccine) or via microneedle devices (MIMIX; concentrated vaccine). MIMIX devices were applied manually via thumb press on a hairless section on the mouse flank, and removed after 5 minutes. Blood was collected at monthly time points, and spike-specific IgG titers were measured to evaluate humoral immune response (FIG. 7). These results demonstrate that MIMIX delivery of unadjuvanted S-2P improves spike-specific IgG responses compared with IM injection. Further, a single MIMIX administration resulted in greater IgG titers than a 2-dose bolus IM regimen, at half the total dose, demonstrating a dose sparing effect.

### Example 7. Evaluation of Alternate Formulations and Doses Delivered Via Silk-Microneedles

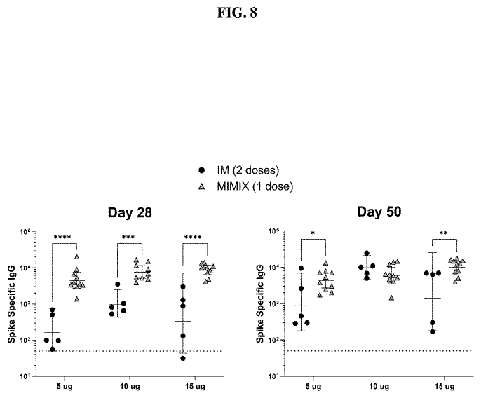

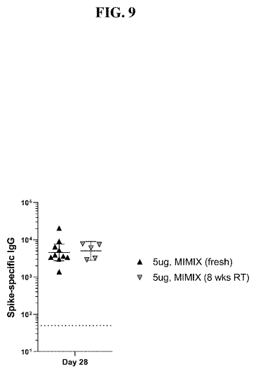

Balb/c mice were immunized with 5 μg, 10 μg, or 15 μg SARS-CoV-2 S-2P twice at 4-week intervals via bolus intramuscular injection (IM) or once via microneedle devices (MIMIX). In addition, a cohort of patches were stored at room temperature at 10% RH for 8 weeks to evaluate stability of MIMIX patches in vivo. MIMIX devices were fabricated and administered to mice as previously described. Blood was collected at monthly time points and spike-specific IgG antibody titers were measured to evaluate humoral immune response (FIGS. 8-9). These results demonstrate that MIMIX delivery of 5 μg S-2P resulted in improved spike-specific IgG titers compared to IM injection. When evaluating dose, MIMIX delivery resulted in more robust and less variable IgG titers compared to IM injections at the 5 μg, 10 μg, and 15 μg dose level (FIG. 8). Additionally, single-dose MIMIX immunization generated equivalent spike-specific IgG responses to IM prime/boost at half the total dose (FIG. 8). Finally, MIMIX COVID patches stored at 8-weeks generated equivalent spike-specific IgG responses to freshly manufactured patches demonstrating shelf stability (FIG. 9).

### EQUIVALENTS AND SCOPE

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, Figures, or Examples but rather is as set forth in the appended claims. Those of ordinary skill in the art will appreciate that various changes and modifications to this description may be made without departing from the spirit or scope of the present invention, as defined in the following claims.

1. A microneedle device comprising a plurality of microneedles, wherein the plurality of microneedles comprises:
a first microneedle comprising a coronavirus antigen and/or a coronavirus vaccine; and
optionally, a second microneedle comprising an influenza antigen and/or an influenza vaccine;
wherein the microneedle device is configured to deliver to a subject the coronavirus antigen and/or the coronavirus vaccine and, optionally, the influenza antigen and/or the influenza vaccine, in an amount sufficient to induce an immune response.

2.-4. (canceled)

5. A microneedle device comprising a plurality of silk fibroin-based microneedles, wherein:
the plurality of microneedles comprises a coronavirus antigen and/or a coronavirus vaccine; and
wherein the microneedle device is configured to deliver to a subject the coronavirus antigen and/or the coronavirus vaccine, in an amount sufficient to induce an immune response.

6. The microneedle device of claim 5, wherein the microneedle comprises:
(i) a dissolvable base;
(ii) an implantable silk fibroin tip applied to the base; and
(iii) optionally a backing applied to the dissolvable base.

7. The microneedle device of claim 5, wherein the microneedle device further comprises a second microneedle or a plurality of microneedles comprising an influenza antigen and/or an influenza vaccine.

8. The microneedle device of claim 7 wherein:
(i) the microneedle tip comprises the coronavirus antigen and/or the coronavirus vaccine;
(ii) the microneedle tip comprises the influenza antigen and/or the influenza vaccine;
(iii) the dissolvable base comprises the coronavirus antigen and/or the coronavirus vaccine;
(iv) the dissolvable base comprises the influenza antigen and/or the influenza vaccine;
(v) the first microneedle and/or the second microneedle comprises one antigen per microneedle or one vaccine per microneedle;
(vi) the first microneedle comprises a combination of antigens derived from the same coronavirus, or from different coronaviruses; and/or
(vii) the second microneedle comprises a combination of antigens derived from the same influenza virus, or from different influenza viruses.

9.-13. (canceled)

14. The microneedle device of claim 1, wherein the device or plurality further comprises a plurality of additional microneedles, wherein one or more microneedles in the plurality of additional microneedles comprises an influenza vaccine.

15.-21. (canceled)

22. The microneedle device of claim 1, wherein a portion of or each of the microneedles in the plurality comprise the coronavirus antigen and the influenza antigen.

23. The microneedle device of claim 22, wherein all of the microneedles in the plurality are the same.

24. The microneedle device of claim 1, wherein each of the coronavirus antigen and the influenza antigen is independently formulated into the same microneedle or into separate microneedles.

25. The microneedle device of claim 24, wherein each microneedle comprising at least one different antigen.

26.-29. (canceled)

30. The microneedle device of claim 1, wherein at least 10% of the microneedles independently comprise the coronavirus antigen and/or the influenza antigen.

31. The microneedle device of claim 1, wherein the coronavirus vaccine comprises a SARS-CoV-2 antigen, a MERS-CoV antigen, a SARS-CoV antigen, or a combination thereof.

32. (canceled)

33. (canceled)

34. The microneedle device of claim 1, wherein the coronavirus vaccine is selected from the group consisting of a coronavirus spike protein or a portion thereof, an inactivated coronavirus or a portion thereof, an mRNA encoding a coronavirus protein or a portion thereof, an adenovirus vector capable of expressing a coronavirus protein or a portion thereof, a DNA plasmid encoding a coronavirus protein or a portion thereof, a dendritic cell modified to express a coronavirus minigene, an artificial antigen-presenting cell (aAPC) modified to express a coronavirus minigene, and a combination thereof.

35. (canceled)

36. The microneedle device of claim 1, wherein the coronavirus vaccine comprises:
(i) a whole spike protein, a stabilized spike protein, a locked spike protein, a spike protein subunit, and/or a receptor-binding domain (RBD) from a spike protein;
(ii) a nucleic acid molecule, a vector, a plasmid, an adenovirus vector, an RNA molecule, an mRNA, an saRNA, a modRNA, a uRNA, and/or a DNA molecule that encodes a coronavirus spike protein or portion thereof;
(iii) an inactivated SARS-CoV-2;
(iv) a UV inactivated SARS-CoV-2;
(v) a recombinant SARS-CoV-2 spike protein or a subunit thereof;
(vi) a pre-fusion SARS-CoV-2 spike protein;
(vii) a coronavirus-derived protein;
(viii) a lipid nanoparticle (LNP) formulation;
(ix) an mRNA encapsulated by an LNP; and/or
(x) mRNA-1273, BNT162, INO-4800, Ad26 SARS-CoV-2, TNX-1800, PiCoVacc, or a combination thereof.

37.-41. (canceled)

42. The microneedle device of claim 1, wherein the influenza vaccine comprises a univalent or multivalent influenza vaccine.

43. (canceled)

44. The microneedle device of claim 1, wherein:
(i) the plurality of microneedles comprises a third microneedle comprising an influenza antigen that is different from the influenza antigen in the second microneedle;
(ii) the plurality of microneedles comprises a fourth microneedle comprising an influenza antigen that is different from the influenza antigen in the second and third microneedles;
(iii) the plurality of microneedles comprises a fifth microneedle comprising an influenza antigen that is different from the influenza antigen present in the second, third, and fourth microneedles;
(iv) the combination of influenza antigens in the second and third microneedle comprises a bivalent influenza vaccine;
(v) the combination of influenza antigens in the second, third, and fourth microneedle comprises a trivalent influenza vaccine; and/or
(vi) the combination of influenza antigens in the second, third, fourth, and fifth microneedles comprises a quadrivalent influenza vaccine.

45.-47. (canceled)

48. The microneedle device of claim 1, wherein:
(i) the influenza vaccine comprises an influenza A vaccine, an influenza B vaccine, an influenza C vaccine, and/or an influenza D vaccine;
(ii) the influenza vaccine comprises an influenza A vaccine comprising a H1N1 vaccine and/or a H3N2 vaccine;
(iii) the influenza vaccine comprises an influenza B vaccine comprising a B/Yamagata lineage vaccine and/or a B/Victoria lineage vaccine;
(iv) the plurality of microneedles comprises a 4:1 ratio of influenza vaccines to coronavirus vaccine; and/or
(v) the combination of the coronavirus vaccine and influenza vaccines comprises a pentavalent vaccine.

49.-51. (canceled)

52. The microneedle device of claim 1, wherein:
(i) the microneedle device is configured to result in a single dose immunity to a coronavirus and/or influenza virus;
(ii) the microneedle device is configured to deliver to the subject the coronavirus vaccine and/or the influenza vaccine in an amount sufficient to induce an immune response;
(iii) the microneedle device is configured for sustained release of the coronavirus vaccine and/or the influenza vaccine;
(iv) the microneedle device is configured to sustain a substantially continuous low dose administration of the coronavirus vaccine and/or the influenza vaccine;
(v) the microneedle device is configured to release between about 1 μg to about 500 μg of the coronavirus vaccine and/or the influenza vaccine over a period of time comprising at least about 4 days; and/or
(vi) the microneedle device is configured to release the coronavirus vaccine and/or the influenza vaccine into the skin of a subject over a period of time comprising at least about 4 days.

53. (canceled)

54. The microneedle device of claim 6, wherein:
(i) the silk fibroin comprises a regenerated silk fibroin and/or a recombinant silk fibroin;
(ii) the microneedles comprise at least about 1%, 2%, 3%, 4%, 5%, 6%, 7%, 8%, 9%, 10%, 11%, 12%, 13%, 14%, 15%, 16%, 17%, 18%, 19%,20%, 21%,22%, 23%, 24%, or 25% or more of a dose of the coronavirus vaccine; and/or
(iii) the silk fibroin tip comprises silk fibroin at a concentration of about 1% w/v to about 10% w/v.

55.-59. (canceled)

60. The microneedle device of claim 1, wherein the dissolvable base comprises two or more of:
(i) a polysaccharide;
(ii) a disaccharide;
(iii) a polymer;
(iv) a protein;
(v) a plasticizer; and
(vi) a surfactant.

61. The microneedle device of claim 6, wherein the base comprises one or more of gelatin, dextran, glycerol, polyethylene glycol (PEG), sucrose, trehalose, maltose, carboxymethylcellulose (CMC), polyvinylpyrrolidone (PVP), polyvinyl alcohol (PVA), hyaluronate, methyl cellulose, a octyl phenol ethoxylate, Triton-X, polysorbate, a poloxamer, P188, and/or a polyethoxylated alcohol.

62. (canceled)

63. The microneedle device of claim 6, wherein the implantable silk fibroin tip comprises:
(i) a disaccharide;
(ii) a polymer;
(iii) an amino acid;
(iv) a plasticizer;
(v) a buffer;
(vi) a surfactant; and/or
(vii) an adjuvant.

64. The microneedle device of claim 1, wherein the microneedle further comprises, and/or is configured to release, a non-vaccine molecule.

65. A method of providing immunity to a virus and/or enhancing an immune response to a virus and/or providing a controlled or sustained release of an antigen in a subject comprising contacting the skin of the subject with the microneedle device of claim 1.

66.-85. (canceled)

86. A method of producing a microneedle device, the method comprising:
providing a mold including a mold body with an array of needle cavities having a predefined shape formed therein;
filling tips of the needle cavities with a composition comprising a coronavirus antigen and/or an influenza antigen;
drying the filled tips of the needle cavities to create microneedle tips, and optionally annealing the microneedle tips;
filling the needle cavities of the mold with a base solution;
drying the base solution to create base layers for the microneedle tips; and
optionally applying a backing to the base layers to create a microneedle device.

87.-93. (canceled)
